(19) United States
(12) Patent Application Publication
(10) Pub. No.: US 2026/0008803 A1
(43) Pub. Date: Jan. 8, 2026

(54) SIALIDASE-RESISTANT SACCHARIDE AND METHOD OF MAKING AND USING THE SAME

(71) Applicant: ACADEMIA SINICA, Taipei (TW)

(72) Inventors: Chi-Huey WONG, Rancho Santa Fe, CA (US); Hong-Jay LO, New Taipei City (TW)

(21) Appl. No.: 19/267,282

(22) Filed: Jul. 11, 2025

Related U.S. Application Data
(62) Division of application No. 17/598,064, filed on Sep. 24, 2021, now Pat. No. 12,384,811.
(60) Provisional application No. 62/829,736, filed on Apr. 5, 2019.

(51) Int. Cl. C07H 15/18; C07H 1/00; C07K 16/28

(52) U.S. Cl. CPC C07H 15/18; C07H 1/00; C07K 16/283; C07K 16/2887; C07K 2317/24; C07K 2317/41; C07K 2317/92

(57) ABSTRACT

Disclosed are a method of preparing a saccharide that contains a 3-fluoro-sialic acid and a method of bonding it to a homogeneous antibody. Also within the scope of this invention are compounds each containing a 3-fluoro-sialic acid, monoclonal antibodies bonded to α2,6-linked 3-fluoro-sialoside terminated N-glycans, and treatment of cancer with such monoclonal antibodies.

### PRIORITY INFORMATION

This application is a divisional of U.S. patent application Ser. No. 17/598,064, filed Sep. 24, 2021, which is a 371 National Phase application of International Application No. PCT/US2020/014608, filed Mar. 10, 2020, claims benefit of and priority to U.S. Provisional Patent Application No. 62/829,736, filed Apr. 5, 2019, the contents of which are incorporated by reference in their entirety.

### BACKGROUND

Sialic acid is a negatively charged monosaccharide often displayed at the outmost end of glycans on glycolipids and glycoproteins, which are involved in many physiological intra- and intercellular processes, including interactions with other biomolecules and receptors on cells, viruses, and bacteria.¹ In addition, sialylation plays an important role in regulating the function and fate of secreted glycoproteins and membrane-bound receptors. For example, sialylation of the epidermal growth factor receptor (EGFR) was shown to inhibit EGFR dimerization, thus interfering with EGF binding and phosphorylation, which is associated with tumorgenesis.² Also, sialylation modulates the half-life of glycoproteins in blood circulation as desialylation of N-glycans exposes the underlining galactose, which is recognized by the hepatic asialoglycoprotein receptors leading to a rapid removal of the glycoprotein from circulation.³ Hence, increasing the degree of sialylation could improve the half-life and undesirable effects of glycoprotein therapeutics.

In recent years, the role of glycosylation on protein structure and function has been intensively studied inspiring the development of new methods for glycan synthesicampillary and glycoengineering of proteins, particularly therapeutic monoclonal antibodies (mAbs).⁵ For instance, it has been shown that afucosylated biantennary N-glycan with terminal α-2,6-linked sialic acids has the optimal glycan structure for the enhancement of antibody-dependent cellular cytotoxicity (ADCC), complement-dependent cytotoxicity (CDC) and anti-inflammatory activities.⁶

Sialic acid derivatives with fluorine at the C-3 position are known to inhibit sialyltransferases and sialidases (or neuraminidases) and are more stable.⁷ However, glycosylation with fluorinated sugars as donors often present a major challenge due to the strong electronic effect of the fluorine group that inactivates glycosylation reaction. Although there is a sialyltransferase capable of transferring a 3-fluoro-sialic acid residue from the corresponding cytidine monophosphate-sialic acid to form an α-2,3-linked sialoside, there is no corresponding α-2,6-sialyltransferase available.¹¹

Thus, there is a need to provide a method for reliably preparing saccharides containing α-2,6-linked 3-fluoro-sialosides, which can be used for developing glycoprotein therapeutics.

### SUMMARY

An aspect of the present invention is a method of preparing a saccharide that contains a 3-fluoro-sialic acid. The method includes conducting a glycosylation reaction by reacting a 3-hydroxy-sialic acid with a saccharide to form an α2,6-linked 3-hydroxy-sialoside and conducting a fluorination reaction by reacting the α2,6-linked 3-hydroxy-sialoside with a fluorinating agent to form a saccharide containing a 3-fluoro-sialic acid.

Another aspect of this invention is a method of preparing a homogeneous antibody bonded to a saccharide that contains a 3-fluoro-sialic acid. The method is carried out by glycosylating a monoclonal antibody with the saccharide.

An additional aspect of this invention relates to compounds of formula (VI):

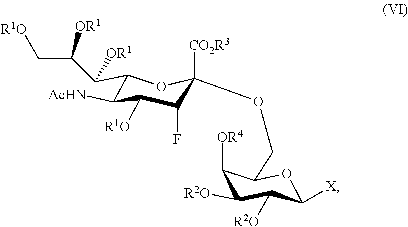

in which R¹ is Ac or H, R² is Bz or H, R³ is methyl or H, R⁴ is Bn or H, and X is OH, a leaving group, or a saccharide. Note that “Ac” represents acetyl, “Bz” represents benzoyl, “Bn” represents benzyl, and the saccharide can be a monosaccharide, a disaccharide, or an oligosaccharide.

Also within the scope of the present invention is a monoclonal antibody bonded to an α2,6-linked 3-fluoro-sialoside terminated N-glycan.

Further covered by this invention is a method of treating a cancer. The method includes administering to a subject in need thereof an effective amount of a monoclonal antibody bonded to an α2,6-linked 3-fluoro-sialoside terminated N-glycan.

The details of the invention are set forth in the description below. Other features, objects, and advantages of the invention will be apparent from the following detailed description of several embodiments, and also from the appending claims.

### BRIEF DESCRIPTION OF THE DRAWINGS

The description below refers to the accompanying drawings, of which:

FIG. 1A shows two graphs depicting enzyme-catalyzed hydrolysis of three sialosides, i.e., 1, 2, and S27, as a function of enzyme concentration; and FIG. 1B shows two graphs depicting sialidase inhibition as a function of the concentration of 2, S27, or 2-deoxy-2,3-didehydro-N-acetylneuraminic acid (DANA), a sialidase inhibitor.

FIG. 2 is a stained electrophoresis gel of four N-glycans: (1) rituxan-G, (2) rituxan-F^{ax}-SCT, (3) rituxan-SCT, and (4) commercial rituxan.

FIG. 3 shows mass spectra obtained for three N-glycans, i.e., rituxan-F^{ax}-SCT, rituxan-SCT, and rituxan.

### DETAILED DESCRIPTION

Disclosed first in detail herein is a method of preparing a saccharide that contains a 3-fluoro-sialic acid.

To reiterate, the method includes the steps of conducting a glycosylation reaction by reacting a 3-hydroxy-sialic acid with a saccharide to form an α2,6-linked 3-hydroxy-sialoside and conducting a fluorination reaction by reacting the α2,6-linked 3-hydroxy-sialoside with a fluorinating agent to form a saccharide containing a 3-fluoro-sialic acid. The saccharide can be a monosaccharide, a disaccharide, or an oligosaccharide. In an exemplary method, the saccharide is a monosaccharide.

In certain embodiments of this method, the 3-hydroxy-sialic acid is a 2-bromo-3-hydroxy-sialic acid, and the glycosylation reaction is conducted in the presence of silver trifluoromethanesulfonate (AgOTf) and disodium phosphate (Na₂HPO₄). Preferably, the glycosylation reaction is conducted in toluene.

In other embodiments of this method, the fluorinating agent is perfluoro-1-butanesulfonyl fluoride (NfF), and the fluorination reaction is conducted in the presence of a catalyst. For example, the catalyst is 1,8-diazabicyclo[5,4,0]-undec-7-ene (DBU). Preferably, the fluorination reaction is conducted in toluene. Of note, this reaction can be conducted further in the presence of tris (dimethylamino) sulfonium difluorotrimethylsilicate (TASF).

In an exemplary method, the 3-hydroxy-sialic acid is of formula (I):

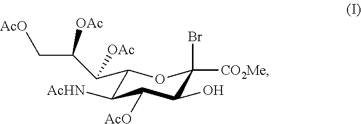

the saccharide is a compound of formula (II):

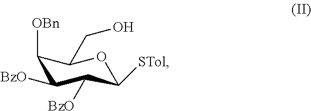

the α2,6-linked 3-hydroxy-sialoside is a compound of formula (III):

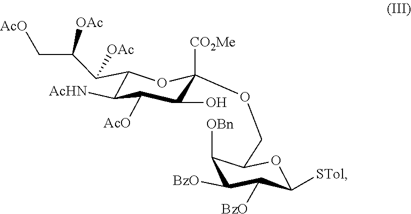

and
the saccharide containing a 3-fluoro-sialic acid is a compound of formula (IV):

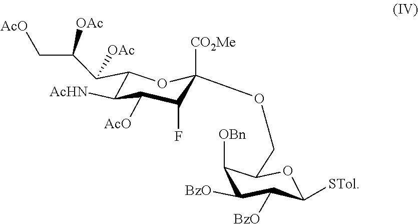

In additional embodiments, the method further includes conducting another glycosylation reaction by reacting the saccharide containing a 3-fluoro-sialic acid with a second saccharide.

Also within the scope of this invention is a method of preparing a homogeneous antibody bonded to a saccharide containing a 3-fluoro-sialic acid. Again, this method includes glycosylating a monoclonal antibody with a saccharide containing a 3-fluoro-sialic acid.

In certain embodiments of this method, the saccharide containing a 3-fluoro-sialic acid is obtained by the method, and embodiments thereof, described above, which includes the steps of conducting a glycosylation reaction by reacting a 3-hydroxy-sialic acid with a saccharide to form an α2,6-linked 3-hydroxy-sialoside and conducting a fluorination reaction by reacting the α2,6-linked 3-hydroxy-sialoside with a fluorinating agent to form a saccharide containing a 3-fluoro-sialic acid.

In other embodiments, the saccharide containing a 3-fluoro-sialic acid is an α2,6-linked 3-fluoro-sialoside terminated N-glycan, which can be of formula (V):

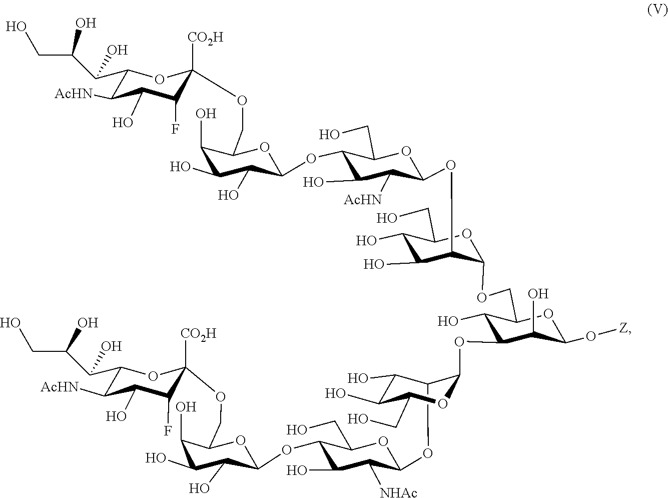

in which Z is

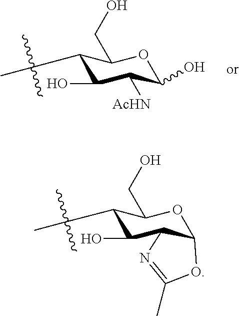

Another aspect of this invention relates to compounds of formula (VI):

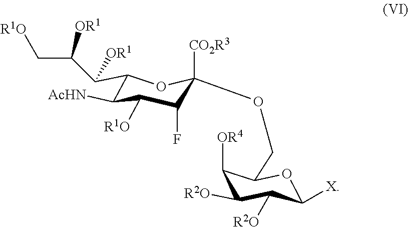

R¹, R², R³, R⁴, and X are defined in the SUMMARY section above.

In one embodiment, the compound is of formula (IV):

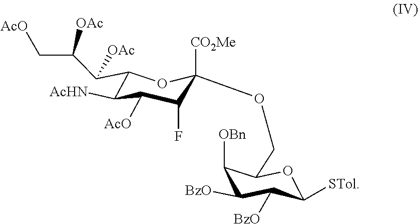

In yet another embodiment, the compounds of formula (VI) each includes X being an N-glycan. An exemplary compound of this embodiment is of formula (V):

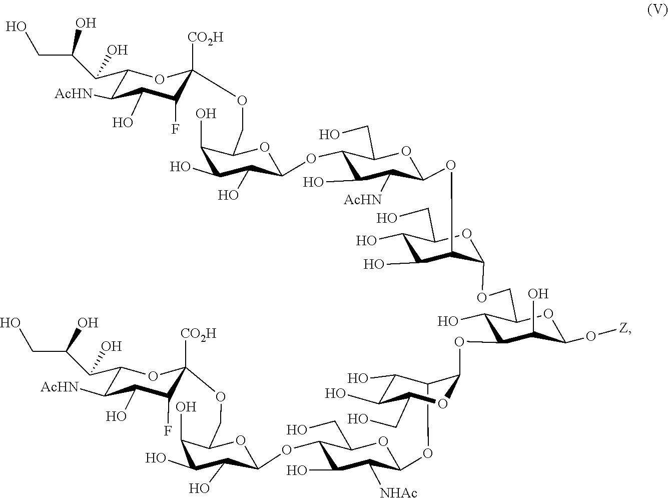

in which Z is

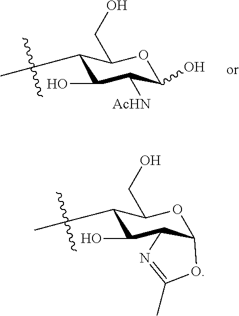

The present invention also covers a monoclonal antibody bonded to a saccharide containing a 3-fluoro-sialic acid, the saccharide containing a 3-fluoro-sialic acid being an α2,6-linked 3-fluoro-sialoside terminated N-glycan. Preferably, the α2,6-linked 3-fluoro-sialoside terminated N-glycan is of formula (V) shown above.

Further covered by this invention is a method of treating a cancer or an autoimmune disease, which includes administering to a subject in need thereof an effective amount of an above-described monoclonal antibody. For example, the cancer can be leukemia or lymphoma, and the autoimmune disease can be rheumatoid arthritis, autoimmune hemolytic anemia, pure red cell aplasia, thrombotic thrombocytopeniarpura, idiopathic thrombocytopeniarpura leukemia, lymphoma, Evans syndrome, vasculitis, bullous skin disorders, type 1 diabetes mellitus, Sjogren's syndrome, anti-NMDA receptor encephalitis, Devic's disease, Graves' ophthalmopathy, autoimmune pancreatitis, opsoclonus myoclonus syndrome, or a IgG4-related disease.

The term “treating” or “treatment” herein refers to administering a pharmaceutical composition described above to a subject, who has an above-described disease, i.e., cancer, a symptom of such a disease, or a predisposition toward such a disease, with the purpose of conferring a therapeutic or prophylactic effect. The term “an effective amount” refers to the amount of an active drug that is required to confer such effect. Effective doses will vary, as recognized by those skilled in the art, depending on the types of disease treated, route of administration, excipient usage, and the possibility of co-usage with other therapeutic treatment.

Provided below are more detailed descriptions of certain specific aspects of the present invention.

3-fluoro-substitued sialic acid derivatives, compared to non-fluorinated sialic acid derivatives, are known to be more stable towards sialidases.⁷ By introducing the fluorine atoms to the anomeric and C-3 positions, a 2,3-difluorosialic acid (DFSA) was developed (Scheme 1)⁸ and used as a biochemical probe,⁹ and the activity-based protein profiling probe to study sialidases.¹⁰ With the help of DFSA, it was shown that the fluorine atom at the axial position at C-3 (3F^{ax}) has a greater effect on slowing down both the deactivation (kᵢ) and reactivation (k_{hydr}) of the enzyme than the 3F^{eq} derivative.^{8b} Inspired by this observation, we wanted to investigate the stability of sialosides with 3-fluorosialic acids for their potential use in glycoprotein therapeutics. Specifically, we intend to study if incorporation of a 3F^{ax}Neu5Ac motif at the terminal end of N-glycan on a mAb could increase its stability towards sialidases and sustain its effector functions.

Towards our goal, we developed a preparative method for the synthesis of α2,6-linked 3F^{ax}-Neu5Ac oligosaccharides, including the biantennary N-glycan. We also showed that the synthetic 3-F^{ax}Neu5Ac-α2,6-Gal linkage is more stable in the presence of sialidases and the antibody with the corresponding biantennary glycan has the same binding avidity as that of the non-fluorinated counterpart.

Several methods were reported for the synthesis of 3F-Neu5Ac, including (a) fluorination of protected glycans with XeF₂—BF₃×OEt₂,¹² molecular fluorine¹³ and Selectfluor;^{7c} (b) inversion of equatorial hydroxyl group at C3 in a sialic acid derivative;^{7f} and (c) aldolase-catalyzed enzymatic transformation of ManNAc and 3-fluoro-pyruvate into 3F^{eq}Neu5Ac and 3FaxNeu5Ac.^{7a, 11a, 14} However, to the best of our knowledge, there is no method describing the synthesis of N-glycans terminated with 3F^{ax}-Neu5Ac so far. The only account disclosing the preparation of disaccharides with 3F^{ax}-Neu5Ac was limited to the enzymatic synthesis of 3-FaxNeu5Ac-α2,3-Lac, and not the required α2,6-linkage.^{11a} We, therefore, focused our effort on the chemical synthesis of this linkage.¹⁵

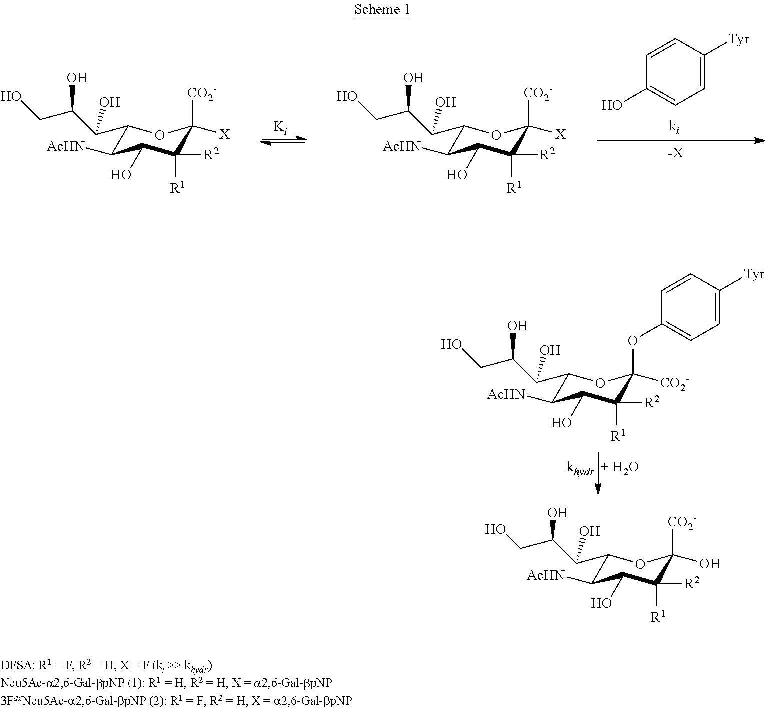

After screening a variety of glycosylation conditions using 3F^{ax}-Neu5Ac-based donors (Scheme 2a), we investigated the alternative strategies, which encompassed the S_{N}2 reaction of the 3OH^{eq} to 3F^{ax} in 3OH-Neu5Ac-α2,6-Gal-STol (Scheme 2b). First, we studied the sialylation at O-6 using conditions reported by Goto et al. (Table 1, entry 1).¹⁶ The reaction was carried out using 1 equiv. of AgOTf as a promoter and Na₂HPO₄ as a base in toluene at −10° C. (Scheme 3). These conditions provided disaccharide 6 in low yield (15%) with a 3:1 (α:β) anomeric ratio. Changing the solvent to CH₂Cl₂ did not improve the diastereoselectivity (entry 2). By increasing the temperature and reaction time, the yield was improved, however, the selectivity decreased dramatically (entries 3-5). Surprisingly, the more equivalents of acceptor were used, the better stereoselectivity was observed (entry 3 vs. 6). The optimized conditions (entry 7) provided 6 in 35% (99% brsm) yield with excellent α-selectivity (α:β=13:1).

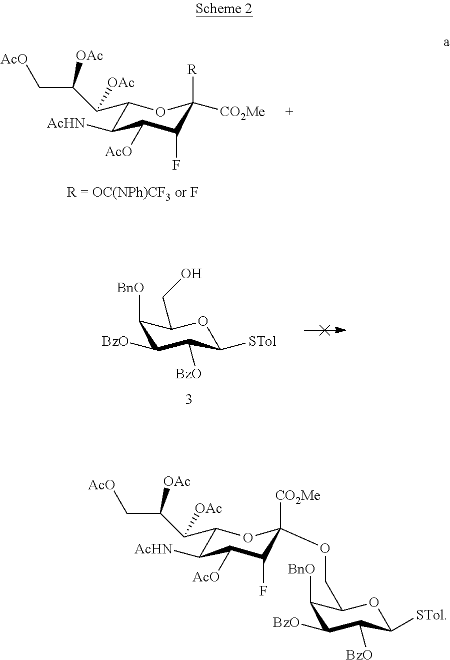

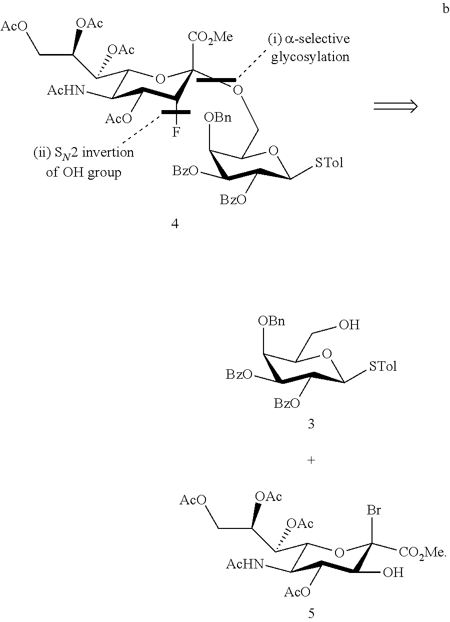

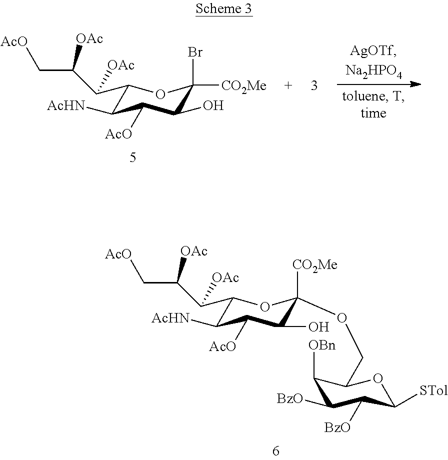

TABLE 1
Optimization of the glycosylation step.

| | 5/3/AgOTf/ | | | | Yield^{b} |
|---|---|---|---|---|---|
| | Na₂HPO₄, | T | time | | (%) |
| entry | (equiv.) | ( ° C.) | (h) | α:β^{a} | (brsm)^{c} |
| 1 | 1/1.1/1/4.6 | −10 | 0.5 | 3.1:1 | 15^{d} |
| 2^{e} | 1.1/1/1.1/— | −78 to −20 | 3 | 2.7:1 | 21^{d} |
| 3 | 0.8/1/1.6/4.5 | −50 | 16 | 5.2:1 | 46 (94) |
| 4 | 0.7/1/1.5/4.2 | −50 to −35 | 16 | 3.8:1 | 53 (99) |
| 5 | 0.7/1/1.5/4.2 | −50 to −15 | 16 | 1.8:1 | 72 (99) |
| 6 | 1/1.25/1.5/4.2 | −50 | 16 | 16:1 | 28 |
| 7 | 1/1/1.5/4.2 | −50 | 16 | 13:1 | 35 (99) |
| 8^{f} | 1/1/1.5/— | −50 | 16 | 11:1 | 33 (99) |
| 9^{g} | 1/1/1.5/4.2 | −50 | 24 | 13:1 | 35 (99) |

^{a}Determined by ¹H NMR.
^{b}Yield of isolated product.
^{c}Yield based on the recovered starting material (brsm), acceptor.
^{d}The donor and acceptor were not completely consumed.
^{e}Using CH₂Cl₂ with 4 Å MS.
^{f}With 4 Å MS.
^{g}Gram-scale.

Inversion of OH^{eq} to F^{ax} turned out to be a challenging task. Substitution of OTf and OMs by fluorine using TASF led to decomposition of the starting material. To optimize this transformation, we screened a variety of fluorinating reagents without any success. However, treatment of 6 with perfluoro-1-butanesulfonyl fluoride (NfF) in the presence of 1,8-diazabicyclo[5,4,0]-undec-7-ene (DBU) in anhydrous toluene (Scheme 3) for 2 days at 90° C. gave 4 in 6% yield (Table 2, entry 1).¹⁷ Further optimization of the reaction conditions, such as decreasing reaction temperature (entries 1, 5 and 6) and increasing reaction times (entry 7), improved the overall yield of 4. It seems that the transformation of 7 to 4 is a rate-limiting step due to the steric hindrance around C-3. In many cases, we were able to isolate 7 formed in the presence of NfF and DBU at room temperature within 1 day. However, conversion of 7 to 4 (77% brsm yield) requires long reaction times. The attempts to improve the conversion by elevating reaction temperature resulted in decomposition of 7. Thus, our best result was obtained by increasing amounts of reagents and reaction times to 15 days (entry 8). Finally, we found that addition of TASF helped improve the reaction efficiency reducing the reaction time to only 2 days (entry 9). The stereochemistry of fully protected 3F^{ax}-Neu5Ac-α2,6-Gal-STol disaccharide (4) was verified by the X-ray diffraction analysis.

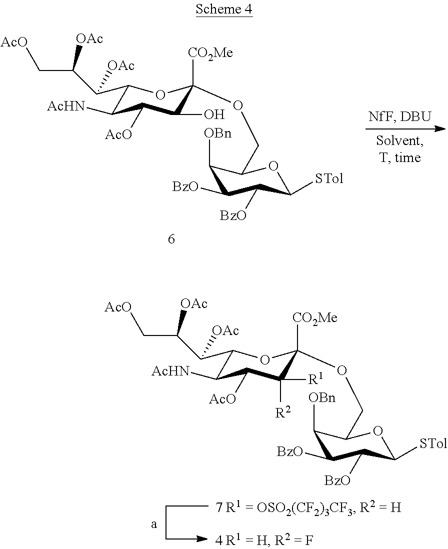

TABLE 2
Optimization of the fluorination step.

| | NfF/DBU, | | T | time | Yield,^{b} |
|---|---|---|---|---|---|
| entry | (equiv.) | solvent | (° C.) | (day) | (%) 4:7 |
| 1 | 6/6 | THF | 25 to 90 | 2 | 4 |
| 2 | 6/6 | CH₃CN | 25 to 90 | 2 | 2 |
| 3 | 6/6 | toluene | 25 to 90 | 2 | 6 |
| 4 | 4/4 | DMF | 40 | 9 | —^{c} |
| 5 | 4/4 | toluene | 50 | 6 | 8:71 |
| 6 | 4/4 | toluene | 40 | 7 | 30:52 |
| 7 | 4/4 | toluene | 40 | 17 | 36:44 |
| 8^{d} | 8/8 | toluene | 40 | 15 | 63:15 |
| 9^{e} | 8/8 | toluene | 40 | 2 | 60:8 |

^{a}Reagents and conditions: (a) NfF, DBU, toluene, 40° C., 15 d, 49% (77% brsm).
^{b}Yield of isolated product.
cNo product observed.
^{d}Portion-wise addition of NfF (4 equiv./day) and DBU (4 equiv./day) followed by stirring for 14 days.
^{e}Portion-wise addition of NfF (4 equiv./day), DBU (4 equiv./day) and tris(dimethylamino)sulfonium difluorotrimethylsilicate (TASF) (2 equiv./day).

Referring to Scheme 5, the 3F^{ax}-Neu5Ac-disaccharide donor 4 was coupled with the acceptor (8) using NIS/TMSOTf to give 9. Next, the O-allyl group at the anomeric position was removed by isomerization with PdCl₂ in AcOH/NaOAc, and the anomeric hydroxyl group (10) was further transformed into fluoride (11) and imidate (12). The glycosylation of the core disaccharide (13) at O-3 with 3F^{ax}-Neu5Ac-terminated fluoride donor (11) using Cp₂HfCl₂/AgOTf conditions gave hexasaccharide 14 in 85% yield. After removal of the benzylidene group, 15 was glycosylated at the O-6 position to give the desired decasaccharide (16) in 70% yield with excellent regio- and α-stereoselectivity. We also tested the TfOH-promoted glycosylation with N-phenyl trifluoroacetimidate donor (12), which, however, gave the product in a poor yield. Next, the fully deprotected glycan (17) was obtained in 40% overall yield following a sequence of steps: (a) saponification with LiOH to remove the esters and the NHTroc group; (b) acetylation of free amines and alcohols; (c) removal of the OAc groups with sodium methoxide; and (d) hydrogenolysis of the O-benzyl groups with Pd/C in a mixture of MeOH/water/HCO₂H.^{4b}

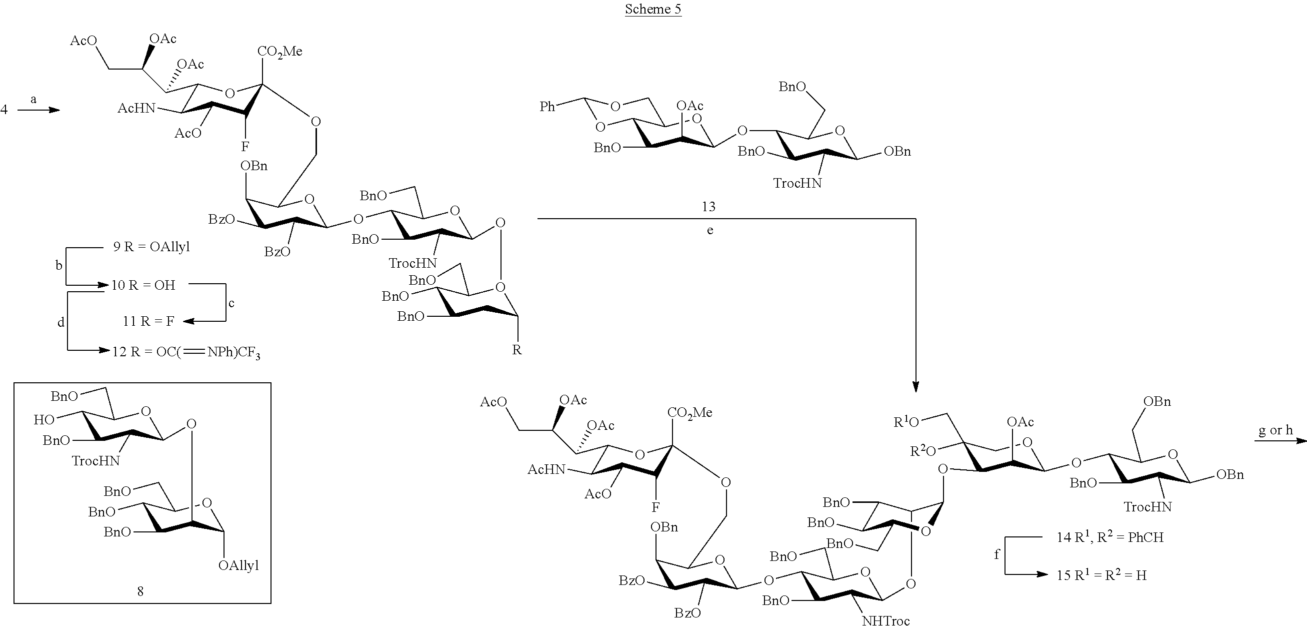

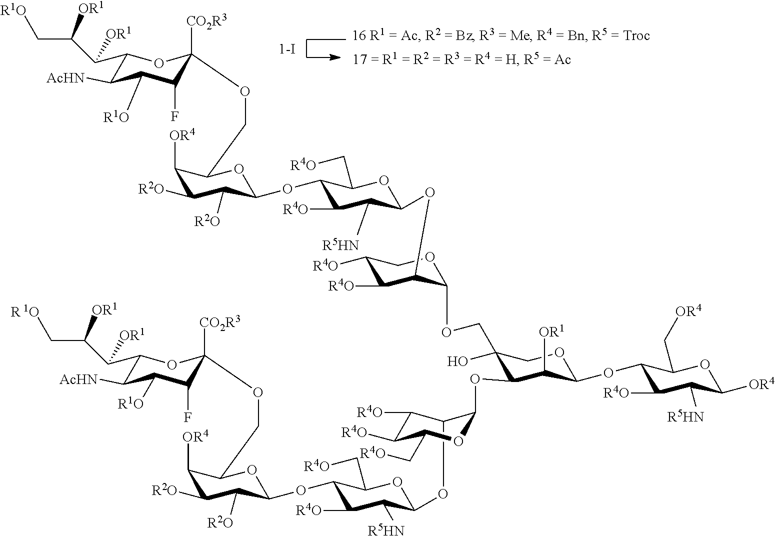

Reagents and conditions in Scheme 5: (a) 8, TfOH, NIS, 4 Å MS, CH₂Cl₂, −40° C., 2 h, 64%. (b) PdCl₂, CH₃COONa, AcOH/H₂O, 82%. (c) DAST, CH₂Cl₂, −20° C., 73%. (d) ClC(═NPh)CF₃, Cs₂CO₃, CH₂Cl₂, 0° C. to r.t., 3 h, 56%; (e) 11, AgOTf, Cp₂HfCl₂, toluene, 4 Å MS, 0° C., 3 h, 85%; (f) pTSA·H₂O, CH₃CN, r.t., 6 h, 75%; (g) 11, AgOTf, Cp₂HfCl₂, toluene, 4 Å MS, −15° C., 3 h, 70% (80% brsm); (h) 12, TfOH, CH₂Cl₂, 4 Å MS, −60 to −20° C., 3 h, 33% (55% brsm); (i) LiOH, dioxane/H₂O (4:1), 90° C., 16 hrs; (j) Ac2O, Py, 16 h; (k) NaOMe, MeOH, 16 h; (1) Pd(OH)₂, MeOH/H₂O/HCOOH (6:3:1), H₂, 16 h, 40% (4 steps).

Having established a protocol for the stepwise synthesis, we streamlined the glycan assembly by developing a programmable [2+2+2] one-pot synthesis of hexasaccharide (14), which is a precursor of 17 (Scheme 6). The designed one-pot protocol was comprised of the initial coupling of the 3F^{ax}-Neu5Ac-disaccharide donor (4) (RRV=2053) with a less reactive acceptor (18) (RRV=537) at −40° C., followed by injection of the reducing-end acceptor 13 at −20° C. After 1 h at −10° C. and a standard purification protocol, the hexasaccharide 14 was isolated in 26% yield.

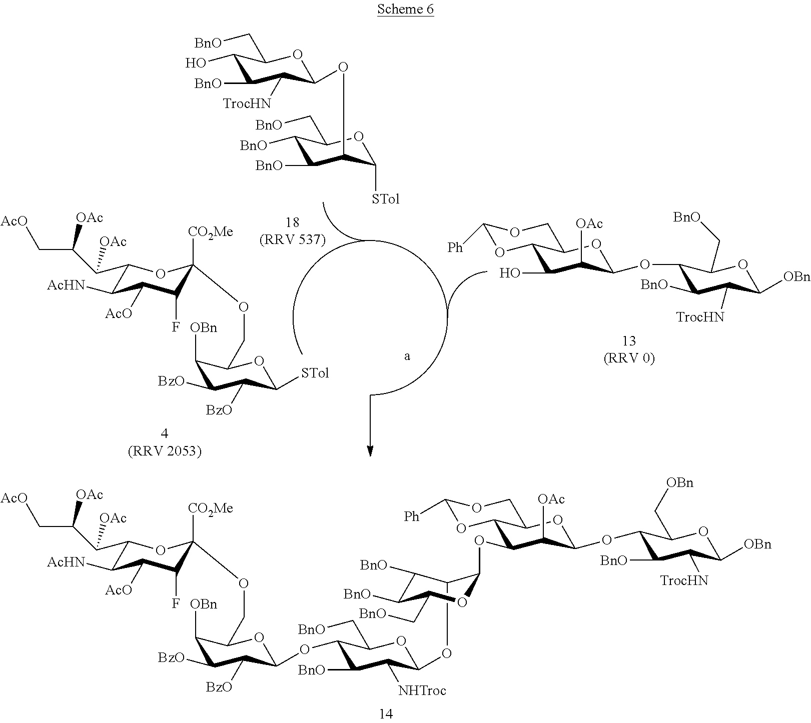

Reagents and conditions in Scheme 6: (a) TfOH, NIS, 4 Å MS, CH₂Cl₂, −40° C. to −10° C., 3 h, 26%

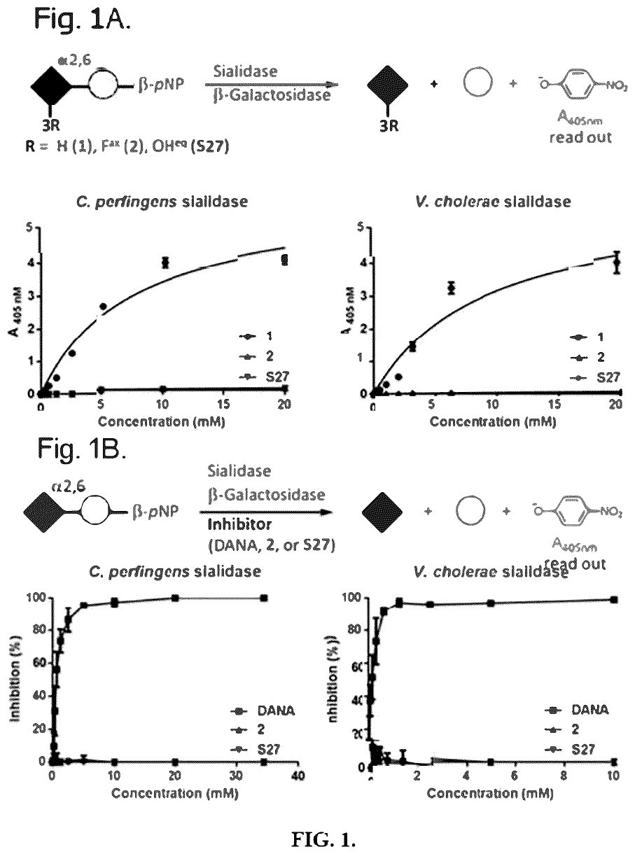

In order to gather preliminary data about the stability of the 3F^{ax}-Neu5Ac-α2,6-Gal motif in the presence of sialidases, we prepared Neu5Ac-α2,6-Gal-pNP (1) and the 3F^{ax}-Neu5Ac analog (2) as substrates (Scheme 1) for the in vitro assay¹⁹ with the commercially available sialidases from C. perfingens and V. cholera. Both enzymes showed the expected hydrolytic activity for the native substrate 1 but were inactive toward the 3F^{ax}-analog 2 (see FIG. 1A). We also observed that 2 did not significantly inhibit the hydrolysis of 1 as DANA did (see FIG. 1B).

To prepare a homogeneous glycoform of mAb, compound 17 was converted into the oxazoline donor and ligated to the GlcNAc-primed IgG (without core fucose) in the presence of Endo S2 (D184Q) following a standard protocol.⁶ The binding avidity of the homogeneous 3F^{ax}-Neu5Ac-glycoform to FcγRIIIa was measured by surface plasma resonance analysis⁶ together with the parent nonfluorinated glycoform (G2S2) and a commercial sample of rituximab (major glycoforms: G1F1, G0F1, G2F1). When compared to the commercial sample of rituximab, the homogeneous glycoforms of IgG bearing biantennary N-glycans with α2,6-sialaylation and without core fucose demonstrated 39.9-fold (Neu5Ac-G2S2) and 37.4-fold (3F^{ax}-Neu5Ac-G2S2) improvement in binding avidity (see Table 4). The fact that the avidity of the 3F^{ax}-Neu5Ac-modified glycoform was found to be within the same range as that of the parent glycan provides a premise for the in vivo studies of 3F^{ax}-Neu5Ac-glycosylated mAb. These results will be reported in a due course.

In conclusion, we have developed a chemical synthesis of 3F^{ax}-Neu5Ac-α2,6-Gal-STol as building block for the synthesis of sialidase-resistant oligosaccharides and a representative homogeneous antibody with 3F^{ax}-Neu5Ac-terminated bi-antennary N-glycan. When compared with the commercial rituximab sample, the homogeneous glycoform modified with 3F^{ax}-Neu5Ac-glycans showed a 37.4-fold improvement in binding to the FcγRIIIa. Moreover, the parent nonfluorinated and 3F^{ax}-Neu5Ac-modified antibody glycoforms demonstrated similar binding avidity to an Fc receptor. Overall, our results have revealed a new general strategy for the improvement of half-lives of therapeutic glycoproteins.

Without further elaboration, it is believed that one skilled in the art can, based on the above description, utilize the present invention to its fullest extent. The following specific examples are, therefore, to be construed as merely illustrative, and not limitative of the remainder of the disclosure in any way whatsoever. All publications cited herein are incorporated by reference in their entirety.

### EXAMPLE 1

### Synthesis of Saccharides Containing a 3-fluoro-sialic acid and Precursors

All reactions were carried out under an inert atmosphere unless mentioned otherwise, and standard syringe-septa techniques were followed. Solvents were purchased from commercial sources and used without further purification. Pulverized molecular sieves 4 Å (EMD Millipore) were grounded in powder and activated before use. The progress of all the reactions was monitored by TLC glass plates precoated with silica gel 60 F254 (Merck KGaA). The TLC was visualized by UV light (254 nm), p-anisaldehyde and/or ceric ammonium molybdate stains. Column chromatography was performed on Across silica gel (particle size 0.035-0.070 mm, 60 Å). ¹H, ¹³C, and ¹⁹F NMR spectra were recorded with Bruker AVIII-600, DRX-500, AV-400, DPX-400, AVANCE 500 AV and AVANCE 600 spectrometers at 25° C. and chemical shifts were measured in 8 (ppm) with residual solvent peaks as internal standards (δ, ppm: 7.24 (CHCl₃), 4.80 (H₂O) in ¹H NMR; and 77 (CDCl₃) in ¹³C NMR). Coupling constants (J) were measured in Hz. Data are represented as follows: chemical shift, multiplicity (s=singlet, d=doublet, t=triplet, q=quartet, m=multiplet, br=broad). High-resolution mass spectra (HRMS) were recorded on an Agilent LC/MSD TOF mass spectrometer by electrospray ionization time-of-flight (ESI-TOF) reflectron experiments. The single crystal X-ray diffraction studies were carried out on a Bruker D8 Platinum-¹³⁵ CCD diffractometer equipped with Cu Kα radiation (□=1.5478). HPLC measurements were performed on a Hitachi HPLC D-7000 system. RRV measurements were recorded using a normal-phase ZORBAX RX-SIL, 5 μm, 4.6×250 mm (Colloidal Silica, Agilent Technologies) using the solvent system EtOAc/hexane with 1 mL/min flow rate, and visualized at 254 nm.

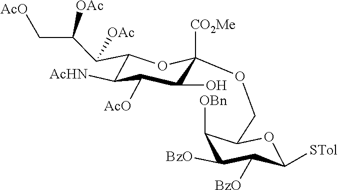

p-Tolyl [methyl 5-acetamido-4,7,8,9-tetra-O-acetyl-5-deoxy-D-erythro-α-L-gluco-non-2-ulopyranosonate]-(2→6)-2,3-di-O-benzoyl-4-O-benzyl-1-thio-β-D-galactopyranoside 6α. A mixture of acceptor 3 (1.05 g, 1.80 mmol, 1 equiv.) and donor 5 (1.02 mg, 1.80 mmol, 1 equiv.) in anhydrous toluene (60 mL) was stirred under argon atmosphere for 10 min. Then, the reaction mixture was cooled to −50° C. and dry Na₂HPO₄ (1.07 g, 7.54 mmol, 4.2 equiv.) was added, followed by AgOTf (692 mg, 2.69 mmol, 1.5 equiv., in toluene 18 mL) with stirring. Upon completion of the reaction (TLC indicated the disappearance of starting materials after 24 hrs), the reaction mixture was diluted with EtOAc (80 mL), washed with 20% aq. Na₂S₂O₃ (10 mL), satd. aq. NaHCO₃ (5 mL) and brine (5 mL), then the organic layer was dried over MgSO₄, filtered, and concentrated. The obtained residue was purified by silica gel column chromatography using acetone/toluene (3:7) as eluent to give compound 6 (α:β=13:1) as a white powder (680 mg, 35%) along with recovered acceptor 3 (670 mg, 99% brsm). α-anomer 6α: R_{f}=0.36 (silica gel, CHCl₃:MeOH=20:1); ¹H NMR (600 MHz, CDCl₃): δ 7.96-7.94 (m, 2H, Ar—H), 7.91-7.90 (m, 2H, Ar—H), 7.50-7.47 (m, 2H, Ar—H), 7.40-7.39 (m, 2H, Ar—H), 7.37-7.32 (m, 5H, Ar—H), 7.25-7.18 (m, 4H, Ar—H), 7.06-7.04 (m, 2H, Ar—H), 5.81 (dd, J=10.2. 9.6 Hz, 1H), 5.59 (d, J=10.2 Hz, 1H), 5.38 (dd, J=9.0, 3.0 Hz, 1H), 5.35-5.32 (m, 1H), 5.25 (dd, J=10.2, 9.6 Hz, 1H), 5.24 (d, J=7.8, 2.4 Hz, 1H), 4.92 (d, J=9.6 Hz, 1H, C1-H_{β}), 4.71 (d, J=12.0 Hz, 1H), 4.60 (d, J=12.0 Hz, 1H), 4.49 (dd, J=10.8, 1.8 Hz, 1H), 4.30 (dd, J=12.6, 3.0 Hz, 1H), 4.26 (ddd, J=10.2, 10.2, 10.2 Hz, 1H), 4.20 (d, J=3.0 Hz, 1H), 4.10 (dd, J=10.2, 7.2 Hz, 1H), 4.04 (dd, J=12.6, 6.6 Hz, 1H), 4.00 (dd, J=6.6, 6.6 Hz, 1H), 3.86 (d, J=9.6 Hz, 1H), 3.82-3.73 (m, 2H), 3.75 (s, 3H, —CH₃), 2.30 (s, 3H, —CH₃), 2.09 (s, 3H, —CH₃), 2.07 (s, 3H, —CH₃), 2.06 (s, 3H, —CH₃), 1.96 (s, 3H, —CH₃), 1.89 (s, 3H, —CH₃); ¹³C NMR (150 MHz, CDCl₃): δ 171.6, 170.6, 170.3, 170.1, 169.5, 168.5, 165.8, 165.1, 138.0, 137.9, 133.4, 133.1, 129.8, 129.7, 129.6, 128.9, 128.6, 128.4, 128.3, 128.2, 127.7, 127.6, 100.3, 86.6, 75.9, 74.5, 73.7, 73.2, 73.1, 72.5, 68.8, 68.3, 63.1, 62.5, 52.8, 48.4, 23.1, 21.2, 20.9, 20.8, 20.7, 20.7; HRMS (ESI-TOF) m/e: Calcd for C₅₄H₅₉NO₂₀SNa [M+Na]⁺: 1096.3243 found 1096.3241.

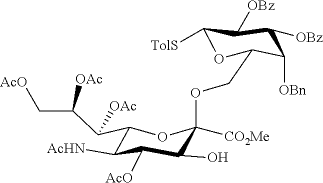

p-Tolyl [methyl 5-acetamido-4,7,8,9-tetra-O-acetyl-5-deoxy-D-erythro-β-L-gluco-non-2-ulopyranosonate]-(2→6)-2,3-di-O-benzoyl-4-O-benzyl-1-thio-β-D-galactopyranoside 6β. β-anomer 6β R_{f}=0.42 (silica gel, CHCl₃:MeOH=20:1); ¹H NMR (600 MHz, CDCl₃): δ 7.94-7.92 (m, 4H, Ar—H), 7.50-7.47 (m, 2H, Ar—H), 7.43-7.40 (m, 6H, Ar—H), 7.37-7.31 (m, 5H, Ar—H), 7.13-7.12 (m, 2H, Ar—H), 5.81 (dd, J=10.2. 10.2 Hz, 1H), 5.51 (dd, J=10.2, 3.0 Hz, 1H), 5.22 (ddd, J=6.0, 6.0, 3.0 Hz, 1H), 5.14 (d, J=13.2 Hz, 1H), 5.07 (dd, J=6.0, 2.4 Hz, 1H), 4.90 (d, J=10.2 Hz, 1H, C1-H_{β}), 4.66-4.62 (m, 2H), 4.55 (dd, J=12.6, 2.4 Hz, 1H), 4.29 (d, J=1.8 Hz, 1H), 4.14 (dd, J=7.8, 7.2 Hz, 1H), 4.08 (ddd, J=10.8, 10.8, 10.8 Hz, 1H), 3.98 (dd, J=12.6, 6.6 Hz, 1H), 3.92-3.88 (m, 2H), 3.85-3.77 (m, 3H), 3.83 (s, 3H, —CH₃), 3.53 (dd, J=10.8, 2.4 Hz, 1H), 2.32 (s, 3H, —CH₃), 2.26 (s, 3H, —CH₃), 2.07 (s, 3H, —CH₃), 2.04 (s, 3H, —CH₃), 1.97 (s, 3H, —CH₃), 1.68 (s, 3H, —CH₃); ¹³C NMR (150 MHz, CDCl₃): δ 171.4, 170.6, 170.3, 169.8, 169.8, 166.6, 165.8, 165.2, 139.2, 138.4, 133.5, 133.4, 133.1, 129.9, 129.8, 129.5, 128.9, 128.5, 128.3, 127.2, 126.0, 99.6, 86.9, 76.0, 75.5, 74.3, 73.1, 72.5, 71.7, 70.3, 68.4, 67.2, 62.1, 62.0, 53.3, 47.5, 22.9, 21.5, 21.2, 20.8, 20.7, 20.7; HRMS (ESI-TOF) m/e: Calcd for C₅₄H₅₉NO₂₀S [M+H]⁺: 1074.3424 found 1074.3458.

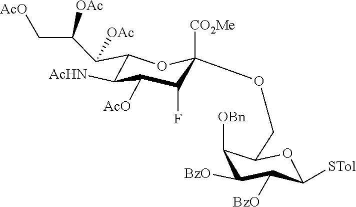

p-Tolyl [methyl 5-acetamido-4,7,8,9-tetra-O-acetyl-3,5-dideoxy-3-fluoro-D-erythro-α-L-manno-non-2-ulopyranosonate]-(2→6)-2,3-di-O-benzoyl-4-O-benzyl-1-thio-β-D-galactopyranoside 4. To a solution of compound 6α (500 mg, 0.47 mmol, 1 equiv.) in toluene (5 mL) in a screw-capped vial containing a stirring bar was added DBU (0.28 mL, 1.86 mmol, 4 equiv.), perfluoro-1-butanesulfonyl fluoride (0.33 mL, 1.86 mmol, 4 equiv.) and TASF (256 mg, 0.93 mmol, 2 equiv.). The reaction vial was sealed and stirred at 40° C. After 24 h, the reaction mixture was treated with additional amounts of DBU (0.28 mL, 1.86 mmol, 4 equiv.), perfluoro-1-butanesulfonyl fluoride (0.33 mL, 1.86 mmol, 4 equiv.) and TASF (256 mg, 0.93 mmol, 2 equiv.), then stirred at 40° C. for another 24 h. The reaction mixture was directly loaded onto the silica gel column and eluted with acetone/toluene (7:3). The disaccharide 4 was obtained as a light yellow powder (300 mg, 60%) along with perfluoro-1-butanesulfonyl compound 7 as a light yellow powder (50 mg, 8%).

Synthesis of the disaccharide 4 from 7: To a solution of compound 7 (730 mg, 0.54 mmol, 1 equiv.) in toluene (7 mL) in a screw-capped vial containing a stirring bar was added DBU (0.64 mL, 4.31 mmol, 8 equiv.) and perfluoro-1-butanesulfonyl fluoride (0.77 mL, 4.31 mmol, 8 equiv.). The container was sealed and stirred at 40° C. for 15 days. The reaction mixture was directly loaded onto the silica gel column and eluted with acetone/toluene (7:3) as eluent; disaccharide 4 was isolated as a light yellow powder (282 mg, 49%) along with perfluoro-1-butanesulfonyl compound 7 as a light yellow powder (267 mg, 77% brsm). R_{f}=0.43 (silica gel, acetone/toluene=2:3); ¹H NMR (600 MHz, CDCl₃): δ 7.95-7.93 (m, 2H, Ar—H), 7.87-7.85 (m, 2H, Ar—H), 7.48-7.44 (m, 2H, Ar—H), 7.41-7.39 (m, 2H, Ar—H), 7.35-7.33 (m, 2H, Ar—H), 7.31-7.29 (m, 2H, Ar—H), 7.25-7.24 (m, 2H, Ar—H), 7.21-7.19 (m, 2H, Ar—H), 7.16-7.14 (m, 1H, Ar—H), 7.04-7.03 (m, 2H, Ar—H), 5.79 (dd, J=10.2. 9.6 Hz, 1H), 5.49 (ddd, J=9.0, 5.4, 2.4 Hz, 1H), 5.42 (dd, J=10.2, 3.0 Hz, 1H), 5.31-5.28 (m, 2H), 5.20 (dd, J=27.0, 11.4 Hz, 1H, sia-C4-H), 5.01 (dd, J=51.6, 1.8 Hz, 1H, sia-C3-H), 4.97 (d, J=10.2 Hz, 1H, C1-H_{β}), 4.66 (d, J=11.4 Hz, 1H), 4.58 (d, J=11.4 Hz, 1H), 4.37 (dd, J=12.6, 2.4 Hz, 1H), 4.28-4.26 (m, 2H), 4.17 (dd, J=12.6, 5.4 Hz, 1H), 4.09-4.05 (m, 2H), 3.98 (dd, J=10.2, 6.0 Hz, 1H), 3.74 (dd, J=10.2, 8.4 Hz, 1H), 3.72 (s, 3H, —CH₃), 2.29 (s, 3H, —CH₃), 2.17 (s, 3H, —CH₃), 2.16 (s, 3H, —CH₃), 2.09 (s, 3H, —CH₃), 1.98 (s, 3H, —CH₃), 1.91 (s, 3H, —CH₃); ¹³C NMR (150 MHz, CDCl₃): δ 170.9, 170.7, 170.4, 170.2, 169.8, 165.6, 165.3, 138.3, 137.6, 133.2, 133.0, 132.6, 129.8, 129.8, 129.7, 129.5, 129.3, 129.1, 128.4, 128.3, 128.1, 127.5, 127.3, 98.3, 98.2, 88.1, 86.8, 86.4, 76.2, 75.6, 74.6, 74.0, 71.4, 69.2, 69.0, 68.5, 68.0, 67.3, 63.4, 62.5, 53.2, 45.5, 23.4, 21.2, 21.2, 20.8, 20.7, 20.7; ¹⁹F NMR (376 MHz, CDCl₃): ϵ −215.9; HRMS (ESI-TOF) m/e: Calcd for C₅₄H₅₈FNO₁₉SNa [M+Na]⁺: 1098.3200 found 1098.3212.

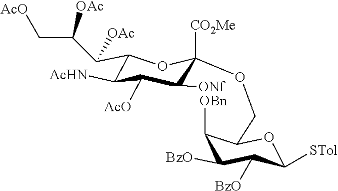

p-Tolyl [methyl 5-acetamido-4,7,8,9-tetra-O-acetyl-5-deoxy-3-O-(perfluoro-1-butane) sulfonyl-D-erythro-α-L-gluco-non-2-ulopyranosonate]-(2→6)-2,3-di-O-benzoyl-4-O-benzyl-1-thio-β-D-galactopyranoside 7. R_{f}=0.59 (silica gel, acetone:toluene=2:3); ¹H NMR (600 MHz, CDCl₃): δ 7.95-7.94 (m, 2H, Ar—H), 7.90-7.89 (m, 2H, Ar—H), 7.49-7.45 (m, 2H, Ar—H), 7.40-7.39 (m, 2H, Ar—H), 7.36-7.30 (m, 4H, Ar—H), 7.27-7.26 (m, 2H, Ar—H), 7.25-7.18 (m, 3H, Ar—H). 7.04-7.03 (m, 2H, Ar—H), 5.82 (dd, J=10.2. 9.6 Hz, 1H), 5.62-5.38 (m, 4H), 5.26 (d, J=9.0 Hz, 1H), 4.96-4.94 (m, 2H, C1-H_{β}), 4.70-4.64 (m, 3H), 4.35-4.30 (m, 1H), 4.22-4.20 (m, 2H), 4.09 (dd, J=9.6, 6.0 Hz, 1H), 4.02 (dd, J=12.6, 6.0 Hz, 1H), 3.98 (dd, J=6.6, 6.0 Hz, 1H), 3.93 (dd, J=10.2, 7.2 Hz, 1H), 3.76 (s, 3H, —CH₃), 2.29 (s, 3H, —CH₃), 2.17 (s, 3H, —CH₃), 2.09 (s, 3H, —CH₃), 2.06 (s, 3H, —CH₃), 1.94 (s, 3H, —CH₃), 1.90 (s, 3H, —CH₃); ¹³C NMR (150 MHz, CDCl₃): δ 170.6, 170.2, 170.1, 169.3, 167.0, 165.7, 165.2, 138.1, 137.7, 133.2, 133.0, 132.5, 129.8, 129.7, 129.7, 129.5, 129.1, 129.0, 128.4, 128.3, 128.1, 128.0, 127.5, 127.4, 97.8, 86.5, 83.4, 75.7, 74.6, 74.2, 72.6, 70.0, 68.4, 67.9, 66.5, 64.5, 62.4, 53.1, 49.0, 23.0, 21.1, 20.9., 20.7, 20.6, 20.3; ¹⁹F NMR (376 MHz, CDCl₃): δ −126.2, −126.2, −121.5, −121.5, −110.4, −110.4, −81.0, −80.9, −80.9; HRMS (ESI-TOF) m/e: Calcd for C₅₈H₅₈F₉NO₂₂S₂Na [M+Na]⁺: 1378.2640 found 1378.2644.

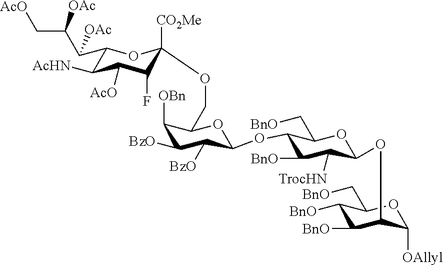

Allyl [methyl 5-acetamido-4,7,8,9-tetra-O-acetyl-3,5-dideoxy-3-fluoro-D-erythro-α-L-manno-non-2-ulopyranosonate]-(2→6)-[2,3-di-O-benzoyl-4-O-benzyl-1-β-D-galactopyranosyl]-(1→4)-[3,6-di-O-benzyl-2-deoxy-2-(2,2,2-trichloroethoxy)carbonylamino-β-D-glucopyranosyl]-(1→2)-3,4,6-tri-O-benzyl-α-D-mannopyranoside 9. A mixture of acceptor 8 (1.04 g, 1.03 mmol, 1 equiv.), donor 4 (1.39 g, 1.29 mmol, 1.25 equiv.) and activated pulverized 4 Å MS (0.70 g) in anhydrous CH₂Cl₂ (7 mL) was stirred under argon for 1 h. Then, the reaction mixture was cooled to −40° C. and NIS (465 mg, 2.06 mmol, 2 equiv.) was added, followed by TfOH (0.5 M in Et₂O, 0.62 mL, 0.31 mmol, 0.3 equiv.) with stirring. Upon completion of the reaction (TLC indicated the disappearance of starting materials after ˜2 hrs), the reaction mixture was quenched with Et₃N (0.4 mL) and filtered through a pad of Celite. The filtrate was diluted with CH₂Cl₂ (30 mL), washed with 20% aq. Na₂S₂O₃ (10 mL), satd. aq. NaHCO₃ (15 mL) and brine (8 mL). The separated organic layer was dried over MgSO₄, and concentrated. The obtained residue was purified by silica gel column chromatography using acetone/toluene (1:2) as eluent to give compound 9 as a white powder (1.30 g, 64%). R_{f}=0.51 (silica gel, acetone: toluene=2:3); ¹H NMR (600 MHz, CDCl₃): δ 7.90-7.88 (m, 4H, Ar—H), 7.48-7.45 (m, 2H, Ar—H), 7.34-7.22 (m, 24H, Ar—H), 7.18-7.13 (m, 10H, Ar—H), 5.85-5.78 (m, 2H), 5.47-5.44 (m, 1H), 5.35-5.32 (m, 4H), 5.24-5.16 (m, 2H), 5.11 (dd, J=10.2, 1.2 Hz, 1H), 5.06-4.97 (m, 3H, C1-H_{β}), 4.85-4.73 (m, 4H, C1-H_{α}, C1-H_{β}), 4.70-4.54 (m, 6H), 4.53-4.42 (m, 4H), 4.33-4.26 (m, 2H), 4.23-4.18 (m, 3H), 4.14-4.04 (m, 3H), 3.97-3.93 (m, 1H), 3.89-3.81 (m, 5H), 3.73-3.58 (m, 10H), 3.45-3.43 (m, 1H), 3.32-3.30 (m, 1H), 2.16 (s, 3H, —CH₃), 2.13 (s, 3H, —CH₃), 2.10 (s, 3H, —CH₃), 2.01 (s, 3H, —CH₃), 1.92 (s, 3H, —CH₃); ¹³C NMR (150 MHz, CDCl₃): δ 170.7, 170.6, 170.4, 170.2, 169.8, 165.6, 165.4, 165.4, 165.1, 153.8, 138.8, 138.6, 138.5, 138.2, 138.2, 138.1, 133.7, 133.2, 133.1, 133.1, 130.8, 130.0, 129.8, 129.6, 129.4, 129.0, 128.4, 128.3, 128.3, 128.2, 128.2, 128.2, 128.1, 128.1, 128.0, 128.0, 127.9, 127.6, 127.6, 127.5, 127.4, 127.3, 127.2, 125.2, 117.1, 100.0, 98.3, 98.2, 96.9, 95.6, 88.1, 86.8, 77.9, 75.0, 74.7, 74.6, 74.5, 74.2, 74.1, 73.9, 73.4, 73.3, 73.1, 73.0, 72.4, 71.9, 71.4, 70.9, 70.9, 69.5, 69.2, 69.1, 69.0, 68.0, 67.9, 67.2, 62.9, 62.3, 57.2, 53.2, 45.5, 23.3, 21.0, 20.7, 20.7, 20.6; ¹⁹F NMR (376 MHz, CDCl₃): δ −215.6; HRMS (ESI-TOF) m/e: Calcd for C₁₀₀H₁₀₈C₁₃FN₂O₃₁Na [M+Na]⁺: 1979.5878 found 1979.5889.

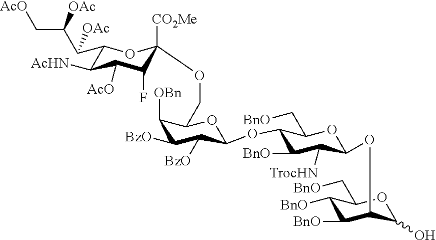

[Methyl 5-acetamido-4,7,8,9-tetra-O-acetyl-3,5-dideoxy-3-fluoro-D-erythro-α-L-manno-non-2-ulopyranosonate]-(2→6)-[2,3-di-O-benzoyl-4-O-benzyl-1-β-D-galactopyranosyl]-(1→4)-[3,6-di-O-benzyl-2-deoxy-2-(2,2,2-trichloroethoxy)carbonylamino-β-D-glucopyranosyl]-(1→2)-3,4,6-tri-O-benzyl-α-D-mannopyranoside 10. To a stirred solution of tetrasaccharide 9 (578 mg, 0.30 mmol, 1 equiv.) in acetic acid (6.0 mL, acetic acid/water, 10:1=v/v) was added CH₃COONa (121 mg, 1.48 mmol, 5 equiv.) followed by addition of PdCl₂ (105 mg, 0.59 mmol, 2 equiv.) at room temperature. After 20 h, when TLC indicated the disappearance of starting material, the reaction mixture was diluted with ethyl acetate (20 mL) and poured into satd. aq. NaHCO₃ (20 mL). The aqueous layer was extracted with ethyl acetate (2×10 mL), and the combined organic phases were dried over MgSO₄, and concentrated. The obtained residue was purified by silica gel column chromatography using acetone/hexane (2:3) as eluent to give compound 10 as a white powder (465 mg, 82%). R_{f}=0.23 (silica gel, acetone:hexane=2:3); ¹H NMR (600 MHz, CDCl₃): δ 7.90-7.87 (m, 4H, Ar—H), 7.48-7.45 (m, 2H, Ar—H), 7.34-7.20 (m, 24H, Ar—H), 7.19-7.12 (m, 10H, Ar—H), 5.80-5.75 (m, 1H), 5.45-5.43 (m, 1H), 5.33-5.28 (m, 3H), 5.21-4.89 (m, 5H, man-C1-H_{α}, man-C1-H_{β}, C1-H_{β}), 4.84-4.30 (m, 15H, C1-H_{β}), 4.27-3.79 (m, 12H), 3.78-3.43 (m, 12H), 2.13-2.12 (m, 6H, -2CH₃), 2.09-2.08 (m, 3H, —CH₃), 2.02-2.00 (m, 3H, —CH₃), 1.91 (br, 3H, —CH₃); ¹³C NMR (150 MHz, CDCl₃): 8170.8, 170.7, 170.7, 170.7, 170.6, 170.5, 170.4, 170.3, 170.3, 170.2, 169.9, 169.8, 165.6, 165.6, 165.4, 165.2, 165.2, 154.0, 138.6, 138.5, 138.3, 138.2, 138.1, 133.2, 133.1, 129.8, 129.6, 129.5, 129.0, 128.4, 128.4, 128.3, 128.3, 128.2, 128.2, 128.2, 128.1, 128.0, 127.9, 127.9, 127.7, 127.7, 127.6, 127.5, 127.5, 127.4, 127.4, 127.3, 100.0, 99.5, 98.3, 98.2, 98.1, 95.8, 95.7, 92.3, 88.2, 88.1, 86.8, 76.1, 74.8, 74.7, 74.6, 74.6, 74.3, 74.2, 74.2, 73.9, 73.8, 73.3, 73.2, 73.1, 73.1, 72.7, 72.6, 72.4, 72.3, 71.6, 71.4, 71.2, 70.9, 70.8, 69.9, 69.5, 69.3, 69.2, 69.1, 69.0, 68.1, 68.0, 67.2, 67.1, 63.0, 62.3, 62.2, 62.1, 53.3, 53.3, 45.5, 45.4, 26.3, 23.3, 21.1, 20.8, 20.7, 20.7, 20.6, 20.6; ¹⁹F NMR (376 MHz, CDCl₃): δ −215.2, −215.7; HRMS (ESI-TOF) m/e: Calcd for C₉₇H₁₀₄Cl₃FN₂O₃₁Na [M+Na]⁺: 1939.5565 found 1939.5619.

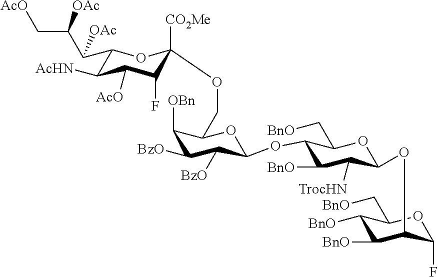

[Methyl 5-acetamido-4,7,8,9-tetra-O)-acetyl-3,5-dideoxy-3-fluoro-D-erythro-α-L-manno-non-2-ulopyranosonate]-(2→6)-[2,3-di-O)-benzoyl-4-O-benzyl-1-β-D-galactopyranosyl]-(1→4)-[3,6-di-O-benzyl-2-deoxy-2-(2,2,2-trichloroethoxy)carbonylamino-β-D-glucopyranosyl]-(1→2)-3,4,6-tri-O-benzyl-α-D-mannopyranosyl fluoride 11. To a well-stirred solution of hemiacetal 10 (395 mg, 0.21 mmol, 1 equiv.) in anhydrous CH₂Cl₂ (12 mL) was added DAST (81.6 μL, 0.62 mmol, 3 equiv.) at −20° C. The reaction mixture was vigorously stirred until TLC indicated the disappearance of starting material (3 h). The reaction mixture was diluted with CH₂Cl₂ (10 mL) and washed with satd. aq. NaHCO3 (5 mL) and brine (4 mL). The organic phase was dried over MgSO₄, filtered, and concentrated. The obtained residue was purified by silica gel column chromatography using acetone/hexane (3:4) as eluent to give compound 11 as a white powder (289 mg, 73%). R_{f}=0.32 (silica gel, acetone:hexane=3:4); ¹H NMR (600 MHz, CDCl₃): δ 7.89-7.88 (m, 4H, Ar—H), 7.47-7.45 (m, 2H, Ar—H), 7.33-7.22 (m, 22H, Ar—H), 7.17-7.12 (m, 12H, Ar—H), 5.78 (dd, J=10.2, 7.8 Hz, 1H), 5.53-5.40 (m, 2H), 5.33-5.29 (m, 3H, C1-H_{α}), 5.21 (dd, J=27.6, 11.4 Hz, 1H, sia-C4-H), 5.05-4.96 (m, 2H, C1-H_{β}), 4.94 (d, J=11.4 Hz, 1H), 4.82-4.79 (m, 4H, C1-H_{β}), 4.75-4.72 (m, 1H), 4.68-4.59 (m, 3H), 4.58-4.5 4(m, 2H), 4.50-4.48 (m, 2H), 4.30 (dd, J=12.6, 3.0 Hz, 1H), 4.26 (d, J=10.2 Hz, 1H), 4.23-4.15 (m, 4H), 4.08-4.04 (m, 2H), 3.95 (dd, J=9.0, 8.4 Hz, 1H), 3.89-3.78 (m, 5H), 3.72-3.57 (m, 9H), 3.50-3.48 (m, 1H), 3.35 (d, J=0.6 Hz, 1H) 2.14 (s, 3H, —CH₃), 2.12 (s, 3H, —CH₃), 2.09 (s, 3H, —CH₃), 2.00 (s, 3H, —CH₃), 1.91 (s, 3H, —CH₃); ¹³C NMR (150 MHz, CDCl₃): δ 170.8, 170.7, 170.4, 170.2, 169.9, 165.6, 165.4, 165.2, 153.9, 138.2, 138.1, 137.8, 133.2, 133.1, 129.8, 129.6, 129.4, 129.0, 128.4, 128.3, 128.2, 128.1, 128.0, 127.9, 127.7, 127.6, 127.6, 127.4, 127.3, 127.3, 106.9, 105.4, 99.8, 99.2, 98.3, 98.2, 95.6, 88.1, 86.8, 74.9, 74.8, 74.2, 74.0, 73.9, 73.8, 73.3, 73.2, 72.5, 71.4, 71.3, 70.9, 69.1, 69.1, 68.9, 68.0, 67.3, 62.8, 62.3, 56.9, 53.3, 45.6, 23.4, 21.1, 20.8 (2 C), 20.6; ¹⁹F NMR (376 MHz, CDCl₃): δ −138.6, −215.7; HRMS (ESI-TOF) m/e: Calcd for C₉₇H₁₀₃C₁₃F₂N₂O₃₀Na [M+Na]⁺: 1941.5521 found 1941.5521.

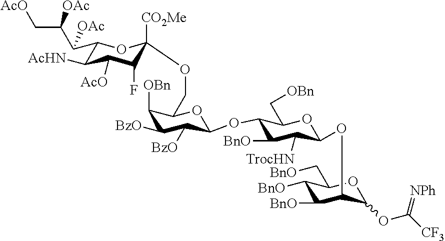

(N-Phenyl)-2,2,2-trifluoroacetimidate [methyl 5-acetamido-4,7,8,9-tetra-O-acetyl-3,5-dideoxy-3-fluoro-D-erythro-α-L-manno-non-2-ulopyranosonate]-(2→6)-[2,3-di-O-benzoyl-4-O-benzyl-1-β-D-galactopyranosyl]-(1→4)-[3,6-di-O-benzyl-2-deoxy-2-(2,2,2-trichloroethoxy)carbonylamino-β-D-glucopyranosyl]-(1→2)-3,4,6-tri-O-benzyl-D-mannopyranoside 12. To a well-stirred solution of hemiacetal 10 (395 mg, 0.21 mmol, 1 equiv.) in anhydrous CH₂Cl₂ (12 mL) was added Cs₂CO₃ (83.2 mg, 0.26 mmol, 2 equiv.) and 2,2,2-trifluoro-N-phenyl-acetimidoyl chloride (41 μL, 0.26 mmol, 2 equiv.) at 0° C. with stirring, and then the mixture was warmed up to room temperature and stirred for 3 h. The reaction mixture was diluted with CH₂Cl₂ (10 mL), filtered through a pad of Celite, and concentrated. The obtained residue was purified by silica gel column chromatography using acetone/hexane (1:2) as eluent to give compound 12 as a white foam (150 mg, 56%); anomeric mixture (α:β=1:1). R_{f}=0.55 (silica gel, acetone:hexane=1:1); ¹H NMR (600 MHz, CDCl₃): δ 7.93-7.88 (m, 4H, Ar—H), 7.49-7.45 (m, 2H, Ar—H), 7.40-7.12 (m, 38H, Ar—H), 6.75-6.71 (m, 1H, Ar—H), 5.82-5.78 (m, 1H), 5.46-5.42 (m, 1H), 5.36-5.30 (m, 3H), 5.24-5.16 (m, 1H), 5.08-4.81 (m, 5H, 0.5man-C1-H_{α}, 2C1-H_{β}), 4.78-4.74 (m, 1H), 4.69-4.41 (m, 9H, 0.5man-C1-H_{β}), 4.33-4.06 (m, 7H), 3.95-3.82 (m, 5H), 3.69-3.36 (m, 10H), 2.16-2.10 (m, 9H, -3CH₃), 2.01-1.99 (m, 3H, —CH₃), 1.92 (s, 3H, —CH₃); ¹³C NMR (150 MHz, CDCl₃): δ 170.7, 170.7, 170.7, 170.7, 170.4, 170.2, 169.9, 169.9, 165.6, 165.6, 165.4, 165.2, 165.1, 154.1, 153.9, 143.3, 143.1, 138.7, 138.5, 138.3, 138.2, 138.2, 138.1, 138.0, 138.0, 137.8, 137.7, 133.2, 133.1, 133.1, 129.8, 129.6, 129.4, 129.3, 129.0, 128.7, 128.4, 128.4, 128.3, 128.3, 128.3, 128.2, 128.2, 128.1, 128.1, 128.1, 128.0, 127.9, 127.7, 127.7, 127.6, 127.5, 127.5, 127.5, 127.4, 127.3, 119.4, 119.2, 102.3, 100.2, 100.0, 98.3, 98.2, 95.6, 95.5, 88.0, 86.7, 80.0, 79.3, 75.9, 75.0, 74.7, 74.7, 74.5, 74.3, 74.2, 73.8, 73.8, 73.5, 73.3, 73.2, 73.1, 72.8, 72.4, 72.3, 71.5, 71.4, 71.1, 71.0, 70.9, 70.1, 69.8, 69.3, 69.1, 68.2, 67.2, 67.2, 62.8, 62.3, 62.2, 53.3, 53.2, 45.5, 23.3, 21.0, 21.0, 20.7, 20.7, 20.6; ¹⁹F NMR (376 MHz, CDCl₃): δ −65.2, −65.4, −65.5, −65.6, −65.6, −65.7, −215.6, −215.7; HRMS (ESI-TOF) m/e: Calcd for C₁₀₅H₁₀₈C₁₃F₄N₃O₃₁Na [M+Na]⁺: 2110.5860 found 2110.5865.

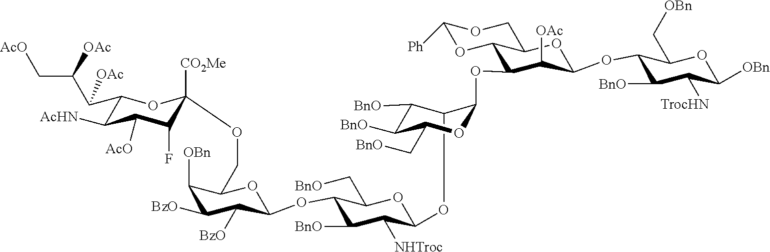

Benzyl [methyl 5-acetamido-4,7,8,9-tetra-O-acetyl-3,5-dideoxy-3-fluoro-D-erythro-α-L-manno-non-2-ulopyranosonate]-(2→6)-[2,3-di-O-benzoyl-4-O-benzyl-1-β-D-galactopyranosyl]-(1→4)-[3,6-di-O-benzyl-2-deoxy-2-(2,2,2-trichloroethoxy)carbonylamino-β-D-glucopyranosyl]-(1→2)-[3,4,6-tri-O-benzyl-α-D-mannopyranosyl]-(1→3)-[2-O-acetyl-4,6-O-benzylidine-β-D-mannopyranosyl]-(1→4)-3,6-di-O-benzyl-2-deoxy-2-(2,2,2-trichloroethoxy)carbonylamino-β-D-glucopyranoside 14. A mixture AgOTf (194 mg, 0.75 mmol, 6 equiv. with respect to acceptor), bis Cp₂HfCl₂ (167 mg, 0.44 mmol, 3.5 equiv. with respect to acceptor), and freshly activated 4 Å MS (1.50 g) in anhydrous toluene (15 mL) was stirred at room temperature under argon for 1 h. Then, the reaction mixture was cooled to −20° C. and treated with a solution of donor 11 (290 mg, 0.15 mmol, 1.2 equiv.) and acceptor 13 (115 mg, 0.13 mmol, 1 equiv.) in anhydrous toluene (8 mL). The reaction mixture was stirred at 0° C. until TLC indicated the disappearance of starting material (3 hrs). Upon completion of the reaction, the reaction mixture was quenched with Et₃N (0.25 mL), diluted with EtOAc (25 mL), and filtered through a pad of Celite. The filtrate was washed twice with satd. aq. NaHCO₃ (10 mL) and brine (6 mL), and the organic phase was dried over MgSO₄, filtered, and concentrated. The obtained residue was purified by silica gel column chromatography using acetone/toluene (1:2) as eluent to give compound 14 as a white powder (299 mg, 85%).

One-pot Synthesis: A mixture of acceptor 18 (40.0 mg, 0.037 mmol, 1 equiv.), donor 4 (60.1 mg, 0.056 mmol, 1.5 equiv.) and activated pulverized 4 Å MS (0.25 g) in anhydrous CH₂Cl₂ (1.25 mL) was stirred under argon for 1 h. Then, the reaction mixture was cooled to −40° C. and NIS (12.6 mg, 0.056 mmol, 1.5 equiv.) was added, followed by TfOH (0.5 M in Et₂O, 22.3 μL, 0.011 mmol, 0.3 equiv.) and the mixture was then allowed to stir to −20° C. for 2 h. Next, upon disappearance of starting materials (monitored by TLC), the reaction mixture was treated sequentially with acceptor 13 (34.2 mg, 0.037 mmol, 1 equiv.), NIS (16.8 mg, 0.075 mmol, 2 equiv.) and TfOH (0.5 M in Et20, 22.3 μL, 0.011 mmol, 0.3 equiv.) in anhydrous CH₂Cl₂ (1.25 mL) with stirring for 10 min under argon. Upon addition of all the reagents, the mixture was allowed to warm up to −10° C. and continued to stir until TLC indicated the disappearance of starting materials (1 h). Upon completion, the reaction was quenched with Et₃N (0.4 mL) and filtered through a pad of Celite. The filtrate was diluted with CH₂Cl₂ (10 mL), washed with 20% aq. Na₂S₂O₃ (3 mL), satd. aq. NaHCO₃ (2 mL) and brine (2 mL). The separated organic layer was dried over MgSO₄, filtered and concentrated in vacuo. The obtained residue was purified by silica gel column chromatography using acetone/toluene (1:2) as eluent, followed by a second column chromatography using acetone/hexane (3:4) as eluent to give compound 14 as a white powder (27 mg, 26%). R_{f}=0.43 (silica gel, acetone: toluene=3:5); ¹H NMR (600 MHz, CDCl₃): δ 7.96-7.95 (m, 2H, Ar—H), 7.91-7.90 (m, 2H, Ar—H), 7.51-7.47 (m, 2H, Ar—H), 7.36-7.26 (m, 35H, Ar—H), 7.23-7.10 (m, 19H, Ar—H), 5.84 (dd, J=10.2, 7.8 Hz, 1H), 5.45-5.42 (m, 1H), 5.34-5.27 (m, 4H), 5.21-5.15 (m, 2H), 5.04-4.94 (m, 4H, C1-H_{α}, C1-H_{β}), 4.87-4.78 (m, 4H), 4.71-4.50 (m, 14H, 2C1-H_{β}), 4.46-4.34 (m, 5H), 4.31-4.21 (m, 4H), 4.16 (dd, J=12.6, 5.4 Hz, 1H), 4.13-4.06 (m, 2H), 3.98-3.95 (m, 4H, C1-H_{β}), 3.84-3.57 (m, 17H), 3.51-3.41 (m, 4H), 3.36-3.35 (m, 2H), 3.15 (br, 1H), 2.93 (br, 1H), 2.68-2.67 (m, 1H), 2.12 (s, 3H, —CH₃), 2.11 (s, 3H, —CH₃), 2.09 (s, 3H, —CH₃), 1.99 (s, 3H, —CH₃), 1.91 (s, 3H, —CH₃), 1.85 (s, 3H, —CH₃); ¹³C NMR (150 MHz, CDCl₃): δ170.8, 170.7, 170.4, 170.2, 169.7, 169.4, 165.6, 165.4, 165.2, 153.7, 153.6, 138.9, 138.7, 138.4, 138.3, 137.9, 137.1, 133.2, 129.8, 129.7, 129.5, 129.1, 128.9, 128.6, 128.5, 128.4, 128.4, 128.3, 128.3, 128.2, 128.1, 128.1, 128.0, 128.0, 127.9, 127.9, 127.9, 127.8, 127.8, 127.6, 127.6, 127.6, 127.5, 127.4, 127.4, 127.3, 127.2, 126.3, 102.1, 100.1, 99.0, 98.7, 98.6, 95.8, 95.5, 88.2, 86.9, 78.8, 78.4, 74.8, 74.5, 74.3, 74.2, 73.9, 73.8, 73.3, 72.9, 72.3, 71.9, 71.4, 71.1, 70.7, 70.3, 69.2, 69.1, 69.0, 68.4, 68.3, 67.9, 67.2, 66.2, 63.2, 62.3, 57.4, 56.8, 53.3, 45.5, 23.3, 21.0, 20.8, 20.7, 20.6, 20.6; ¹⁹F NMR (376 MHz, CDCl₃): 67 −215.6; HRMS (ESI-TOF) m/e: Calcd for C₁₄₂H₁₅₀C₁₆FN₃O₄₃Na₂ [M+2Na]²⁺: 1429.8771 found 1429.8864.

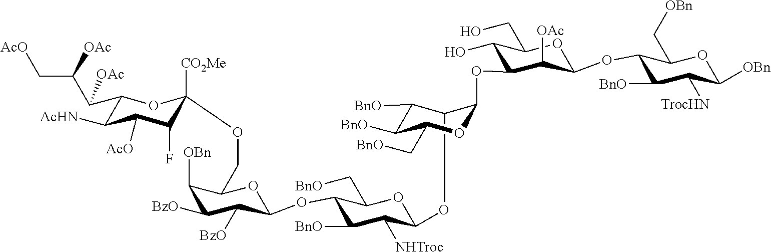

Benzyl [methyl 5-acetamido-4,7,8,9-tetra-O-acetyl-3,5-dideoxy-3-fluoro-D-erythro-α-L-manno-non-2-ulopyranosonate]-(2→6)-[2,3-di-O-benzoyl-4-O-benzyl-1-β-D-galactopyranosyl]-(1→4)-[3,6-di-O-benzyl-2-deoxy-2-(2,2,2-trichloroethoxy)carbonylamino-β-D-glucopyranosyl]-(1→2)-[3,4,6-tri-O-benzyl-α-D-mannopyranosyl]-(1→3)-[2-O-acetyl-β-D-mannopyranosyl]-(1→4)-3,6-di-O-benzyl-2-deoxy-2-(2,2,2-trichloroethoxy)carbonylamino-β-D-glucopyranoside 15. To a stirred solution of starting material 14 (318 mg, 0.11 mmol, 1 equiv.) in acetonitrile (12 mL) was added p-TsOH·H₂O (21.5 mg, 0.11 mmol, 1 equiv.). The reaction mixture was vigorously stirred until TLC indicated the disappearance of starting material (6 hrs). Upon completion, the reaction mixture was quenched with Et₃N (0.3 mL) and concentrated under high vacuum. The obtained residue was purified by silica gel column chromatography using acetone/toluene (3:5) as eluent to give compound 15 as a white powder (231 mg, 75%). R_{f}=0.25 (silica gel, acetone: toluene=1:2); ¹H NMR (600 MHz, CDCl₃): δ 7.88-7.87 (m, 4H, Ar—H), 7.46 (dd, J=7.2, 7.2 Hz, 1H, Ar—H), 7.43 (dd, J=7.2, 7.2 Hz, 1H, Ar—H), 7.33-7.13 (m, 47H, Ar—H), 7.09-7.08 (m, 2H, Ar—H), 6.01 (br, 1H), 5.76 (dd, J=10.2, 7.8 Hz, 1H), 5.46-5.43 (m, 1H), 5.38 (d, J=8.4 Hz, 1H), 5.31-5.29 (m, 3H), 5.19-5.13 (m, 2H, C1-H_{α}), 5.05-4.96 (m, 3H, C1-H_{β}), 4.89-4.79 (m, 4H, C1-H_{β}), 4.69-4.60 (m, 8H, C1-H_{β}), 4.57-4.49 (m, 8H, C1-H_{β}), 4.45 (d, J=12.0 Hz, 1H), 4.40-4.36 (m, 2H), 4.31 (dd, J=12.6, 2.4 Hz, 1H), 4.24-4.16 (m, 4H), 4.12-4.09 (m, 2H), 4.02 (br, 1H), 3.98-3.93 (m, 2H), 3.91-3.86 (m, 2H), 3.80 (dd, J=7.8, 6.0 Hz, 1H), 3.76-3.69 (m, 6H), 3.66-3.37 (m, 18H), 2.96-2.93 (m, 1H), 2.13 (s, 3H, —CH₃), 2.11 (s, 3H, —CH₃), 2.09 (s, 3H, —CH₃), 1.98 (s, 3H, —CH₃), 1.93 (s, 3H, —CH₃), 1.90 (s, 3H, —CH₃); ¹³C NMR (150 MHz, CDCl₃): δ 170.8, 170.7, 170.4, 170.3, 170.2, 169.8, 165.6, 165.5, 165.3, 154.3, 153.8, 138.6, 138.4, 138.3, 138.1, 138.0, 137.8, 137.2, 133.3, 133.1, 129.8, 129.6, 129.4, 129.0, 128.5, 128.4, 128.3, 128.2, 128.2, 127.9, 127.9, 127.8, 127.7, 127.6, 127.5, 127.5, 127.4, 127.4, 127.3, 100.1, 99.1, 98.3, 98.2, 98.1, 95.7, 95.5, 88.2, 86.9, 78.6, 78.2, 75.4, 75.1, 74.8, 74.7, 74.4, 74.2, 74.1, 73.8, 73.3, 73.2, 72.6, 71.9, 71.8, 71.5, 71.4, 70.9, 70.6, 69.5, 62.3, 57.2, 56.8, 53.8, 53.3, 45.4, 23.3, 21.1, 21.0, 20.7 (2C), 20.7; ¹⁹F NMR (376 MHz, CDCl₃): δ −215.6; HRMS (ESI-TOF) m/e: Calcd for C₁₃₅H₁₄₆Cl₆FN₃O₄₃Na [M+Na]⁺: 2748.7337 found 2748.7373.

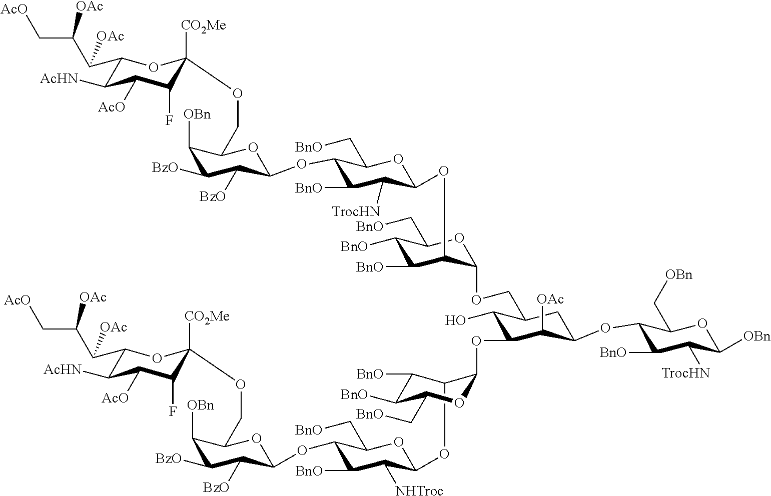

Benzyl [methyl 5-acetamido-4,7,8,9-tetra-O-acetyl-3,5-dideoxy-3-fluoro-D-erythro-α-L-manno-non-2-ulopyranosonate-(2→6)-2,3-di-O-benzoyl-4-O-benzyl-1-β-D-galactopyranosyl-(1→4)-3,6-di-O-benzyl-2-deoxy-2-(2,2,2-trichloroethoxy)carbonylamino-β-D-glucopyranosyl-(1→2)-3,4,6-tri-O-benzyl-α-D-mannopyranosyl-(1→3)]-[methyl 5-acetamido-4,7,8,9-tetra-O-acetyl-3,5-dideoxy-3-fluoro-D-erythro-α-L-manno-non-2-ulopyranosonate-(2→6)-2,3-di-O-benzoyl-4-O-benzyl-1-β-D-galactopyranosyl-(1→4)-3,6-di-O-benzyl-2-deoxy-2-(2,2,2-trichloroethoxy)carbonylamino-β-D-glucopyranosyl-(1→2)-3,4,6-tri-O-benzyl-α-D-mannopyranosyl-(1→6)]-[2-O-acetyl-β-D-mannopyranosyl]-(1→4)-3,6-di-O-benzyl-2-deoxy-2-(2,2,2-trichloroethoxy)carbonylamino-β-D-glucopyranoside 16.

From fluoride donor: A mixture of AgOTf (142 mg, 0.55 mmol, 8 equiv. with respect to acceptor), Cp₂HfCl₂ (105 mg, 0.28 mmol, 4 equiv. with respect to acceptor), and freshly activated 4 Å MS (1 g) in anhydrous toluene (10 mL) was stirred at room temperature under argon atmosphere for 1 h. Then, the reaction mixture was cooled to −40° C. and a solution of donor 11 (232 mg, 0.12 mmol, 1.75 equiv.) and acceptor 15 (188 mg, 0.069 mmol, 1 equiv.) in anhydrous toluene (1 mL) was added. Stirring was continued at −15° C. for 3 h. The reaction mixture was quenched with Et₃N (0.20 mL), diluted with EtOAc (20 mL), and filtered through a pad of Celite. The filtrate was washed twice with satd. aq. NaHCO₃ (8 mL) and brine (4 mL). The organic phase was dried over MgSO₄, filtered and concentrated in vacuo. The obtained residue was purified by silica gel column chromatography using acetone/toluene (2:3) as eluent to give compound 16 as a white powder (223 mg, 70%) along with the recovered 15 (23.0 mg, 80% brsm).

From trifluoroacetimidate donor: A mixture of acceptor 15 (32.0 mg, 0.012 mmol, 1 equiv.), donor 12 (42.9 mg, 0.021 mmol, 1.75 equiv.) and activated pulverized 4 Å MS (200 mg) in anhydrous CH₂Cl₂ (2 mL) was stirred under argon for 30 min. Then, the reaction mixture was cooled to −60° C. and treated with TfOH (0.5 M in Et₂O, 5.86 μL, 2.93 μmol, 0.25 equiv. with respect to acceptor). After stirring at −20° C. for 3 h. the reaction mixture was quenched with Et₃N (0.10 mL), diluted with CH₂Cl₂ (10 mL), and filtered through a pad of Celite. The filtrate was washed twice with satd. aq. NaHCO3 (4 mL) and brine (3 mL). The organic phase was dried over MgSO₄, filtered, and concentrated. The obtained residue was purified by silica gel column chromatography using acetone/toluene (2:3) as eluent to give compound 16 as a white powder (18.0 mg, 33%) along with the recovered acceptor (12.6 mg, 55% brsm). R_{f}=0.17 (silica gel, acetone: toluene=1:2); ¹H NMR (600 MHz, CDCl₃): δ 7.91-7.86 (m, 8H, Ar—H), 7.48-7.42 (m, 4H, Ar—H), 7.33-7.11 (m, 83H, Ar—H), 5.81-5.75 (m, 2H), 5.43-5.38 (m, 3H), 5.31-5.07 (m, 11H, 2C1-H_{α}), 5.03-4.78 (m, 9H, 2C1-H_{β}), 4.75-4.08(m, 48H, 4C1-H_{β}), 4.05-3.89 (m, 7H), 3.87-2.99 (m, 40H), 2.13-2.12 (m, 6H, -2CH₃), 2.10-2.07 (m, 15H, -5CH₃), 1.99-1.97 (s, 6H, -2CH₃), 1.90-1.88 (m, 6H, -2CH₃); ¹³C NMR (150MHz, CDCl₃): δ 170.7, 170.6, 170.6, 170.4, 170.2, 169.8, 169.7, 169.6, 165.6, 165.5, 165.4, 165.4, 165.1, 154.0, 153.6, 139.1, 138.8, 138.4, 138.3, 138.1, 138.0, 137.7, 133.2, 133.1, 129.8, 129.8, 129.7, 129.6, 129.6, 129.5, 129.5, 129.1, 129.1, 128.4, 128.4, 128.3, 128.2, 128.2, 128.0, 128.0, 127.8, 127.8, 127.8, 127.7, 127.7, 127.5, 127.4, 127.4, 127.3, 127.2, 127.1, 100.1, 99.8, 98.8, 98.4, 98.2, 95.7, 88.1, 86.8, 78.3, 77.8, 75.3, 74.7, 74.5, 74.4, 74.3, 74.2, 74.0, 73.9, 73.4, 73.3, 73.3, 73.2, 73.1, 73.0, 72.9, 72.5, 72.4, 72.0, 71.9, 71.5, 71.0, 71.0, 70.9, 70.9, 70.7, 69.5, 69.4, 69.2, 69.1, 69.0, 69.0, 68.5, 68.1, 68.1, 68.0, 67.2, 62.9, 62.2, 57.5, 57.2, 53.8, 53.2, 53.2, 45.5, 45.3, 23.3, 21.0, 21.0, 20.8, 20.7, 20.7, 20.6; ¹⁹F NMR (376 MHz, CDCl₃): δ −215.6 (2F); HRMS (ESI-TOF) m/e: Calcd for C₂₃₂H₂₅₅C₁₉F₂N₆O₇₄ [M+H₃O+NH₄]²⁺: 2330.6770 found 2330.6724.

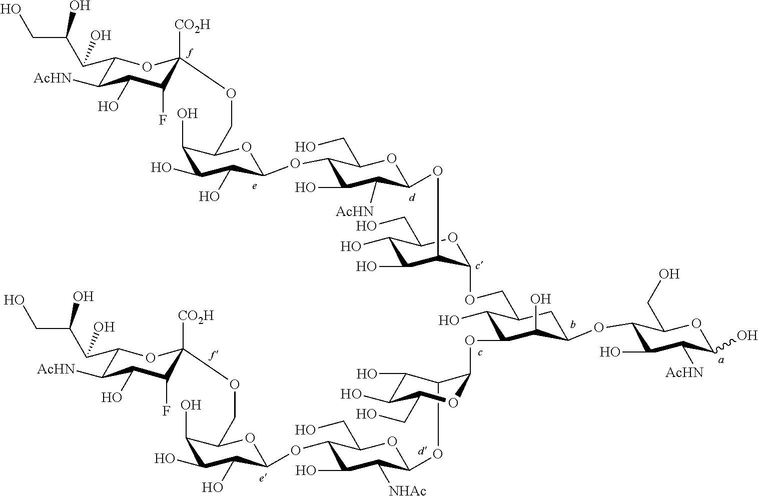

[5-Acetamido-3,5-dideoxy-3-fluoro-D-erythro-α-L-manno-non-2-ulopyranosonate-(2→6)-β-D-galactopyranosyl-(1→4)-2-acetamido-2-deoxy-β-D-glucopyranosyl-(1→2)-α-D-mannopyranosyl-(1→3)]-[5-acetamido-3,5-dideoxy-3-fluoro-D-erythro-α-L-manno-non-2-ulopyranosonate-(2→6)-β-D-galactopyranosyl-(1→4)-2-acetamido-2-deoxy-β-D-glucopyranosyl-(1→2)-α-D-mannopyranosyl-(1→6)]-[β-D-mannopyranosyl]-(1→4)-2-acetamido-2-deoxy-D-glucopyranoside 17. A solution of protected glycan 16 (103 mg, 0.022 mmol, 1 equiv.) and LiOH (51.5 mg, 50% by S.M. wt) in a mixture of 1,4-dioxane/H₂O (3 mL, 4:1=v/v) was stirred at 90° C. for 16 h. The reaction mixture was concentrated under high vacuum and subjected to the acetylation conditions (pyridine (2.5 mL), Ac2O (1.5 mL), r.t., 16 h). After the solvent was removed, the crude residue was purified by LiChroprep® RP-18 reverse-phase column chromatography using H₂O/MeOH (1:5) as eluent. The product was deacetylated by stirring with NaOMe in MeOH (3 mL, 0.5 M) for 16 h. The reaction mixture was neutralized with IR-120, filtered, and concentrated in vacuo. The residue was purified by LiChroprep® RP-18 reverse-phase column chromatography using H₂O/MeOH (1:4) as eluent. The crude product of deacetylation was dissolved in a mixture of MeOH/H₂O/HCOOH (3 mL, 6:3:1=v/v/v) and treated with Pd(OH)₂ (51.5 mg, 50% by S.M. wt) for 20 h. The reaction mixture was filtered through a pad of Celite and concentrated in vacuo. The residue was purified by (BIO-RAD) Biogel P-2 column chromatography (eluted with water), followed by purification with LiChroprep® RP-18 reverse-phase column chromatography (eluted with water) to give compound 17 as a white powder (18.3 mg, 40%) of anomeric mixture (α:β=0.65:0.35). ¹H NMR (600 MHz, D₂O): δ 5.23 (d, J=3.0 Hz, 0.65H, a-C1-H_{α}), 5.14 (d, J=52.8 Hz, 2H, sia-C3-H), 5.14 (br, 1H, c-C1-H_{α}), 4.96 (br, 1H, c′-C1-H_{α}), 4.79 (br, 1H, b-C1-H), 4.73 (br, 0.35H, a-C1-H_{β}), 4.60 (d, J=7.8 Hz, 2H, dd'-C1-H_{β}), 4.48 (d, J=7.8 Hz, 2H, ee'-C1-H3), 4.28-4.21 (m, 4H), 4.13 (br, 1H), 4.00-3.51 (m, 57H), 2.09-2.08 (m, 9H, -3CH₃), 2.05 (br, 6H, -2CH₃); ¹³C NMR (150 MHz, D₂O): δ 174.5, 174.2, 174.1, 102.7, 102.6, 100.0, 99.1, 98.9, 98.9, 98.7, 98.6, 96.6, 94.4, 91.0, 90.0, 89.8, 80.0, 79.7, 79.3, 78.9, 76.0, 75.8, 74.1, 74.0, 73.9, 73.1, 72.9, 72.7, 72.4, 71.9, 71.9, 71.6, 71.5, 71.1, 70.3, 69.8, 69.4, 69.2, 69.1, 69.0, 68.7, 68.0, 67.8, 66.9, 66.8, 65.4, 65.2, 63.6, 62.2, 61.2, 61.2, 59.7, 59.6, 55.6, 54.4, 53.1, 46.3, 21.9, 21.6; ¹⁹F NMR (376 MHz, D₂O): δ −217.4, −217.4; HRMS (ESI-TOF) m/e: Calcd for C₇₆H₁₂₁F₂N₅O₅₇ [M−2H]⁻² :1026.8351 found 1026.8299.

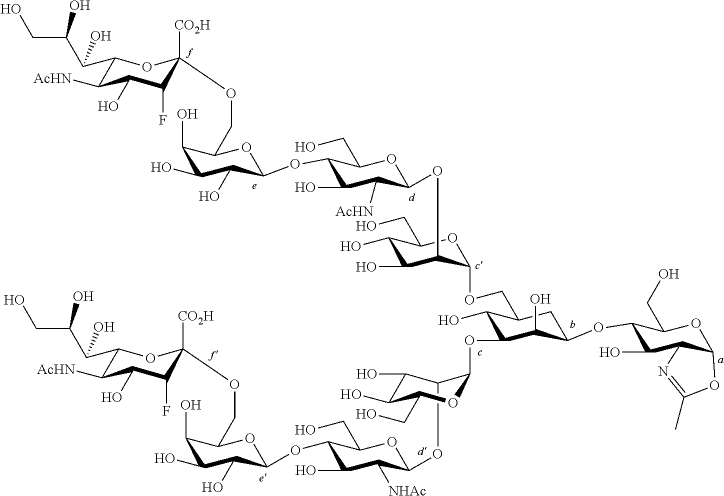

[5-Acetamido-3,5-dideoxy-3-fluoro-D-erythro-α-L-manno-non-2-ulopyranosonate-(2→6)-β-D-galactopyranosyl-(1→4)-2-acetamido-2-deoxy-β-D-glucopyranosyl-(1→2)-α-D-mannopyranosyl-(1→3)]-[5-acetamido-3,5-dideoxy-3-fluoro-D-erythro-α-L-manno-non-2-ulopyranosonate-(2→6)-β-D-galactopyranosyl-(1→4)-2-acetamido-2-deoxy-β-D-glucopyranosyl-(1→2)-α-D-mannopyranosyl-(1→6)]-[β-D-mannopyranosyl]-(1→4)-(2-acetamido-1,2-dideoxy-D-glucopyrano)-[2,1-d]-2-methyloxazoline S1. Representative synthesis of glycan oxazoline. A solution of glycan 17 (14.5 mg, 7.05 μmol, 1 equiv.), 2-Chloro-1,3-dimethyl-1H-benzimidazol-3-ium chloride (CDMBI) (12.2 mg, 0.056 mmol, 8 equiv.) and Et₃N (19.9 μL) in water (121.1 μL) was stirred at 4° C. for 1 h. The reaction mixture was subjected to gel filtration chromatography on a Sephadex G-25 column and eluted with 0.05% aq. Et₃N. The fractions containing the glycan oxazoline product were combined and lyophilized to give the desired product S1 as a white powder (12.6 mg, yield 87%). ¹H NMR (600 MHz, D₂O+0.05% Et₃N): δ 6.09 (d, J=7.2 Hz, 1H, oxa-C1-H_{α}), 5.12 (d, J=52.2 Hz, 2H, sia-C3-H), 5.11 (br, 1H, c-C1-H_{α}), 4.95 (br, 1H, c′-C1-H_{α}), 4.74 (br, 1H, b-C1-H_{β}), 4.60-4.57 (m, 2H, dd′-C1-H_{β}), 4.46-4.45 (m, 2H, ee′-C1-H_{β}), 4.39 (br, 1H), 4.22-4.15 (m, 5H), 3.98-3.49 (m, 55H), 3.43-3.40 (m, 1H), 2.07-2.02 (m, 15H, -5CH₃); ¹³C NMR (150 MHz, D₂O+0.05% Et₃N): δ 174.9, 174.7, 174.7, 170.7, 170.7, 168.5, 103.1, 103.1, 101.4, 99.9, 99.5, 99.4, 99.2, 99.1, 99.0, 96.5, 91.4, 90.2, 80.4, 79.4, 77.9, 76.4, 76.0, 74.6, 74.2, 73.4, 73.4, 72.8, 72.4, 72.3, 72.0, 71.9, 71.5, 70.8, 70.7, 70.2, 69.6, 69.5, 69.4, 69.1, 68.5, 68.2, 67.2, 65.8, 65.7, 65.1, 64.1, 62.6, 61.6, 61.6, 60.1, 54.9, 46.7, 22.4, 22.0, 12.9; ¹⁹F NMR (470 MHz, D₂O+0.05% Et₃N): δ −217.3 (d, JFH=51.3 Hz), −217.3 (d, JFH=51.3 Hz); HRMS (ESI-TOF) m/e: Calcd for C₇₆H₁₂₀F₂N₅O₅₆ [M−H]⁻: 2036.6658 found 2036.6672.

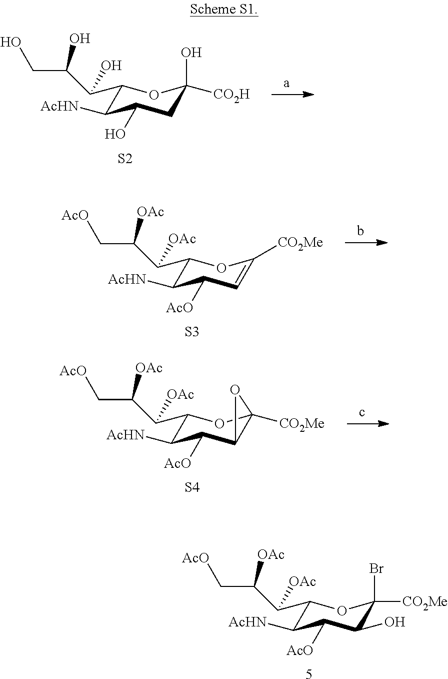

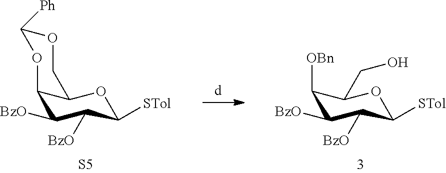

Reagents and conditions in Scheme S1: (a) TFA, MeOH, 60° C., 16 h, then AcCl, r.t., 2 d, then NazHPO₄, CH₃CN, reflux, 20 h, 92%. (b) ref. 3, 80% (three steps). (c) TiBr₄, dichloroethane, r.t., 10 min., 92%. (d) BH₃, Cu(OTf)₂, THF, r.t., 8 h, 96%.

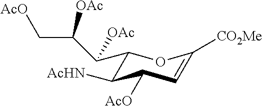

Methyl 5-acetamido-4,7,8,9-tetra-O-acetyl-3,5-dideoxy-2,6-anhydro-D-glycero-D-galacto-non-2-enonate S3.¹⁹ To a stirred solution of compound S2 (20 g, 64.7 mmol, 1 equiv.) in MeOH (600 mL) was added TfOH (4.95 mL, 64.7 mmol, 1 equiv.). The reaction mixture was allowed to stir at 60° C. for 16 h. The solvent was removed by rotary evaporation under reduced pressure and co-evaporated with toluene twice to remove traces of water. The obtained residue was dissolved in AcCl (200 mL) at 0° C. in a round bottom flask, sealed and stirred to room temperature for 2 days. Upon removal of solvent, the obtained residue was diluted with anhydrous acetonitrile (200 mL) and then Na₂HPO₄ (19.6 g, 155 mmol, 2.4 equiv.) was added under argon. The reaction mixture was vigorously stirred at 90° C. until TLC indicated the disappearance of starting material (16 hrs). The solution was filtered through a pad of Celite, dried over MgSO₄, and concentrated in vacuo. The obtained residue was purified by silica gel column chromatography using EtOAc/hexane (4:1) as eluent to give compound S3 as a brown foam (28.2 g, 92%). Spectroscopic data was agreed with reported in the literature previously.¹⁹ R_{f}=0.30 (silica gel, EtOAc); ¹H NMR (600 MHz, CDCl₃): δ 5.95 (d, J=3.0 Hz, 1H), 5.81 (d, J=9.0 Hz, 1H), 5.47-5.44 (m, 2H), 5.32-5.30 (m, 1H), 4.59 (dd, J=12.0, 3.0 Hz, 1H), 4.38-4.33 (m, 2H), 4.15 (d, J=12.0, 7.2 Hz, 1H), 3.76 (s, 3H, —CH₃), 2.08 (s, 3H, —CH₃), 2.03 (s, 3H, —CH₃), 2.02 (s, 3H, —CH₃), 2.01 (s, 3H, —CH₃), 1.88 (s, 3H, —CH₃); ¹³C NMR (150 MHz, CDCl₃): δ 170.7, 170.5, 170.1, 170.1, 170.0, 161.6, 145.0, 107.9, 70.6, 67.8, 67.6, 61.9, 52.5, 46.5, 23.1, 20.8, 20.7, 20.7; HRMS (ESI-TOF) m/e: Calcd for C₂₀H₂₇NO₁₂Na [M+Na]⁺: 496.1425 found 496.1435.

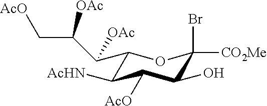

Methyl 5-acetamido-4,7,8,9-tetra-O-acetyl-2-bromo-2,5-dideoxy-D-erythro-α-L-gluco-non-2-ulopyranosonate 3.²⁰ The synthesis of the epoxide S4 was performed from sialic acid using reported procedures.²¹ To a stirred solution of epoxide compound S4 (1.10 g, 2.25 mmol, 1 equiv.) in anhydrous 1,2-dichloroethane (18 mL), was added TiBr₄ (0.91 g, 2.47 mmol, 1.1 equiv.) under argon for 10 min. The solvent was removed by rotary evaporation under high vacuum. The obtained residue was diluted with EtOAc (30 mL), washed with saturated aq. Na₂SO₄ (10 mL), 5% aq. NaHCO₃ (10 mL), and then brine (5 mL). The separated organic layer was dried over MgSO₄, and concentrated in vacuo and the obtained residue was purified by silica gel column chromatography using acetone/hexane (2:1) as eluent to give compound 3 as a white powder (1.18 g, 92%). Spectroscopic data and protocol were identical to that reported previously.²⁰ R_{f}=0.23 (silica gel, acetone: toluene=1:1); ¹H NMR (600 MHz, CDCl₃): δ 5.99 (d, J=7.2 Hz, 1H), 5.42 (dd, J=7.2, 1.2 Hz, 1H), 5.21-5.18 (m, 1H), 5.10 (ddd, J=6.0, 6.0, 2.4 Hz, 1H), 4.38 (dd, J=12.6, 2.4 Hz, 1H), 4.32-4.30 (m, 2H), 3.99 (dd, J=12.6, 6.0 Hz, 1H), 3.86 (s, 3H, —CH₃), 3.78 (d, J=9.0 Hz, 1H), 3.66 (br, 1H, —OH), 2.07 (s, 3H, —CH₃), 2.06 (s, 3H, —CH₃), 2.05 (s, 3H, —CH₃), 2.01 (s, 3H, —CH₃), 1.84 (s, 3H, —CH₃); ¹³C NMR (150 MHz, CDCl₃): δ 171.4, 170.6, 170.3, 169.8, 169.7, 167.0, 98.0, 75.0, 72.8, 72.2, 69.9, 66.5, 61.9, 54.0, 47.2, 22.9, 21.0, 20.7, 20.6; HRMS (ESI-TOF) m/e: Calcd for C₂₀H₂₉BrNO₁₃ [M+H]⁺: 570.0817 found 570.0822.

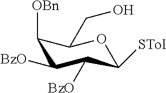

p-Tolyl 2,3-di-O-benzoyl-4-O-benzyl-1-thio-β-D-galactopyranoside 5.²² To a stirred solution of starting material S523 (4.00 g, 6.86 mmol, 1 equiv.) in BH₃-THF complex (1 M in THF, 34.3 mL, 34.3 mmol, 5 equiv.) was added Cu (OTf) 2 (124 mg, 0.34 mmol, 0.05 equiv.) and the reaction mixture was stirred at room temperature for 8 h. Upon completion, the reaction was carefully neutralized with TEA (0.96 mL, 6.86 mmol, 1 equiv.) at 0° C., then diluted with MeOH (15 mL). After removal of solvent in vacuo, the obtained residue was purified by silica gel column chromatography using EtOAc/hexane (1:2) as an eluent to give compound 5 as a white powder (3.85 g, 96%). Spectroscopic data agreed with those reported in the literature previously.²² R_{f}=0.45 (silica gel, EtOAc:hexane=1:1); ¹H NMR (600 MHz, CDCl₃): δ 7.97-7.94 (m, 4H, Ar—H), 7.51-7.48 (m, 2H, Ar—H), 7.38-7.34 (m, 6H, Ar—H), 7.27-7.21 (m, 5H, Ar—H), 5.85 (dd, J=10.2, 10.2 Hz, 1H), 5.36 (dd, J=9.6, 3.0 Hz, 1H), 4.87 (d, J=9.6 Hz, 1H, C1-H_{β}), 4.74 (d, J=12.0 Hz, 1H), 4.47 (d, J=12.0 Hz, 1H), 4.16 (d, J=2.4 Hz, 1H), 3.91 (d, J=11.4, 7.2 Hz, 1H), 3.76 (dd, J=6.0, 6.0 Hz, 1H), 3.61 (dd, J=11.4, 5.4 Hz, 1H), 2.30 (s, 3H, —CH₃), 1.83 (br, 1H); ¹³C NMR (150 MHz, CDCl₃): δ 165.9, 165.2, 138.1, 137.4, 133.4, 133.1, 133.0, 129.8, 129.7, 129.6, 129.5, 128.9, 128.7, 128.5, 128.4, 128.3, 128.1, 127.9, 86.8, 79.0, 76.0, 74.6, 73.7, 68.5, 61.9, 21.1; HRMS (ESI-TOF) m/e: Calcd for C₃₄H₃₂O₇SNa [M+Na]⁺: 607.1761 found 607.1770.

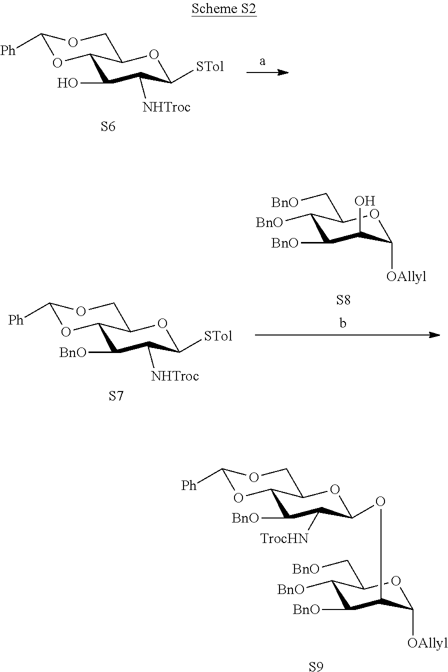

Reagents and conditions in Scheme S2: (a) TESOTf, BnCHO, TESH, toluene, THF, 2 h, −20° C., 90%. (b) NIS, TfOH, 4 Å MS, −40° C., 1.5 h, CH₂Cl₂, 75%. (c) NaBH₃CN, HCl/ether, AW-300, THF, 0° C. to r.t., 16 h, 85%.

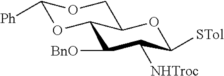

p-Tolyl 3-O-benzyl-4,6-O-benzylidene-2-deoxy-1-thio-2-(2,2,2-trichloroethoxy) carbamoylamino-β-D-glucopyranoside S7. To a stirred solution of starting material S6²⁴ (5.17 g, 9.42 mmol, 1 equiv.) in a mixture of THF/toluene (45 mL, 1:2=v v) was added TESOTf (4.26 mL, 18.8 mmol, 2 equiv.) at −20° C. under argon. After stirring for 45 min at −20° C., benzaldehyde (4.79 mL, 47.1 mmol, 5 equiv.) and triethylsilane (2.26 mL, 14.1 mmol, 1.5 equiv.) were added dropwise to the mixture with stirring. After 2 h at −20° C., TLC indicated the disappearance of starting material and the reaction was quenched with satd. aq. Na₂CO₃, diluted with EtOAc (100 mL) and washed with satd. aq. Na₂CO₃ (30 mL), and brine. The combined organic layers were dried over MgSO₄, filtered and concentrated in vacuo. The resulting residue was purified by silica gel column chromatography first using CH₂Cl₂/toluene (1:4) then EtOAc/toluene (1:10) as eluent to give compound S7 as a white solid (5.40 g, 90%). R_{f}=0.50 (silica gel, EtOAc:toluene=1:25); ¹H NMR (600 MHz, Acetone-d6): δ 7.52-7.50 (m, 2H, Ar—H), 7.42-7.36 (m, 4H, Ar—H), 7.32-7.31 (m, 2H, Ar—H), 7.29-7.22 (m, 4H, Ar—H), 7.18-7.15 (m, 2H, Ar—H), 5.72 (s, 1H, Ph-CH), 5.03 (d, J=10.8 Hz, 1H, C1-H_{β}), 4.90-4.82 (m, 3H), 4.74 (d, J=12.0 Hz, 1H), 4.30 (dd, J=10.2, 5.4 Hz, 1H), 3.95 (dd, J=9.6, 9.0 Hz, 1H), 3.84-3.71 (m, 3H), 3.56 (ddd, J=9.6, 9.6, 4.8 Hz, 1H), 2.83 (s, 2H), 2.32 (s, 3H, —CH₃); ¹³C NMR (150 MHz, Acetone-d6): δ 155.3, 139.8, 139.4, 138.6, 133.3, 130.7, 130.5, 129.8, 129.6, 129.1, 129.0, 129.0, 128.5, 128.2, 127.1, 101.8, 97.1, 88.6, 82.7, 80.8, 75.0, 75.0, 71.2, 69.1, 57.4, 21.1; HRMS (ESI-TOF) m/e: Calcd for C30H30Cl₃NO6SNa [M+Na]⁺: 660.0752 found 660.0749.

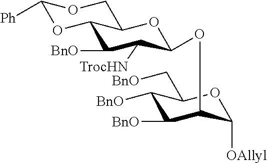

Allyl [3-O-benzyl-4,6-O-benzylidene-2-deoxy-2-(2,2,2-trichloroethoxy) carbonylamino-β-D-glucopyranosyl]-(1→2)-3,4,6-tri-O-benzyl-α-D-mannopyranoside S9. A mixture of acceptor S825 (523 mg, 0.94 mmol, 1 equiv.), donor S7 (900 mg, 1.41mmol, 1.5 equiv.) and activated pulverized 4 Å MS (2.00 g) in anhydrous CH₂Cl₂ (20 mL) was stirred under argon for 1 h. Then, the reaction mixture was cooled to −40° C. and treated with NIS (423 mg, 1.88 mmol, 2 equiv.) and TfOH (0.5 M in Et_{20, 0.47} mL, 0.23 mmol, 0.25 equiv.). After stirring at that temperature for 1.5 h, the TLC analysis indicated disappearance of starting materials and the reaction mixture was quenched with Et₃N (0.2mL), and filtered through a pad of Celite. The filtrate was diluted with CH₂Cl₂ (20 mL), washed with 20% aq. Na2S203 (5 mL), satd. aq. NaHCO3 (10 mL) and brine (5 mL). The separated organic layer was dried over MgSO4 and concentrated in vacuo. The obtained residue was purified by silica gel column chromatography using EtOAc/toluene (1:10) as eluent to give compound S9 as a white foam (710 mg, 75%). R_{f}=0.36 (silica gel, EtOAc: toluene=1:10); ¹H NMR (600 MHz, CDCl₃): δ 7.50-7.48 (m, 2H, Ar—H), 7.39-7.36(m, 6H, Ar—H), 7.33-7.25 (m, 15H, Ar—H), 7.23-7.22 (m, 2H, Ar—H), 5.84 (ddd, J=16.2. 10.8, 5.4 Hz, 1H), 5.55 (s, 1H, Ph-CH), 5.26 (d, J=1.2 Hz, 1H), 5.23-5.20 (m, 1H), 5.16 (dd, J=10.8, 1.2 Hz, 1H), 5.01 (d, J=7.8 Hz, 1H, C1-H_{β}), 4.90 (d, J=10.8 Hz, 1H), 4.83 (d, J=11.4 Hz, 1H), 4.75-4.62 (m, 6H, C1-H_{α}), 4.55-4.51 (m, 3H), 4.33-4.29 (m, 2H), 4.13-4.10 (m, 1H), 4.08 (dd, J=2.4, 2.4 Hz, 1H), 4.00-3.97 (m, 1H), 3.94 (dd, J=9.6, 3.0 Hz, 1H), 3.89 (dd, J=13.2, 6.0 Hz, 1H), 3.82-3.78 (m, 2H), 3.72-3.66 (m, 3H), 3.50-3.46 (m, 1H), 3.12 (d, J=6.0 Hz, 1H); ¹³C NMR (150 MHz, CDCl₃): δ 153.8, 138.6, 138.5, 138.3, 138.1, 137.3, 133.7, 129.0, 128.4, 128.3, 128.2, 128.1, 128.0, 127.8, 127.6, 127.6, 127.4, 126.0, 117.2, 101.2, 98.9, 96.9, 95.5, 82.4, 78.5, 75.6, 75.2, 74.6, 74.4, 74.2, 73.3, 72.2, 72.2, 69.2, 68.6, 68.0, 66.1, 58.1; HRMS (ESI-TOF) m/e: Calcd for C₅₃H₅₆Cl₃NO₁₂Na [M+Na]⁺: 1026.2760 found 1026.2780.

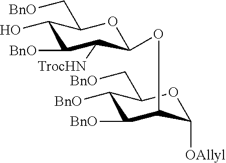

Allyl [3,6-di-O)-benzyl-2-deoxy-2-(2,2,2-trichloroethoxy)carbonylamino-β-D-glucopyranosyl]-(1→2)-3,4,6-tri-O-benzyl-α-D-mannopyranoside 8.²⁷ To a stirred solution of starting material S9 (500 mg, 0.50 mmol, 1 equiv.) in anhydrous THF (12 mL) was added activated pulverized AW 300 MS (1.20 g) under argon. Then, the reaction mixture was cooled to 0° C. and NaCNBH₃ (313 mg, 4.97 mmol, 10 equiv.) was added, followed by a slow addition of HCl·Et₂O (2 M in Et₂O, 2.24 mL, 4.48 mmol, 9 equiv.), and the mixture was continued to stir until TLC indicated the disappearance of starting materials (16 hrs). Upon completion, the reaction mixture was quenched with satd. aq. NaHCO₃ (0.5 mL) and filtered through a pad of Celite. The filtrate was diluted with CH₂Cl₂ (20 mL) then washed with satd. aq. NaHCO₃ (8 mL) and brine (5 mL). The separated organic layer was dried over MgSO₄ and concentrated. The obtained residue was purified by silica gel column chromatography using EtOAc/toluene (1:5) as an eluent to give compound 8 as a white foam (425 mg, 85%). The spectroscopic data were consistent with those reported in the literature.²⁷ R_{f}=0.17 (silica gel, EtOAc:toluene=1:10); ¹H NMR (600 MHz, CDCl₃): δ 7.36-7.23 (m, 23H, Ar—H), 7.20-7.19 (m, 2H, Ar—H), 5.84 (ddd, J=16.2. 10.2, 6.0 Hz, 1H), 5.35 (br, 1H), 5.21 (dd, J=16.2, 1.2 Hz, 1H), 5.14 (dd, J=10.2, 1.2 Hz, 1H), 4.95 (d, J=7.2 Hz, 1H, C1-H_{β}), 4.88 (d, J=10.8 Hz, 1H), 4.78 (d, J=1.8 Hz, 1H, C1-H_{α}), 4.75 (d, J=11.4 Hz, 1H), 4.70-4.63 (m, 5H), 4.60 (d, J=11.4 Hz, 1H), 4.55-4.49 (m, 4H), 4.14-4.07 (m, 3H), 3.96-3.87 (m, 3H), 3.77 (dd, J=10.8, 4.2 Hz, 1H), 3.73-3.70 (m, 3H), 3.66 (dd, J=10.8, 1.8 Hz, 1H), 3.61 (dd, J=9.6, 8.4 Hz, 1H), 3.54-3.51 (m, 1H), 3.08 (m, 1H), 2.71 (m, 1H, —OH); ¹³C NMR (150 MHz, CDCl₃): δ 154.0, 138.5, 138.5, 138.4, 138.1, 137.6, 133.7, 128.5, 128.5, 128.3, 128.3, 128.3, 128.2, 128.0, 128.0, 127.8, 127.7, 127.7, 127.6, 127.6, 127.5, 117.3, 79.2, 78.2, 75.1, 74.6, 74.3, 74.2, 73.8, 73.7, 73.4, 73.3, 72.0, 71.7, 70.9, 69.3, 68.0, 57.5; HRMS (ESI-TOF) m/e: Calcd for C₅₃H₅₈Cl₃NO₁₂Na [M+Na]⁺: 1028.2917 found 1028.2927.

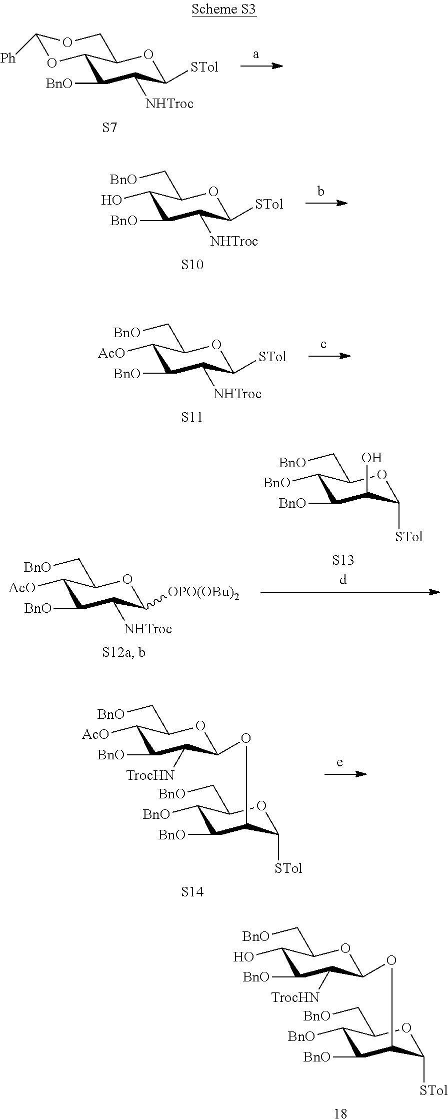

Reagents and conditions in Scheme S3: (a) NaBH₃CN, HCl/ether, AW-300, THF, 0° C. to r.t., 16 h, 90%. (b) AczO, pyridine, r.t., 16 h, 98%. (c) NIS, TfOH, HOPO (OBu) 2, 4 Å MS, −30° C., 2 h, CH₂Cl₂, 90%. (d) TMSOTf, 4 Å MS, −50° C., 1 h, CH₂Cl₂, 87%. (e) NaOMe, MeOH/CH₂Cl₂, 0° C. to r.t., 76%.

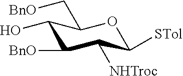

p-Tolyl 3,6-di-O-benzyl-2-deoxy-1-thio-2-(2,2,2-trichloroethoxy)carbamoylamino-β-D-glucopyranoside S10.²⁸ To a stirred solution of starting material S7 (1.00 g, 1.56 mmol, 1 equiv.) in anhydrous THF (30 mL) was added activated pulverized AW 300 MS (3.00 g) under argon. Then, the reaction mixture was cooled to 0° C. and NaCNBH₃ (0.98 g, 15.6 mmol, 10 equiv.) was added, followed by a slow addition of HCl ·Et₂O (2 M in Et₂O, 7.04 mL, 14.1 mmol, 9 equiv.), and the mixture was stirred until TLC indicated the disappearance of starting materials (16 h). Upon completion, the reaction mixture was quenched with satd. aq. NaHCO₃ (0.5 mL) and filtered through a pad of Celite. The filtrate was diluted with CH₂Cl₂ (50 mL) then washed with satd. aq. NaHCO₃ (15 mL) and brine (10 mL). The separated organic layer was dried over MgSO₄ and concentrated. The obtained residue was purified by silica gel column chromatography using EtOAc/hexane/CH₂Cl₂ (1:3:1) as eluent to give compound S10 as a white foam (902 mg, 90%). The spectroscopic data were consistent with those reported in the literature.²⁸ R_{f}=0.29 (silica gel, EtOAc:hexane=1:3); ¹H NMR (600 MHz, CDCl₃): δ 7.40-7.38 (m, 2H, Ar—H), 7.36-7.27 (m, 10H, Ar—H), 7.04-7.03 (m, 2H, Ar—H), 5.20 (d, J=7.8 Hz, 1H), 4.84 (d, J=10.2 Hz, 1H, C1-H_{β}), 4.77-4.72 (m, 4H), 4.58-4.52 (m, 2H), 3.76 (d, J=4.8 Hz, 2H), 3.71-3.64 (m, 2H), 3.50-3.49 (m, 1H), 3.42-3.38 (m, 1H), 2.90 (br, 1H, —OH), 2.29 (s, 3H, —CH₃); ¹³C NMR (150 MHz, CDCl₃): δ 153.8, 138.1, 138.1, 137.7, 133.0, 129.6, 128.6, 128.5, 128.4, 128.1, 127.9, 127.8, 127.7, 95.5, 86.1, 81.9, 78.0, 74.5, 74.4, 73.6, 72.5, 70.4, 55.9, 21.1; HRMS (ESI-TOF) m/e: Calcd for C₃₀H₃₂Cl₃NO₆SNa [M+Na]⁺: 662.0908 found 662.0919.

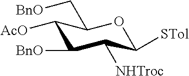

p-Tolyl 4-O-acetyl-3,6-di-O-benzyl-2-deoxy-1-thio-2-(2,2,2-trichloroethoxy)carbamoylamino-β-D-glucopyranoside S11. To a stirring solution of starting material S10 (0.97 g, 1.51 mmol, 1 equiv.) in pyridine (12 mL) was added Ac2O (6 mL). The reaction mixture was vigorously stirred for 16 h at room temperature, then concentrated in vacuo and purified by silica gel column chromatography using EtOAc/hexane/CH₂Cl₂ (1:4:1) as eluent to give compound S11 as a white powder (1.01 g, 98%). R_{f}=0.47 (silica gel, EtOAc:hexane=1:3); ¹H NMR (600 MHz, CDCl₃): δ 7.41-7.40 (m, 2H, Ar—H), 7.34-7.25 (m, 8H, Ar—H), 7.22-7.21 (m, 2H, Ar—H), 7.03-7.01 (m, 2H, Ar—H), 5.29 (d, J=7.2 Hz, 1H), 5.04 (d, J=10.2 Hz, 1H, C1-H_{β}), 4.98 (dd, J=9.6, 9.6 Hz, 1H), 4.78 (d, J=12.0 Hz, 1H), 4.70 (d, J=12.0 Hz, 1H), 4.62 (d, J=11.4 Hz, 1H), 4.57 (d, J=11.4 Hz, 1H), 4.50 (br, 2H), 4.05 (dd, J=9.6, 9.0 Hz, 1H), 3.65-3.63 (m, 1H), 3.59-3.55 (m, 2H), 3.33 (ddd, J=9.6, 9.6, 9.6 Hz, 1H), 2.29 (s, 3H, —CH₃), 1.87 (s, 3H, —CH₃); ¹³C NMR (150 MHz, CDCl₃): δ 169.8, 153.7, 138.4, 138.0, 137.7, 133.3, 129.8, 128.6, 128.5, 128.4, 128.2, 128.1, 128.1, 127.9, 127.9, 127.7, 95.5, 85.3, 79.4, 77.7, 74.4, 73.6, 71.4, 69.7, 56.5, 21.1, 20.9; HRMS (ESI-TOF) m/e: Calcd for C₃₂H₃₄Cl₃NO₇SNa [M+Na]⁺: 704.1014 found 104.1031.

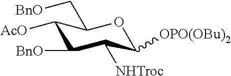

Dibutyl 4-O-acetyl-3,6-di-O-benzyl-2-deoxy-1-thio-2-(2,2,2-trichloroethoxy)carbamoylamino-α or β-D-glucopyranoside phosphate S12a, b. A mixture of compound S11 (990 mg, 1.45 mmol, 1 eq.), dibutyl phosphate (1.15 mL, 5.80 mmol, 3 eq.) and activated pulverized 4 Å MS (1.80 g) in anhydrous CH₂Cl₂ (18 mL) was stirred under argon for 1 h. Then it was cooled to −30° C. with stirring, and NIS (978 mg, 4.35 mmol, 2 eq.) was added followed by TfOH (0.5 M in Et₂O, 0.87 mL, 0.43 mmol, 0.3 eq.). After 2 h, the TLC analysis indicated disappearance of starting materials and the reaction mixture was quenched by satd. aq. NaHCO₃ (0.5 mL) and filtered through a pad of Celite. The filtrate was diluted with CH₂Cl₂ (50 mL), washed with 20% aq. Na₂S₂O₃ (10 mL), satd. aq. NaHCO₃ (5 mL) and brine (5 mL). The separated organic layer was dried over MgSO₄ and concentrated in vacuo. The obtained residue was purified by silica gel column chromatography using EtOAc/toluene (1:3) as eluent to give compound S12 as a white foam (1.00 g, 90%); anomeric mixture (α:β=1:3). β-anomer S12b: R_{f}=0.32 (silica gel, EtOAc:toluene=1:3); ¹H NMR (600 MHz, CDCl₃): δ 7.30-7.22 (m, 10H, Ar—H), 5.81 (br, 1H), 5.35 (dd, J=7.8, 7.8 Hz, 1H, C1-H_{β}), 5.09 (dd, J=9.6, 9.0 Hz, 1H), 4.74 (d, J=12.0 Hz, 1H), 4.65-4.58 (m, 3H), 4.49-4.44 (m, 2H), 4.06-3.97 (m, 4H), 3.88 (dd, J=9.6, 9.0 Hz, 1H), 3.77-3.74 (m, 1H), 3.69 (ddd, J=9.6, 4.2, 4.2 Hz, 1H), 3.53-3.52 (m, 2H), 1.83 (s, 3H, —CH₃), 1.61-1.54 (m, 4H), 1.36-1.31 (m, 4H), 0.93-0.85 (m, 6H); ¹³C NMR (150 MHz, CDCl₃): δ 169.5, 154.2, 137.6, 137.6, 128.5, 128.4, 128.3, 127.9, 127.9, 127.7, 127.7, 96.5, 95.3, 78.3, 74.5, 73.9, 73.6, 73.5, 70.6, 69.2, 68.1, 68.1, 68.1, 68.0, 57.2, 57.2, 32.1, 32.0, 32.0, 20.7, 18.6, 18.6, 13.5; HRMS (ESI-TOF) m/e: Calcd for C₃₃H₄₅Cl₃NO₁₁PNa [M+Na]⁺: 790.1688 found 790.1685. α-anomer S12a: R_{f}=0.44 (silica gel, EtOAc:toluene=1:3); ¹H NMR (600 MHz, CDCl₃): δ 7.31-7.22 (m, 10H, Ar—H), 5.67 (dd, J=6.0, 3.6 Hz, 1H, C1-H_{α}), 5.21-5.16 (m, 2H), 4.75 (d, J=12.0 Hz, 1H), 4.62-4.56 (m, 3H), 4.49-4.44 (m, 2H), 4.11-3.99 (m, 6H), 3.77 (dd, J=10.2, 9.6 Hz, 1H), 3.50-3.49 (m, 2H), 1.89 (s, 3H, —CH₃), 1.60-1.55 (m, 4H), 1.35-1.31 (m, 4H), 0.92-0.86 (m, 6H); ¹³C NMR (150 MHz, CDCl₃): δ 169.3, 154.0, 137.4, 137.4, 128.4, 128.2, 127.8, 127.8, 127.8, 127.6, 127.6, 127.6, 96.4, 96.3, 95.2, 76.4, 74.6, 73.6, 73.5, 71.1, 70.1, 68.6, 68.1, 68.0, 68.0, 67.9, 54.4, 54.3, 32.1, 32.0, 20.7, 18.5, 18.5, 13.5; HRMS (ESI-TOF) m/e: Calcd for C₃₃H₄₅Cl₃NO₁₁PNa [M+Na]⁺: 790.1688 found 790.1688.

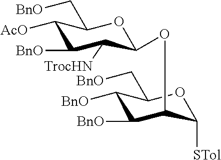

p-Tolyl [4-O-acetyl-3,6-di-O-benzyl-2-deoxy-2-(2,2,2-trichloroethoxy)carbonylamino-β-D-glucopyranosyl]-(1→2)-3,4,6-tri-O-benzyl-1-thio-α-D-mannopyranoside S14. A mixture of acceptor S1329 (407 mg, 0.73 mmol, 1 equiv.) and donor S12 (900 mg, 1.17 mmol, 1.6 equiv.) and activated pulverized 4 Å molecular sieves (200 mg) in anhydrous CH₂Cl₂ (2 mL) was stirred under argon for 30 min. Then it was cooled to −50° C. followed by addition of TMSOTf (0.21 mL, 1.17 μmol, 1.6 equiv. with respect to acceptor) with stirring until TLC analysis indicated disappearance of starting materials (1 h). The reaction mixture was quenched with Et₃N (0.30 mL), diluted with CH₂Cl₂ (20 mL), and filtered through a pad of Celite. The filtrate was washed twice with satd aq. NaHCO3 (8 mL) and brine (4 mL). The organic phase was dried over MgSO₄, filtered, and concentrated in vacuo. The obtained residue was purified by silica gel column chromatography using EtOAc/hexane (1:3) as eluent to give compound S14 as a white powder (710 mg, 87%). R_{f}=0.26 (silica gel, EtOAc:hexane=1:3); ¹H NMR (600 MHz, CDCl₃): δ 7.37-7.36 (m, 2H, Ar—H), 7.33-7.14 (m, 25H, Ar—H), 7.05-7.03 (m, 2H, Ar—H), 5.33 (d, J=1.8 Hz, 1H, C1-H_{α}), 5.26 (d, J=4.8 Hz, 1H), 5.07 (d, J=9.0 Hz, 1H, C1-H_{β}), 4.93-4.90 (m, 2H), 4.77 (d, J=11.4 Hz, 1H), 4.60-4.52 (m, 5H), 4.46-4.34 (m, 5H), 4.27 (dd, J=8.4, 8.4 Hz, 1H), 4.19 (dd, J=9.6, 2.4 Hz, 1H), 4.15 (d, J=12.0 Hz, 1H), 4.07 (dd, J=9.6, 9.0 Hz, 1H), 3.84-3.81 (m, 2H), 3.64-3.63 (m, 2H), 3.57 (dd, J=10.8, 6.0 Hz, 1H), 3.51 (dd, J=11.4, 3.0 Hz, 1H), 3.01 (dd, J=6.6 Hz, 1H), 2.28 (s, 3H, —CH₃), 1.82 (s, 3H, —CH₃); ¹³C NMR (150 MHz, CDCl₃): δ 169.7, 154.2, 138.5, 138.3, 138.0, 137.8, 137.7, 137.6, 132.5, 130.3, 129.8, 129.7, 129.0, 128.4, 128.4, 128.3, 128.2, 128.2, 128.2, 128.2, 127.9, 127.8, 127.8, 127.7, 127.7, 127.6, 127.6, 127.6, 127.4, 125.2, 97.1, 95.4, 86.2, 78.3, 75.2, 75.1, 74.5, 74.1, 74.0, 73.6, 73.4, 73.1, 72.5, 71.7, 71.3, 70.0, 69.2, 58.1, 21.1, 20.8; HRMS (ESI-TOF) m/e: Calcd for C₅₉H₆₂Cl₃NO₁₂SNa [M+Na]⁺: 1136.2950 found 1136.2978.

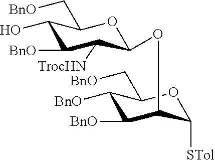

p-Tolyl [3,6-di-O-benzyl-2-deoxy-2-(2,2,2-trichloroethoxy)carbonylamino-β-D-glucopyranosyl]-(1→2)-3,4,6-tri-O-benzyl-1-thio-α-D-mannopyranoside 18. To a well-stirred solution of compound S14 (203 mg, 0.18 mmol, 1 equiv.) in a mixture of CH₂Cl₂/MeOH (10 mL, 1:1=v/v) was added NaOMe (2.95 mg, 0.055 mmol, 0.3 equiv.) at 0° C. After 20 min, the ice bath was removed and the reaction mixture was warmed up to room temperature with stirring until TLC analysis indicated the disappearance of starting materials (4 hrs). Upon completion, the reaction mixture was neutralized with IR-120, filtered, and concentrated in vacuo. The obtained residue was purified by silica gel column chromatography using EtOAc/hexane (1:2) as eluent to give compound 18 as a white foam (149 mg, 76%). R_{f}=0.14 (silica gel, EtOAc:hexane=1:3); ¹H NMR (600 MHz, CDCl₃): δ 7.39-7.37 (m, 2H, Ar—H), 7.34-7.24 (m, 25H, Ar—H), 7.06-7.05 (m, 2H, Ar—H), 5.33 (d, J=1.8 Hz, 1H, C1-H_{α}), 5.24 (d, J=4.8 Hz, 1H), 5.00 (d, J=7.8 Hz, 1H, C1-H_{β}), 4.93 (d, J=10.8 Hz, 1H), 4.78 (d, J=11.4 Hz, 1H), 4.69-4.64 (m, 2H), 4.61 (d, J=11.4 Hz, 2H), 4.57-4.51 (m, 4H), 4.43 (d, J=11.4 Hz, 1H), 4.34 (dd, J=2.4, 2.4 Hz, 1H), 4.22-4.20 (m, 2H), 4.07-4.04 (m, 2H), 3.84-3.82 (m, 2H), 3.77-3.72 (m, 2H), 3.67-3.65 (m, 1H), 3.59-3.54 (m, 2H), 3.01 (d, J=7.2 Hz, 1H), 2.68 (br, 1H, —OH), 2.30 (s, 3H, —CH₃); ¹³C NMR (150 MHz, CDCl₃): δ 154.2, 138.5, 138.4, 138.3, 137.9, 137.7, 137.6, 132.5, 130.3, 129.8, 128.5, 128.5, 128.4, 128.3, 128.1, 128.0, 127.9, 127.8, 127.8, 127.7, 127.6, 127.6, 127.5, 97.5, 95.4, 86.2, 79.1, 78.4, 75.3, 75.2, 74.6, 74.4, 74.2, 73.8, 73.8, 73.2, 73.1, 72.5, 71.4, 70.9, 69.3, 57.8, 21.1; HRMS (ESI-TOF) m/e: Calcd for C₅₇H₆₀Cl₃NO₁₁SNa [M+Na]⁺: 1094.2845 found 1094.2881.

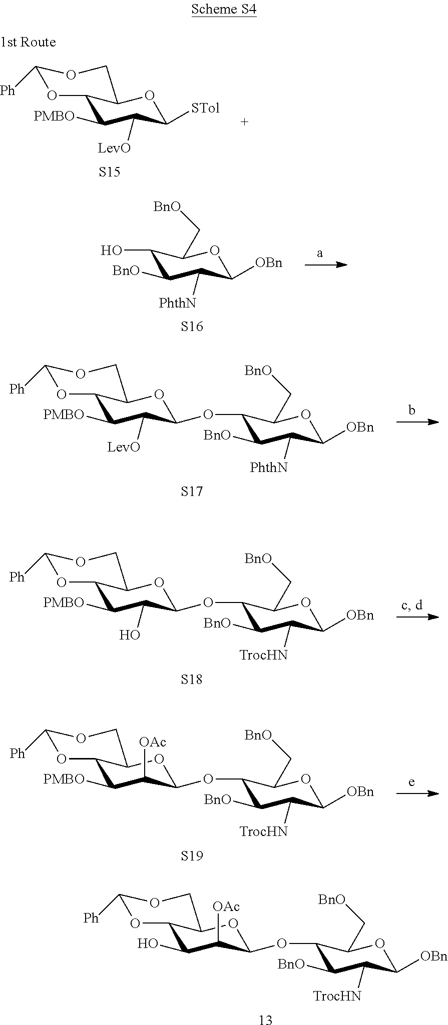

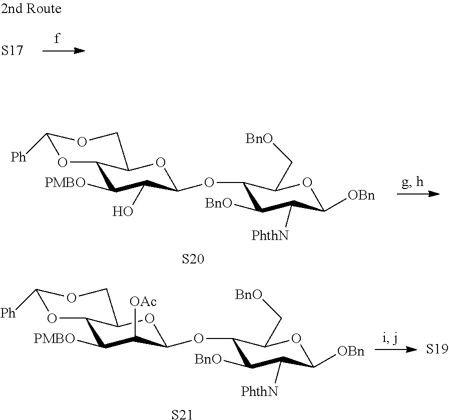

Reagents and conditions in Scheme S4: (a) NIS, TfOH, 4 Å MS, −40° C., 1 h, CH₂Cl₂, 93%. (b) ethylenediamine/BuOH (1:4), 90° C., 2 h, then NaHCO₃, TrocCl, CH₂Cl₂, 0° C., 3 h, 81%. (c) Tf₂O, pyridine, CH₂Cl₂, 0° C., 4 h. (d) Bu₄NOAc, toluene, sonication, r.t., 8 h, then NaHCO₃, TrocCl, CH₂Cl₂, 0° C., 3 h, 61% (2 steps). (e) DDQ, CH₂Cl₂, phosphate buffer (pH=7), 0° C. to r.t., 3 h, 85%. (f) hydrazine acetate, THF, r.t., 16 h, 92%. (g) Tf₂O, pyridine, CH₂Cl₂, 0° C., 2 h. (h) Bu₄NOAc, toluene, sonication, r.t., 8 h, 93% (2 steps). (i) ethylenediamine/BuOH (1:4), 90° C., 2 h, then NaHCO₃, TrocCl, CH₂Cl₂, 0° C., 3 h. (j) Ac₂O, pyridine, CH₂Cl₂, r.t., 16 h, 83% (2 steps).

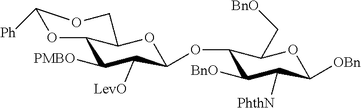

Benzyl [4,6-O-benzylidine-3-O-p-methoxy-benzyl-2-O-levulinoyl-β-D-glucopyranosyl]-(1→4)-3,6-di-O-benzyl-2-deoxy-2-phthalimido-β-D-glucopyranoside S17. A mixture of acceptor S16.³⁰ A mixture of acceptor S1630 (1.59 g, 2.74 mmol, 1 equiv.), donor S1526 (2.11 g, 3.57 mmol, 1.3 equiv.) and activated pulverized 4 Å MS (5.00 g) in anhydrous CH₂Cl₂ (50 mL) was stirred under argon for 1 h. Then, the reaction mixture was cooled to −40° C. and NIS (1.23 g, 0.49 mmol, 2 equiv.) was added, followed by TfOH (0.5 M in Et₂O, 1.37 mL, 0.69 mmol, 0.25 equiv.). The reaction was continued until TLC indicated the disappearance of starting materials (2 h). Upon completion, the reaction mixture was quenched with Et₃N (0.7 mL) and filtered through a pad of Celite. The filtrate was diluted with CH₂Cl₂ (50 mL), washed with 20% aq. Na₂S₂O₃ (15 mL), satd. aq. NaHCO₃ (20 mL), and brine (10 mL). The separated organic layer was dried over MgSO₄ and concentrated in vacuo. The obtained residue was purified by silica gel column chromatography using EtOAc/hexane (2:3) as eluent to give compound S17 as a white foam (2.68 g, 93%). R_{f}=0.43 (silica gel, acetone: toluene=1:6); ¹H NMR (600 MHz, CDCl₃): δ 7.75 (br, 1H, Ar—H), 7.63 (br, 2H, Ar—H), 7.50 (br, 1H, Ar—H), 7.46-7.45 (m, 2H, Ar—H), 7.39-7.32 (m, 7H, Ar—H), 7.29-7.26 (m, 1H, Ar—H), 7.20-7.18 (m, 2H, Ar—H), 7.07-7.05 (m, 1H, Ar—H), 7.02-7.01 (m, 4H, Ar—H), 6.98-6.97 (m, 2H, Ar—H), 6.87-6.83 (m, 5H, Ar—H), 5.44 (s, 1H, Ph-CH), 5.10-5.08 (m, 1H, C1-H_{β}), 4.92 (dd, J=9.0, 8.4 Hz, 1H), 4.81-4.71 (m, 4H), 4.58 (d, J=7.8 Hz, 1H, C1-H_{β}), 4.56 (d, J=4.2 Hz, 1H), 4.47 (d, J=6.0 Hz, 1H), 4.45 (d, J=6.6 Hz, 1H), 4.36 (d, J=12.6 Hz, 1H), 4.23 (dd, J=10.2, 4.8 Hz, 1H), 4.20-4.19 (m, 2H), 4.09-4.06 (m, 1H), 3.87 (dd, J=11.4, 3.0 Hz, 1H), 3.79-3.78 (m, 1H), 3.78 (s, 3H, —CH₃), 3.59 (dd, J=9.6, 9.0 Hz, 1H), 3.55-3.51 (m, 2H), 3.43 (dd, J=10.2, 10.2 Hz, 1H), 3.17 (ddd, J=9.6, 9.6, 4.8 Hz, 1H), 2.81-2.76 (m, 1H), 2.70-2.65 (m, 1H), 2.50-2.38 (m, 2H), 2.20 (s, 3H, —CH₃); ¹³C NMR (150 MHz, CDCl₃): δ 206.2, 171.1, 159.2, 138.6, 138.1, 137.3, 133.5, 131.6, 130.4, 129.4, 129.0, 128.5, 128.2, 128.1, 127.9, 127.8, 127.7, 127.5, 127.5, 127.0, 126.0, 123.1, 113.6, 101.1, 100.6, 97.4, 81.7, 78.0, 77.9, 76.6, 74.8, 74.5, 73.7, 73.6, 70.7, 68.6, 67.7, 65.8, 55.7, 55.3, 37.7, 29.9, 27.8; HRMS (ESI-TOF) m/e: Calcd for C₆₁H₆₁NO₁₅Na [M+Na]⁺: 1070.3933 found 1070.3933.

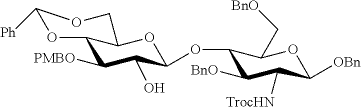

Benzyl [4,6-O-benzylidine-3-O-p-methoxy-benzyl-β-D-glucopyranosyl]-(1→4)-3,6-di-O-benzyl-2-deoxy-2-(2,2,2-trichloroethoxy) carbonylamino-β-D-glucopyranoside S18. A solution of disaccharide S17 (625 mg, 0.596 mmol, 1 equiv.) in ethylene diamine/n-BuOH (7 mL, 2:8=v/v) was stirred at 90° C. for 2 h. The solvent was removed by rotary evaporation under high vacuum and co-evaporated with toluene twice to remove traces of water. The obtained residue was dissolved in anhydrous CH₂Cl₂ (15 mL) and treated with NaHCO₃ (250 mg, 2.98 mmol, 5 equiv.) and 2,2,2-trichloro ethyl chloroformate (0.41 mL, 2.98 mmol, 5 equiv.) at 0° C. under argon for 3 h. Upon completion, the reaction mixture was diluted with CH₂Cl₂ (20 mL), washed with water (20 mL) and brine (10 mL). The organic layer was dried over MgSO₄ and concentrated in vacuo. The obtained residue was purified by silica gel column chromatography using EtOAc/toluene (1:4) as eluent to give compound S18 as a white powder (481 mg, 81%). R_{f}=0.60 (silica gel, EtOAc:toluene=1:2); ¹H NMR (600 MHz, CDCl₃): δ 7.47-7.46 (m, 2H, Ar—H), 7.40-7.25 (m, 20H, Ar—H), 6.86-6.85 (m, 2H, Ar—H), 5.46 (s, 1H, Ph-CH), 5.12 (br, 1H), 4.88-4.85 (m, 3H), 4.71-4.55 (m, 9H, 2C1-H_{β}), 4.06-4.03 (m, 2H), 3.99 (dd, J=11.4, 3.6 Hz, 1H), 3.87 (br, 1H), 3.80 (dd, J=11.4, 1.8 Hz, 1H), 3.78 (s, 3H, —CH₃), 3.56 (dd, J=9.6, 9.0 Hz, 1H), 3.52-3.45 (m, 5H), 3.15 (ddd, J=9.6, 9.6, 4.8 Hz, 1H), 3.03 (br, 1H, —OH); ¹³C NMR (150 MHz, CDCl₃): δ 159.3, 153.8, 138.4, 137.7, 137.3, 137.2, 130.4, 129.6, 128.9, 128.4, 128.4, 128.3, 128.2, 128.0, 127.8, 127.8, 127.6, 127.4, 126.0, 113.8, 103.2, 101.1, 99.2, 95.5, 81.3, 79.9, 79.2, 77.7, 74.9, 74.5, 74.3, 74.2, 73.5, 70.7, 68.6, 68.2, 66.2, 57.6, 55.2; HRMS (ESI-TOF) m/e: Calcd for C₅₁H₅₄C₁₃NO₁₃Na [M+Na]⁺: 1016.2553 found 1016.2554.

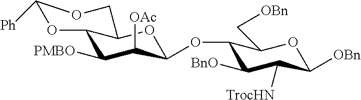

Benzyl [2-O-acetyl-4,6-O-benzylidine-3-O-p-methoxy-benzyl-β-D-mannopyranosyl]-(1→4)-3,6-di-O-benzyl-2-deoxy-2-(2,2,2-trichloroethoxy)carbonylamino-β-D-glucopyranoside S19. From S18: To a stirred solution of disaccharide S18 (1.02 g, 1.02 mmol, 1 equiv.) in anhydrous CH₂Cl₂ (13 mL) was added anhydrous pyridine (0.58 mL, 7.17 mmol, 7 equiv.), followed by dropwise addition of Tf₂O (0.30 mL, 1.79 mmol, 1.75 equiv.) at 0° C. under argon. The reaction mixture was stirred at 0° C. until TLC indicated the disappearance of starting material (4 hrs). Upon completion, it was diluted with CH₂Cl₂ (20 mL), washed with 0.5N HCl (20 mL) and brine (10 mL). The separated organic layer was dried over MgSO₄ and concentrated in vacuo. The crude residue was mixed with Bu₄NOAc (927 mg, 3.07 mmol, 3 equiv.) and dissolved in toluene (20 mL). The solvent was removed in vacuo and the residue was co-evaporated with toluene twice; then, redissolved in anhydrous toluene (13 mL) and the mixture was sonicated for 8 h. During this time, we observed a partial deprotection of NHTroc and the reaction mixture was concentrated under high vacuum. The obtained residue was dissolved in CH₂Cl₂ (13 mL) and treated with NaHCO₃ (430 mg, 5.12 mmol, 5 equiv.) and 2,2,2-trichloro ethyl chloroformate (0.71 mL, 5.12 mmol, 5 equiv.) at 0° C. under argon. After stirring for 3 h, the reaction mixture was diluted with CH₂Cl₂ (20 mL), washed with water (20 mL) and brine (10 mL). The separated organic layer was dried over MgSO₄ and concentrated in vacuo. The obtained residue was purified by silica gel column chromatography using EtOAc/toluene (1:5) as eluent to give compound S19 as a white powder (650 mg, 61%).

From S21: A solution of disaccharide S21 (1.20 g, 1.21 mmol, 1 equiv.) in ethylene diamine/n-BuOH (15 mL, 2:8=v/v) was stirred at 90° C. for 2 h. After removal of solvent, the crude product was co-evaporated with toluene twice. The obtained residue was dissolved in CH₂Cl₂ (30 mL) and treated with NaHCO₃ (508 mg, 6.05 mmol, 5 equiv.) and 2,2,2-trichloro ethyl chloroformate (0.83 mL, 6.05 mmol, 5 equiv.) at 0° C. under argon. After 3 h, the reaction mixture was diluted with CH₂Cl₂ (45 mL), washed with water (30 mL) and brine (15 mL). The organic layer was dried over MgSO₄, concentrated, co-evaporated with toluene twice, and evaporated in vacuo. The obtained residue was subjected directly to the acetylation conditions Ac₂O (7 mL) in pyridine (14 mL), with the temperature slowly warmed up from 0° C. to room temperature in16 h. The reaction mixture was concentrated under high vacuum and purified by silica gel column chromatography using acetone/toluene (1:5) as eluent to give compound S19 as a white powder (1.04 g, 83%). R_{f}=0.54 (silica gel, EtOAc:toluene=1:3); ¹H NMR (600 MHz, CDCl₃): δ 7.48-7.46 (m, 2H, Ar—H), 7.39-7.35 (m, 3H, Ar—H), 7.33-7.25 (m, 14H, Ar—H), 7.24-7.21 (m, 3H, Ar—H), 6.84-6.82 (m, 2H, Ar—H), 5.50 (s, 1H, Ph-CH), 5.41 (d, J=3.0 Hz, 1H), 5.09 (br, 1H), 4.92 (d, J=10.8 Hz, 1H), 4.88 (d, J=12.0 Hz, 1H), 4.72-4.62 (m, 5H, 2C1-H_{β}), 4.58-4.56 (m, 3H), 4.47-4.44 (m, 2H), 4.08 (dd, J=10.2, 4.8 Hz, 1H), 4.04 (dd, J=9.0, 8.4 Hz, 1H), 3.87-3.84 (m, 2H), 3.78-3.72 (m, 5H, —CH₃), 3.59 (dd, J=10.2, 10.2 Hz, 1H), 3.45-3.43 (m, 2H), 3.38 (ddd, J=8.4, 8.4, 8.4 Hz, 1H), 3.10 (ddd, J=9.6, 9.6, 4.8 Hz, 1H), 2.07 (s, 3H, —CH₃); ¹³C NMR (150 MHz, CDCl₃): δ 170.3, 159.3, 153.8, 138.4, 137.8, 137.4, 137.1, 129.7, 129.2, 128.9, 128.5, 128.4, 128.3, 128.2, 128.0, 128.0, 127.9, 127.8, 127.7, 126.1, 113.8, 101.4, 99.1, 99.1, 77.8, 75.3, 74.4, 73.5, 71.4, 70.8, 69.1, 68.5, 68.4, 67.0, 57.5, 55.2, 21.0; HRMS (ESI-TOF) m/e: Calcd for C₅₃H₅₆Cl₃NO₁₄Na [M+Na]⁺: 1058.2658 found 1058.2657.

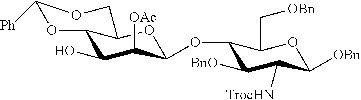

Benzyl [2-O-acetyl-4,6-O-benzylidine-β-D-mannopyranosyl]-(1→4)-3,6-di-O benzyl-2-deoxy-2-(2,2,2-trichloroethoxy)carbonylamino-β-D-glucopyranoside 13. To a stirring solution of S19 (120 mg, 0.12 mmol, 1 equiv.) in a mixture of CH₂Cl₂/phosphate buffer (pH=7) (3 mL, 9:1=v/v) was added 2,3-dichloro-5,6-dicyanobenzoquinone (52.5 mg, 0.23 mmol, 2.2 equiv.) at 0° C. The reaction mixture was vigorously stirred until TLC indicated the disappearance of starting material (3 hrs). Upon completion, the reaction mixture was diluted with CH₂Cl₂ (10 mL), washed with satd. aq. NaHCO₃ (8 mL) and brine (4 mL), and the organic phase was dried over MgSO₄, filtered and concentrated. The obtained residue was purified by silica gel column chromatography using EtOAc/toluene (1:4) as eluent to give compound 13 as a white powder (90 mg, 85%). R_{f}=0.29 (silica gel, EtOAc:toluene=1:3); ¹H NMR (600 MHz, CDCl₃): δ 7.45-7.44 (m, 2H, Ar—H), 7.39-7.34 (m, 7H, Ar—H), 7.33-7.26 (m, 11H, Ar—H), 5.47 (s, 1H, Ph-CH), 5.23 (d, J=3.6 Hz, 1H), 5.06 (br, 1H), 4.91 (d, J=11.4 Hz, 1H), 4.87 (d, J=12.0 Hz, 1H), 4.74 (d, J=12.0 Hz, 1H), 4.68-4.61 (m, 4H, 2C1-H_{β}), 4.58-4.56 (m, 2H), 4.48 (d, J=12.0 Hz, 1H), 4.08 (dd, J=10.2, 4.8 Hz, 1H), 4.04 (dd, J=9.0, 9.0 Hz, 1H), 3.83 (br, 1H), 3.78 (dd, J=11.4, 3.0 Hz, 1H), 3.73-3.70 (m, 2H), 3.64 (dd, J=9.6, 3.6 Hz, 1H), 3.56 (dd, J=10.2, 10.2 Hz, 1H), 3.43-3.36 (m, 2H), 3.11 (ddd, J=9.6, 9.6, 4.8 Hz, 1H), 2.21 (d, J=3.6 Hz, 1H), 2.11 (s, 3H, —CH₃); ¹³C NMR (150 MHz, CDCl₃): δ 170.5, 153.8, 138.5, 137.8, 137.1, 137.0, 129.3, 128.6, 128.6, 128.4, 128.4, 128.3, 128.1, 128.0, 128.0, 127.9, 127.8, 127.7, 126.2, 102.1, 99.1, 99.0, 78.5, 78.2, 74.4, 74.4, 73.6, 71.2, 70.8, 69.7, 68.4, 68.4, 66.7, 57.5; HRMS (ESI-TOF) m/e: Calcd for C₄₅H₄₉C₁₃NO₁₃ [M+H]⁺: 916.2264 found 916.2252.

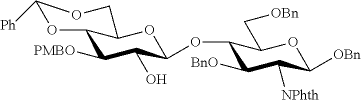

Benzyl [4,6-O-benzylidine-3-O-p-methoxy-benzyl-β-D-glucopyranosyl]-(1→4)-3,6-di-O-benzyl-2-deoxy-2-phthalimido-β-D-glucopyranoside S20. To a stirring solution of starting material S17 (2.48 g, 2.37 mmol, 1 equiv.) in anhydrous THF (55 mL) was added hydrazine acetate (327 mg, 3.55 mmol, 1.5 equiv.) at room temperature under argon. The reaction mixture was vigorously stirred until TLC indicated the disappearance of starting material (16 hrs). Upon completion, the reaction mixture was diluted with EtOAc (150 mL), washed with water (60 mL) and brine (30 mL). The organic phase was dried over MgSO₄, filtered, and concentrated in vacuo. The obtained residue was purified by silica gel column chromatography using EtOAc/Hexane (1:2) as eluent to give compound S20 as a white foam (2.07 g, 92%). R_{f}=0.54 (silica gel, EtOAc:hexane=1:1); ¹H NMR (600 MHz, CDCl₃): δ 7.79-7.54 (m, 4H, Ar—H), 7.51-7.44 (m, 2H, Ar—H), 7.38-7.33 (m, 7H, Ar—H), 7.30-7.27 (m, 3H, Ar—H), 7.08-7.04 (m, 1H, Ar—H), 7.02-7.01 (m, 4H, Ar—H), 6.98-6.97 (m, 2H, Ar—H), 6.89-6.81 (m, 5H, Ar—H), 5.46 (s, 1H, Ph-CH), 5.09 (d, J=8.4 Hz, 1H, C1-H_{β}), 4.85 (d, J=11.4 Hz, 1H), 4.77 (d, J=12.6 Hz, 1H), 4.75 (d, J=4.2 Hz, 1H), 4.73 (d, J=4.2 Hz, 1H), 4.68 (d, J=11.4 Hz, 1H), 4.62 (d, J=7.2 Hz, 1H, C1-H_{β}), 4.56 (d, J=12.0 Hz, 1H), 4.46 (d, J=12.0 Hz, 1H), 4.38 (d, J=12.0 Hz, 1H), 4.32 (dd, J=10.8, 8.4 Hz, 1H), 4.21 (d, J=10.8, 8.4 Hz, 1H), 4.15-4.10 (m, 2H), 4.04 (dd, J=11.4, 3.0 Hz, 1H), 3.82 (dd, J=11.4, 1.8 Hz, 1H), 3.78 (s, 3H, —CH₃), 3.61 (ddd, J=9.6, 3.0, 3.0 Hz, 1H), 3.56 (dd, J=3.0, 3.0 Hz, 1H), 3.52-3.45 (m, 3H), 3.18 (ddd, J=9.6, 9.6, 4.8 Hz, 1H), 2.98 (br, 1H, —OH); ¹³C NMR (150 MHz, CDCl₃): δ 167.9, 159.3, 138.4, 137.8, 137.3, 137.2, 133.6, 131.6, 130.5, 129.7, 128.9, 128.4, 128.2, 128.1, 128.0, 127.9, 127.8, 127.5, 127.4, 127.1, 126.0, 123.2, 113.8, 103.4, 101.1, 97.5, 81.3, 79.5, 78.8, 77.8, 75.0, 74.7, 74.5, 74.2, 73.6, 70.8, 68.6, 68.2, 66.2, 55.8, 55.3; HRMS (ESI-TOF) m/e: Calcd for C₅₆H₅₅NO₁₃Na [M+Na]⁺: 972.3565 found 972.3567.

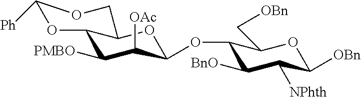

Benzyl [2-O-acetyl-4,6-O-benzylidine-3-O-p-methoxy-benzyl-β-D-mannopyranosyl]-(1→4)-3,6-di-O-benzyl-2-deoxy-2-phthalimido-β-D-glucopyranoside S21. To a stirred solution of disaccharide S20 (936 mg, 0.985 mmol, 1 equiv.) in anhydrous CH₂Cl₂ (12 mL) was added anhydrous pyridine (0.56 mL, 6.90 mmol, 7 equiv.), then trifluoromethanesulfonic anhydride (0.29 mL, 1.72 mmol, 1.75 equiv.) was added dropwise at 0° C. under argon and the mixture was stirred at 0° C. until TLC indicated the disappearance of starting material (2 h). Upon completion, the reaction mixture was diluted with CH₂Cl₂ (20 mL), washed with 0.5 N HCl (20 mL) and brine (10 mL). The separated organic layer was dried over MgSO₄, concentrated in vacuo and the obtained residue was used as is for further reactions. The above-obtained residue was added Bu4NOAc (594 mg, 1.97 mmol, 2 equiv.) and dissolved in toluene (18 mL). The solvent was removed in vacuo and the residue was co-evaporated with toluene twice to remove traces of water. The residue was redissolved in anhydrous toluene (12 mL) and the mixture was sonicated for 8 h. Upon completion, the reaction mixture was diluted with EtOAc (20 mL), washed with water (20 mL) and brine (10 mL). The separated organic layer was dried over MgSO₄ and concentrated. The obtained residue was purified by silica gel column chromatography using EtOAc/hexane (1:2) as eluent to give compound S21 as white powder (913 mg, 93%). R_{f}=0.29 (silica gel, EtOAc:hexane=1:2); ¹H NMR (600 MHz, CDCl₃): δ 7.76-7.49 (m, 4H, Ar—H), 7.48-7.46 (m, 2H, Ar—H), 7.39-7.31 (m, 7H, Ar—H), 7.26-7.22 (m, 3H, Ar—H), 7.10-7.06 (m, 1H, Ar—H), 7.04-7.03 (m, 4H, Ar—H), 6.99-6.98 (m, 2H, Ar—H), 6.90-6.84 (m, 5H, Ar—H), 5.49 (s, 1H, Ph-CH), 5.44 (d, J=3.0 Hz, 1H), 5.09 (d, J=7.8 Hz, 1H, C1-H_{β}), 4.82-4.76 (m, 3H), 4.68 (s, 1H, C1-H_{β}), 4.58 (d, J=12.0 Hz, 1H), 4.49-4.44 (m, 3H), 4.38 (d, J=12.6 Hz, 1H), 4.26-4.19 (m, 2H), 4.17-4.11 (m, 2H), 3.86-3.82 (m, 2H), 3.78 (s, 3H, —CH₃), 3.78-3.76 (m, 1H), 3.57-3.54 (m, 2H), 3.44 (dd, J=10.2, 3.6 Hz, 1H), 3.13 (ddd, J=9.6, 9.6, 4.8 Hz, 1H), 2.15 (s, 3H, —CH₃); ¹³C NMR (150 MHz, CDCl₃): δ 170.2, 167.8, 159.2, 138.5, 137.8, 137.4, 137.1, 133.6, 131.5, 129.7, 129.2, 128.9, 128.5, 128.1, 128.1, 127.9, 127.9, 127.8, 127.7, 127.6, 127.5, 127.1, 126.0, 123.1, 113.8, 101.4, 99.5, 97.3, 79.1, 77.7, 76.9, 75.4, 74.5, 74.3, 73.5, 71.3, 70.7, 69.1, 68.4, 68.3, 66.9, 55.6, 55.2, 21.0; HRMS (ESI-TOF) m/e: Calcd for C₅₈H₅₇NO₁₄Na [M+Na]⁺: 1014.3671 found 1014.3699.

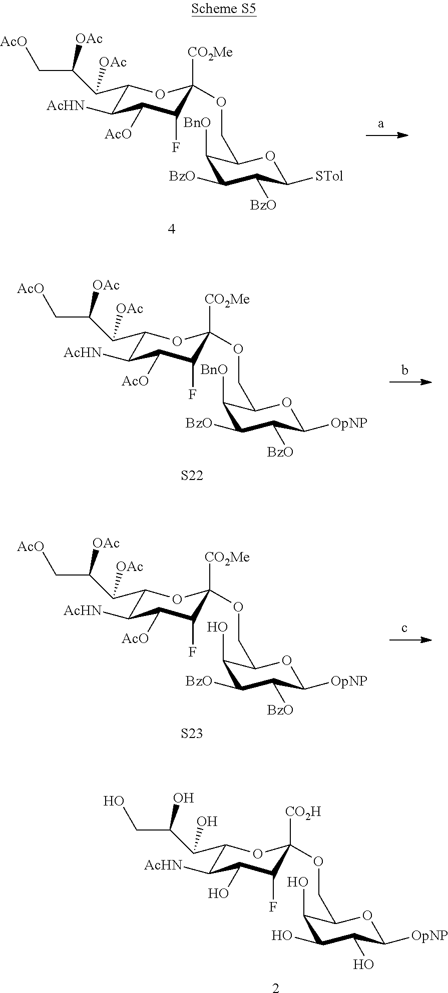

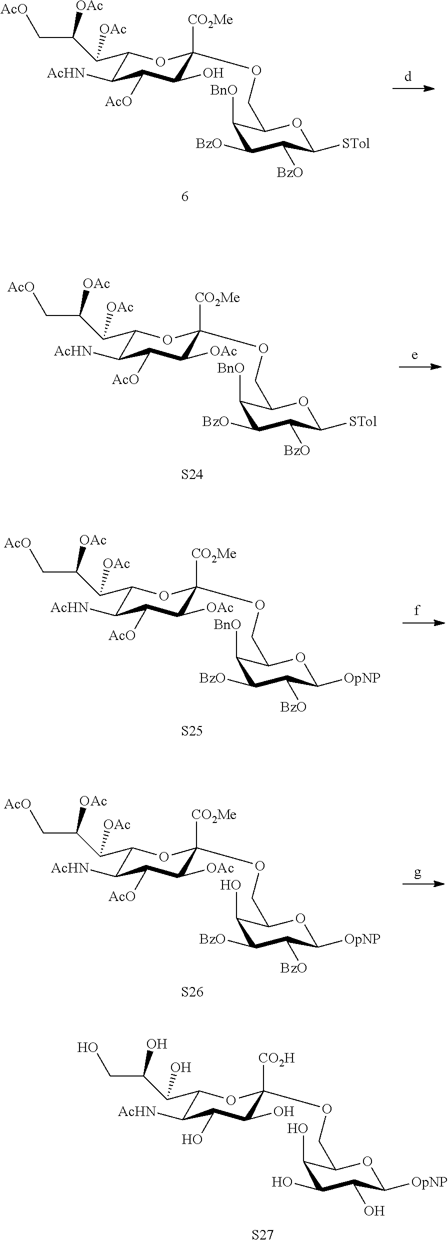

Reagents and conditions in Scheme S5: (a) Br₂, CH₂Cl₂, 0° C., 10 min., then p-nitrophenol, Ag₂O, CH₃CN, 1 h, 73%. (b) NaBrO₃, Na₂S₂O₄, H₂O, EtOAc, r.t., 1.5 h, 93%. (c) LiOH·H₂O, MeOH, r.t., 16 h, 84%. (d) Ac₂O, pyridine, r.t., 16 h, 90%. (e) Br₂, CH₂Cl₂, 0° C., 10 min., then p-nitrophenol, Ag₂O, CH₃CN, 1 h, 74%. (f) NaBrO₃, Na₂S₂O₄, H₂O, EtOAc, r.t., 1.5 h, 97% (g) LiOH·H₂O, MeOH, r.t., 16 h, 94%.

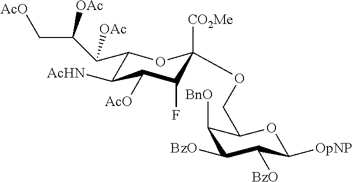

p-Nitrophenyl [methyl 5-acetamido-4,7,8,9-tetra-O-acetyl-3,5-dideoxy-3-fluoro-D-erythro-α-L-manno-non-2-ulopyranosonate]-(2→6)-2,3-di-O-benzoyl-4-O-benzyl-β-D-galactopyranoside S22. To a stirred solution of thioglycoside 4 (115 mg, 0.11 mmol, 1 equiv.) in anhydrous CH₂Cl₂ (2 mL) at 0° C. was added bromine (6.02 μL, 0.12 mmol, 1.1 equiv.). After the reaction mixture was vigorously stirred for 10 min, the solvent was removed in vacuo and the residue was co-evaporated with toluene twice to remove traces of water. Thus obtained residue was dissolved in anhydrous CH₃CN (2 mL) and treated with 4-nitrophenol (25.3 mg, 0.18 mmol, 1.7 equiv.) and Ag₂O (124 mg, 0.53 mmol, 5 equiv.). The reaction mixture was vigorously stirred in the dark under N2 until TLC indicated the disappearance of starting material (1 h). Upon completion, the reaction mixture was diluted with EtOAc (10 mL) and filtered through a pad of Celite. The filtrate was washed twice with satd. aq. NaHCO₃ (4 mL) and brine (3 mL). The organic phase was dried over MgSO₄, filtered, and concentrated in vacuo. The obtained residue was purified by silica gel column chromatography using acetone/toluene (1:2) as eluent to give compound S22 as a white powder (85 mg, 73%). R_{f}=0.46 (silica gel, acetone: toluene=2:3); ¹H NMR (600 MHz, CDCl₃): δ 8.13-8.11 (m, 2H, Ar—H), 7.96-7.94 (m, 2H, Ar—H), 7.91-7.89 (m, 2H, Ar—H), 7.49-7.44 (m, 2H, Ar—H), 7.35-7.30 (m, 6H, Ar—H), 7.24-7.22 (m, 2H, Ar—H), 7.20-7.18 (m, 2H, Ar—H), 7.13-7.11 (m, 1H, Ar—H), 6.09 (dd, J=10.2, 7.8 Hz, 1H), 5.65-5.59 (dd, J=10.2, 3.0 Hz, 1H, C1-H_{β}), 5.35 (d, J=9.6 Hz, 1H), 5.28 (dd, J=9.6, 1.8 Hz, 1H), 5.19 (dd, J=27.0, 10.8 Hz, 1H, sia-C4-H), 5.04 (dd, J=51.0, 1.8 Hz, 1H, sia-C3-H), 4.72-4.67 (m, 2H), 4.44 (dd, J=12.6, 3.6 Hz, 1H), 4.40 (d, J=3.0 Hz, 1H), 4.37 (dd, J=8.4, 6.6 Hz, 1H), 4.31 (d, J=10.8 Hz, 1H), 4.18 (ddd, J=4.2, 4.2, 4.2 Hz, 1H), 4.08 (dd, J=12.6, 6.6 Hz, 1H), 3.89-3.80 (m, 5H, —CH₃), 2.26 (s, 6H, -2CH₃), 2.10 (s, 3H, —CH₃), 1.93 (s, 3H, —CH₃), 1.92 (s, 3H, —CH₃); ¹³C NMR (150 MHz, CDCl₃): δ 171.1, 170.8, 170.4, 170.3, 170.2, 165.5, 165.5, 165.4, 165.3, 161.8, 142.6, 138.0, 133.2, 133.1, 129.8, 129.7, 129.4, 129.0, 128.4, 128.3, 128.2, 128.1, 128.0, 127.5, 125.6, 116.8, 98.8, 98.7, 98.2, 88.1, 86.8, 75.0, 73.9, 73.7, 73.2, 71.6, 69.7, 69.1, 69.0, 67.3, 67.2, 63.4, 63.3, 53.4, 45.1, 23.3, 21.2, 20.8, 20.7, 20.6; ¹⁹F NMR (376 MHz, CDCl₃): δ −215.5; HRMS (ESI-TOF) m/e: Calcd for C₅₃H₅₅FN₂O₂₂Na [M+Na]⁺: 1113.3123 found 1113.3131.

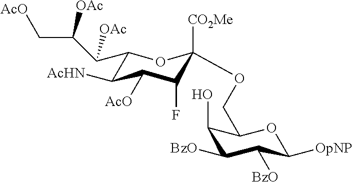

p-Nitrophenyl [methyl 5-acetamido-4,7,8,9-tetra-O-acetyl-3,5-dideoxy-3-fluoro-D-erythro-α-L-manno-non-2-ulopyranosonate]-(2→6)-2,3-di-O-benzoyl-β-D-galactopyranoside S23. To a stirring solution of compound S22 (63.0 mg, 0.058 mmol, 1 equiv.) in EtOAc (0.8 mL) was added NaBrO3 (85%, 39.2 mg, 0.26 mmol, 4.5 equiv.) in H₂O (0.6 mL) followed by a slow addition of Na₂S2O₄ (40.2 mg, 0.23 mmol, 4 equiv.) in H₂O (1.2 mL) at room temperature. The reaction mixture was stirred until TLC indicated the disappearance of the starting material (1.5 h). Upon completion of the reaction, the reaction mixture was diluted with EtOAc (10 mL), washed with 20% aq. Na2S2O₃ (5 mL) and brine (3 mL). The organic phase was dried over MgSO₄, filtered, and concentrated in vacuo, and the obtained residue was purified by silica gel column chromatography using acetone/toluene (3:5) as eluent to give compound S23 as a white powder (54 mg, 93%). R_{f}=0.40 (silica gel, acetone:toluene=2:3); ¹H NMR (600 MHz, CDCl₃): δ 8.13-8.12 (m, 2H, Ar—H), 7.99-7.98 (m, 2H, Ar—H), 7.95-7.94 (m, 2H, Ar—H), 7.50-7.46 (m, 2H, Ar—H), 7.37-7.32 (m, 4H, Ar—H), 7.19-7.18 (m, 2H, Ar—H), 6.05 (dd, J=10.2, 9.8 Hz, 1H), 5.53 (ddd, J=9.0, 6.0, 3.0 Hz, 1H), 5.49-5.45 (m, 2H, C1-H_{β}), 5.36 (d, J=9.0 Hz, 1H), 5.27 (dd, J=9.0, 1.8 Hz, 1H), 5.19 (dd, J=27.0, 11.4 Hz, 1H, sia-C4-H), 5.05 (dd, J=51.6, 1.8 Hz, 1H, sia-C3-H), 4.47 (d, J=3.0 Hz, 1H), 4.37 (dd, J=12.6, 3.0 Hz, 1H), 4.26 (dd, J=10.8, 1.2 Hz, 1H), 4.18 (dd, J=6.6, 6.6 Hz, 1H), 4.13-4.07 (m, 1H), 4.05 (dd, J=12.6, 6.6 Hz, 1H), 3.99-3.98 (m, 2H), 3.85 (s, 3H, —CH₃), 2.99 (br, 1H, —OH), 2.17 (s, 3H, —CH₃), 2.08 (s, 3H, —CH₃), 2.07 (s, 3H, —CH₃), 1.93 (s, 3H, —CH₃), 1.88 (s, 3H, —CH₃); ¹³C NMR (150 MHz, CDCl₃): δ 170.9, 170.8, 170.5, 170.3, 165.6, 165.6, 165.3, 161.8, 142.9, 133.4, 133.3, 129.8, 129.7, 129.2, 129.1, 129.0, 128.4, 128.4, 128.2, 125.6, 125.3, 117.1, 99.0, 98.1, 98.0, 88.3, 87.0, 73.9, 73.3, 71.6, 69.2, 69.1, 69.0, 67.9, 67.2, 66.5, 63.6, 62.9, 53.4, 45.1, 23.3, 21.1, 20.7, 20.6, 20.6; ¹⁹F NMR (376 MHz, CDCl₃): δ −216.0; HRMS (ESI-TOF) m/e: Calcd for C₄₆H₅₀FN₂O₂₂ [M+H]⁺: 1001.2834 found 1001.2833.

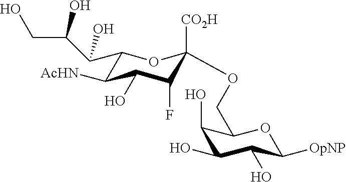

p-Nitrophenyl [methyl 5-acetamido-3,5-dideoxy-3-fluoro-D-erythro-α-L-manno-non-2-ulopyranosonate]-(2→6)-β-D-galactopyranoside 2. To a solution of compound S23 (53 mg, 0.053 mmol, 1 equiv.) in methanol (5 mL) was added LiOH· H₂O (22.2 mg, 0.53 mmol, 10 equiv.) in water (1 mL). After stirring for 16 h at room temperature, the reaction mixture was neutralized with IR-120, filtered, and concentrated in vacuo. The obtained residue was purified by (BIO-RAD) Biogel P-2 column chromatography (eluting with water) to give compound 2 (27 mg, 84%). ¹H NMR (600 MHz, D₂O): δ 8.29-8.28 (m, 2H, Ar—H), 7.30-7.29 (m, 2H, Ar—H), 5.21 (dd, J=51.6, 2.4 Hz, 1H, sia-C3-H), 5.18 (d, J=7.8 Hz, C1-H_{β}), 4.17 (dd, J=9.0, 9.0 Hz, 1H), 4.07-4.00 (m, 3H), 3.91-3.77 (m, 7H), 3.63 (dd, J=12.0, 6.6 Hz, 1H), 3.57 (dd, J=9.0, 1.8 Hz, 1H), 2.04 (s, 3H, —CH₃); ¹³C NMR (150 MHz, D₂O): 67 175.0, 170.8, 161.9, 142.5, 126.1, 116.5, 100.0, 98.9, 98.9, 91.6, 90.3, 73.9, 72.4, 72.4, 71.6, 70.3, 69.7, 69.5, 68.4, 68.1, 63.7, 62.6, 46.8, 46.8, 22.0; ¹⁹F NMR (376 MHz, CDCl₃): δ −217.6; HRMS (ESI-TOF) m/e: Calcd for C₂₃H₃₂FN₂O₁₆ [M+H]⁺: 611.1730 found 611.1732.

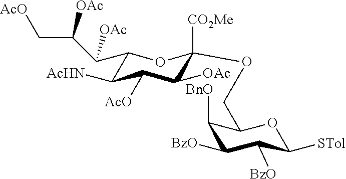

p-Tolyl [methyl 5-acetamido-3,4,7,8,9-penta-O-acetyl-5-deoxy-D-erythro-α-L-gluco-non-2-ulopyranosonate]-(2→6)-2,3-di-O-benzoyl-4-O-benzyl-1-thio-β-D-galactopyranoside S24. To a stirring solution of starting material 6 (174 mg, 0.16 mmol, 1 equiv.) in pyridine (2 mL) was added Ac20 (1 mL). The reaction mixture was vigorously stirred for 16 h at room temperature, then concentrated under high vacuum. The obtained residue was purified by silica gel column chromatography using acetone/toluene (1:2) as eluent to give compound S24 as a white powder (162 mg, 90%). R_{f}=0.49 (silica gel, acetone:toluene=2:3); ¹H NMR (600 MHz, CDCl₃): δ 7.94-7.92 (m, 2H, Ar—H), 7.88-7.87 (m, 2H, Ar—H), 7.48-7.44 (m, 2H, Ar—H), 7.40-7.38 (m, 2H, Ar—H), 7.35-7.27 (m, 6H, Ar—H), 7.23-7.20 (m, 2H, Ar—H), 7.18-7.16 (m, 1H, Ar—H), 7.05-7.03 (m, 2H, Ar—H), 5.80 (dd, J=10.2. 9.6 Hz, 1H), 5.44-5.42 (m, 2H), 5.35-5.29 (m, 3H), 5.25 (dd, J=8.4, 1.8 Hz, 1H), 4.95(d, J=10.2 Hz, 1H, C1-H_{β}), 4.67-4.61 (m, 3H), 4.33 (ddd, J=10.2, 10.2, 10.2 Hz, 1H), 4.25-4.22 (m, 2H), 4.03-3.97 (m, 3H), 3.92-3.89 (m, 1H), 3.75 (s, 3H, —CH₃), 2.29 (s, 3H, —CH₃), 2.15 (s, 3H, —CH₃), 2.07 (s, 3H, —CH₃), 2.00 (s, 3H, —CH₃), 1.95 (s, 3H, —CH₃), 1.94 (s, 3H, —CH₃), 1.89 (s, 3H, —CH₃); ¹³C NMR (150 MHz, CDCl₃): δ 171.0, 170.5, 170.3, 170.0, 169.3, 168.5, 168.0, 165.7, 165.2, 138.4, 137.6, 133.1, 132.9, 132.5, 129.8, 129.7, 129.7, 129.5, 129.2, 129.1, 128.3, 128.2, 128.0, 127.4, 127.2, 98.9, 86.5, 76.9, 75.5, 74.6, 74.3, 72.7, 71.6, 71.5, 68.5, 68.3, 66.9, 62.6, 62.5, 52.7, 48.4, 23.0, 21.1, 20.8, 20.7, 20.6, 20.6, 20.5; HRMS (ESI-TOF) m/e: Calcd for C₅₆H₆₁NO₂₁SNa [M+Na]⁺: 1138.3349 found 1138.3358.

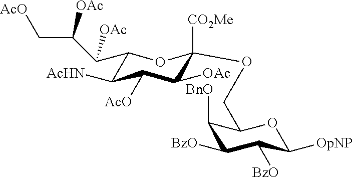

p-Nitrophenyl [methyl 5-acetamido-3,4,7,8,9-penta-O-acetyl-5-deoxy-D-erythro-α-L-gluco-non-2-ulopyranosonate]-(2→6)-2,3-di-O-benzoyl-4-O-benzyl-β-D-galactopyranoside S25. To a stirring solution of thioglycoside S24 (132 mg, 0.12 mmol, 1 equiv.) in anhydrous CH₂Cl₂ (2.5 mL) at 0° C. was added bromine (6.67 μL, 0.13 mmol, 1.1 equiv.). After vigorously stirring for 10 min, the solvent was removed in vacuo and the residue was co-evaporated with toluene twice to remove traces of water. The obtained residue was dissolved in anhydrous CH₃CN (2.5 mL) and treated with 4-nitrophenol (28.0 mg, 0.20 mmol, 1.7 equiv.) and Ag₂O (137 mg, 0.59 mmol, 5 equiv.). The reaction mixture was vigorously stirred in the dark under N2 until TLC indicated the disappearance of starting material (1 h). Upon completion of the reaction, the reaction mixture was diluted with EtOAc (12 mL), and filtered through a pad of Celite. The filtrate was washed twice with satd. aq. NaHCO₃ (5 mL) and brine (3 mL). The organic phase was dried over MgSO₄, filtered, and concentrated in vacuo, and the obtained residue was purified by silica gel column chromatography using acetone/toluene (3:5) as eluent to give compound S25 as a white powder (99 mg, 74%). R_{f}=0.51 (silica gel, acetone: toluene=2:3); ¹H NMR (600 MHz, CDCl₃): δ 8.15-8.13 (m, 2H, Ar—H), 7.95-7.92 (m, 4H, Ar—H), 7.50-7.44 (m, 2H, Ar—H), 7.36-7.31 (m, 6H, Ar—H), 7.24-7.19 (m, 2H, Ar—H), 7.17-7.15 (m, 3H, Ar—H), 6.10 (dd, J=10.2, 7.8 Hz, 1H), 5.54 (dd, J=10.2, 3.0 Hz, 1H), 5.52 (d, J=7.8 Hz, 1H, C1-H_{β}), 5.48 (ddd, J=10.2, 7.2, 3.0 Hz, 1H), 5.39 (d, J=10.2 Hz, 1H), 5.31-5.29 (m, 2H), 5.23 (dd, J=9.6, 1.8 Hz, 1H), 4.73 (dd, J=10.8, 2.4 Hz, 1H), 4.71-4.65 (m, 2H), 4.32-4.30 (m, 3H), 4.19 (dd, J=7.2, 7.2 Hz, 1H), 3.97 (dd, J=12.6, 7.2 Hz, 1H), 3.88-3.86 (m, 2H), 3.83 (s, 3H, —CH₃), 2.24 (s, 3H, —CH₃), 2.12 (s, 3H, —CH₃), 2.01 (s, 3H, —CH₃), 1.98 (s, 3H, —CH₃), 1.90 (s, 3H, —CH₃), 1.88 (s, 3H, —CH₃); ¹³C NMR (150 MHz, CDCl₃): δ 171.0, 170.7 (2C), 170.0, 169.6, 168.5, 168.3, 165.7, 165.3, 161.7, 142.7, 137.9, 133.3, 133.2, 129.9, 129.7, 129.3, 128.9, 128.4, 128.3, 128.2, 128.0, 127.6, 125.7, 116.8, 99.7, 98.4, 75.1, 74.1, 74.0, 73.9, 72.8, 71.6, 71.1, 69.7, 67.6, 67.0, 63.8, 63.3, 52.9, 48.4, 23.0, 20.9, 20.8, 20.7, 20.6; HRMS (ESI-TOF) m/e: Calcd for C₅₅H₅₉N₂O₂₄ [M+H]⁺: 1131.3452 found 1131.3440.

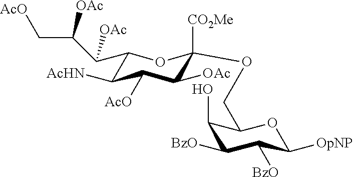

p-Nitrophenyl [methyl 5-acetamido-3,4,7,8,9-penta-O-acetyl-5-deoxy-D-erythro-α-L-gluco-non-2-ulopyranosonate]-(2→6)-2,3-di-O-benzoyl-β-D-galactopyranoside S26. To a stirring solution of compound S25 (91.0 mg, 0.080 mmol, 1 equiv.) in EtOAc (1.2 mL) was added NaBrO₃ (54.6 mg, 0.36 mmol, 4.5 equiv.) in H₂O (0.9 mL) followed by a slow addition of Na₂S₂O₄ (85%, 56.0 mg, 0.32 mmol, 4 equiv.) in H₂O (1.8 mL) at room temperature. The reaction mixture was stirred until TLC indicated the disappearance of the starting material (1.5 h). Upon completion, the reaction mixture was diluted with EtOAc (15 mL), washed with 20% aq. Na₂S₂O₃ (7 mL) and brine (4 mL). The organic phase was dried over MgSO₄, filtered, and concentrated in vacuo. The obtained residue was purified by silica gel column chromatography using acetone/toluene (3:5) as eluent to give compound S26 as a white powder (81 mg, 97%). R_{f}=0.23 (silica gel, acetone: toluene=2:3); ¹H NMR (600 MHz, CDCl₃): δ 8.18-8.16 (m, 2H, Ar—H), 8.00-7.99 (m, 2H, Ar—H), 7.94-7.93 (m, 2H, Ar—H), 7.51-7.46 (m, 2H, Ar—H), 7.38-7.32 (m, 4H, Ar—H), 7.15-7.13 (m, 2H, Ar—H), 6.07 (dd, J=10.2, 7.8 Hz, 1H), 5.46-5.34 (m, 5H, C1-H_{β}), 5.30 (d, J=10.2 Hz, 1H), 5.22 (dd, J=7.8, 1.8 Hz, 1H), 4.60 (dd, J=10.8, 1.8 Hz, 1H), 4.43 (d, J=3.0 Hz, 1H), 4.33 (dd, J=12.6, 2.4 Hz, 1H), 4.26 (ddd, J=10.2, 10.2, 10.2 Hz, 1H), 4.12 (dd, J=6.6, 6.6 Hz, 1H), 4.05 (dd, J=10.2, 6.6 Hz, 1H), 3.99 (dd, J=10.2, 6.6 Hz, 1H), 3.93 (dd, J=12.6, 7.2 Hz, 1H), 3.83 (s, 3H, —CH₃), 3.17 (br, 1H, —OH), 2.14 (s, 3H, —CH₃), 2.05 (s, 3H, —CH₃), 2.02 (s, 3H, —CH₃), 2.00 (s, 3H, —CH₃), 1.87 (s, 3H, —CH₃), 1.85 (s, 3H, —CH₃); ¹³C NMR (150 MHz, CDCl₃): δ 171.1, 171.0, 170.4, 170.0, 169.9, 168.8, 167.9, 165.8, 165.2, 161.7, 142.9, 133.4, 133.3, 129.9, 129.7, 129.3, 129.1, 128.4, 128.4, 125.8, 116.9, 99.1, 98.9, 74.0, 73.9, 73.0, 71.1, 70.6, 69.2, 68.7, 67.2, 66.5, 63.0, 62.5, 53.0, 48.5, 23.0, 20.8, 20.7, 20.6, 20.6; HRMS (ESI-TOF) m/e: Calcd for C₄₈H₅₃N₂O₂₄ [M+H]⁺: 1041.2983 found 1041.2976.

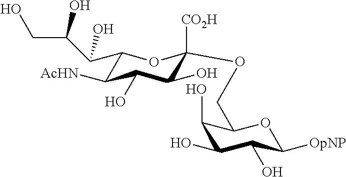

p-Nitrophenyl [5-acetamido-5-deoxy-D-erythro-α-L-gluco-non-2-ulopyranosonate]-(2→6)-β-D-galactopyranoside S27. To a solution of compound S26 (75 mg, 0.072 mmol, 1 equiv.) in methanol (5 mL) was added LiOH·H₂O (30 mg, 0.72 mmol, 10 equiv.) in water (1 mL). After stirring for 16 h at room temperature, the reaction mixture was neutralized with IR-120, filtered, and concentrated. The obtained residue was purified by (BIO-RAD) Biogel P-2 column chromatography using water as eluent to give compound S27 (41 mg, 94%). ¹H NMR (600 MHz, D₂O): δ 8.29-8.28 (m, 2H, Ar—H), 7.28-7.27 (m, 2H, Ar—H), 5.20 (d, J=7.2 Hz, C1-H_{β}), 4.09-4.06 (m, 2H), 4.01 (dd, J=10.8, 7.8 Hz, 1H), 3.92-3.79 (m, 6H), 3.71 (d, J=10.8 Hz, 1H), 3.64-3.60 (m, 2H), 3.53 (d, J=9.6 Hz, 1H), 3.49 (d, J=9.6 Hz, 1H), 2.02 (s, 3H, —CH₃); ¹³C NMR (150 MHz, D₂O): δ 174.8, 173.0, 161.9, 142.5, 126.1, 116.4, 99.9, 98.1, 76.1, 73.9, 73.5, 72.4, 72.2, 71.5, 70.3, 68.4, 68.0, 63.0, 62.6, 50.7, 21.9; HRMS (ESI-TOF) m/e: Calcd for C₂₃H₃₃N₂O₁₇ [M+H]⁺: 609.1774 found 609.1774.

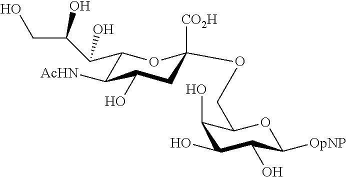

p-Nitrophenyl [5-acetamido-5-deoxy-D-glycero-α-D-galacto-non-2-ulopyranosylonate]-(2→6)-β-D-galactopyranoside 1. Neu5Ac-α2,6-Gal-pNP: Neu5Ac-α2,6-Gal-pNP was synthesized by mixing pNP-β-Gal (1.0 mmol), sialic acid (1.2 mmol), cytidine triphosphate (1.2 mmol), CMP-sialic acid synthetases (CSS, 12 U), pyrophosphatase (PPA, 1U) and α-2,6-sialyltransferase (SiaT, 15U) in 15 mL Tris buffer (pH 7.0) with 5 mM MgCl₂ and 5 mM MnCl₂. After removal of the proteins by heating and centrifugation, the product was purified by (BIO-RAD) Biogel P-2 column chromatography using water as eluent. The fractions containing Neu5Ac-α2,6-Gal-pNP were collected, and lyophilized to give compound 1 (50%). ¹H NMR (600 MHz, D₂O): δ 8.33-8.32 (m, 2H, Ar—H), 7.31-7.29 (m, 2H, Ar—H), 5.23 (d, J=7.8 Hz, C1-H_{β}), 4.06-4.04 (m, 2H), 3.99 (dd, J=10.2, 8.4 Hz, 1H), 3.90-3.86 (m, 3H), 3.83 (dd, J=10.2, 3.6 Hz, 1H), 3.80-3.76 (m, 1H), 3.73-3.69 (m, 3H), 3.65-3.62 (m, 2H), 3.57 (d, J=8.4 Hz, 1H), 2.79 (dd, J=12.6, 4.8 Hz, 1H), 2.04 (s, 3H, —CH₃), 1.68 (dd, J=12.6, 12.0 Hz, 1H); ¹³C NMR (150 MHz, D₂O): δ 174.6, 173.1, 161.4, 142.1, 125.7, 116.0, 99.8, 99.4, 73.6, 72.1, 71.9, 71.3, 69.8, 68.0, 67.7, 67.7, 62.5, 62.2, 51.4, 39.8, 21.6; HRMS (ESI-TOF) m/e: Calcd for C₂₃H₃₃N₂O₁₆ [M+H]⁺: 593.1825 found 593.1825.

### EXAMPLE 2

### Stability Against Sialidase-Catalyzed Hydrolysis and Analysis of Sialidase Inhibition³¹

### Materials

β-Galactosidase of Aspergillus oryzae (G5160) and sialidases from Vibrio cholerae (11080725001) and Clostridium perfringens (11585886001) were received from Sigma Aldrich.

### Enzymatic Assays for Sialidases

The assays were carried out at 37° C. in duplicates in 96-well plates in a final volume of 50 μL containing a substrate (0-20 mM), and β-galactosidase (100 mU). The assay conditions for the two sialidases were as follows: C. perfringens (1 mU), sodium acetate buffer (50 mM) pH 5.0 and CaCl₂ (10 mM); V. cholerae (2 mU), sodium acetate buffer (50 mM) pH 5.5, CaCl₂ (10 mM) and NaCl (150 mM). The reactions were carried out for 40 min to 2 hrs for C. perfringens and overnight for V. cholerae. The assays were stopped by adding 65 μL of CAPS buffer (N-cyclohexyl-3-aminopropane sulfonic acid, 0.5 M, pH 10.5). The amount of the para-nitrophenolate formed was determined by measuring the A_{405 nm} of the reaction mixtures using a microplate reader. Three compounds (Neu5Ac-α2,6-GalβpNP, 3F^{ax}-Neu5Ac-α2,6-GalβpNP and 30H^{eq}-Neu5Ac-α2,6-GalβpNP) were tested as substrates for the enzymes. All three compounds have the background absorbance of A_{405 nm} at 20 mM after incubation for 1 hr at 37° C. in the absence of sialidase. The absorbance of the three compounds Neu5Ac-α2,6-GalβpNP, 3F^{ax}-Neu5Ac-α2,6-GalβpNP and 30H^{eq}-Neu5Ac-α2,6-GalβpNP were 0.044, 0.129 and 0.072, respectively. The standard curve of pNP was determined by series two-fold dilution of 0.35 mM of pNP, then graphing the A_{405 nm} against the concentration of pNP resulted in the standard curve of pNP.

### Inhibition Assays for Sialidases

The assays were carried out at 37° C. in duplicates in 96-well plates in a final volume of 50 μL containing the substrate Neu5Ac-α2,6-GalβpNP (0.6 mM), β-galactosidase (100 mU), and sialidase in the absence or presence of an inhibitor at varied concentrations (0-20 mM). Reactions were allowed to proceed for 40 min to 2 hrs for C. perfringens and overnight for V. cholera. The assays were stopped by adding CAPS buffer (65 μL, 0.5 M, pH 10.5). The amount of the para-nitrophenolate formed was determined by measuring the A_{405 nm} of the reaction mixture using a microplate reader (FIG. 1).

### EXAMPLE 3

### Preparation of Homogeneous mAb Modified With 3F^{ax}-Neu5Ac and Neu5Ac to Study the Effect on Binding to FcγRIIIa by Surface Plasmon Resonance (SPR) Analysis

### Expression of Enzymes

The endo-glycosidases Endo-S, Endo-S2, Endo-S2 mutant (D184Q), and the α-L-fucosidase from Bacteroides fragilis NCTC 9343 were expressed in Escherichia coli and the purification of enzymes was performed using Ni-NTA agarose beads.

### Preparation of Mono-GleNAc-Rituximab

As described previously,³² Rituximab (3.0 mg; Rituxan® Roche) in a Tris-HCl buffer (50 mM, pH 7.4, 1.5 mL) was incubated with Endo-S (120 μg), Endo-S2 (240 μg) and BfFucH (4.5 mg) at 37° C. for 24 h. LC-MS and SDS-PAGE analyses indicated the complete cleavage of the N-glycans on the heavy chain. The reaction mixture was subjected to affinity chromatography on a column of protein A-agarose resin (1 mL; GE Healthcare) pre-equilibrated with a Tris-HCl buffer (50 mM, pH 7.4). Then, the column was washed with a Tris-HCl buffer (50 mM, pH 7.4, 20 mL). The bound IgG was released with glycine-HCl (100 mM, pH 3.0, 10 mL), and the elution fractions were immediately neutralized with Tris-HCl buffer (1.0 M, pH 8.3). The fractions containing the antibody were combined and concentrated by centrifugal filtration (Amicon Ultra centrifugal filter, Millipore, Billerica, MA) to give mono-GlcNAc Rituximab (2.4 mg). The product was trypsinized, and the glycopeptides, TKPREEQYNSTYR (m/z=1391.58) and EEQYNSTYR (m/z=1873.88) were analyzed using nanospray LC/MS to confirm the glycosylation pattern of mono-GlcNAc.

### Transglycosylation of Mono-GleNAc Rituximab With Glycan Oxazolines

A glycan oxazoline was added to the mixture of an Endo-S2 D184Q and Mono-GlcNAc Rituximab in 50 mM Tris buffer (pH 7.4). The solution was incubated for 30 min at 37° C. Then, the reaction mixture was purified with protein-A affinity column, followed by an anion exchange column of Capto Q (GE Healthcare) to collect the desired product.

### SDS-PAGE Detection of Glycoengineered Herceptin Antibodies

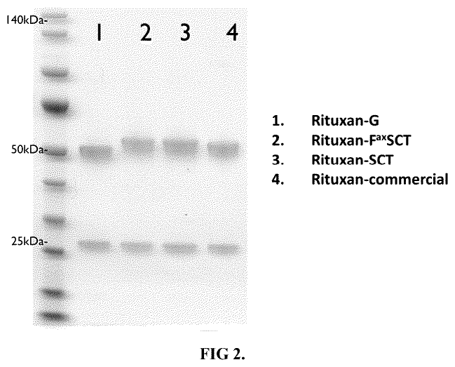

All the SDS-PAGE analyses were performed with NuPAGE® Novex® 4-12% Bis-Tris gel (Invitrogen) in MOPS buffer with 2-mercaptoethanol present in samples (FIG. 2).

### MS Spectrometry Analysis of Glycoengineered mAb

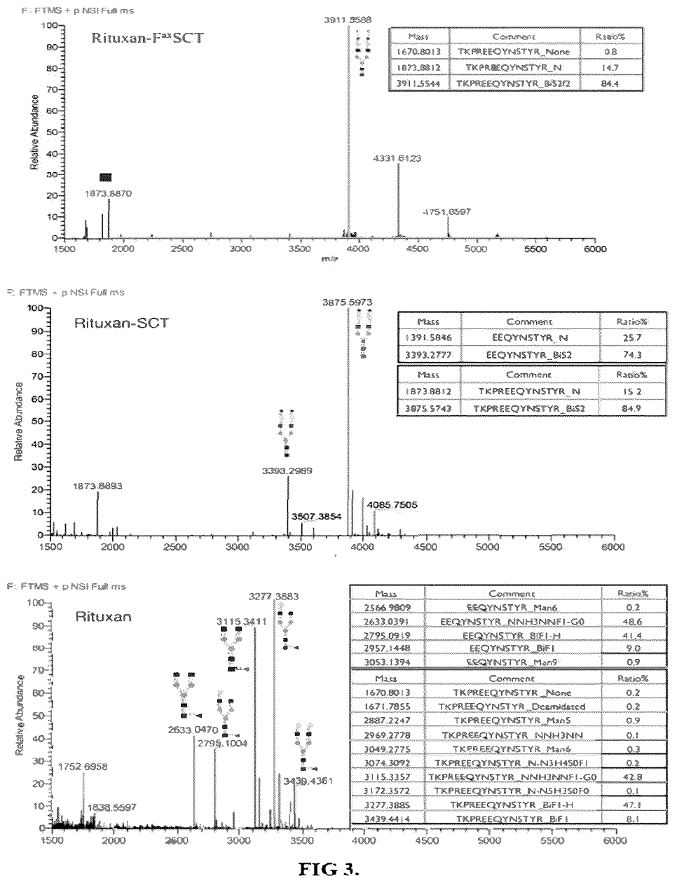

For the analysis of trypsinized glycopeptides, high resolution and high mass accuracy nanoflow LC-MS/MS experiments were performed on a LTQFT Ultra (linear quadrupole ion trap Fourier transform ion cyclotron resonance) mass spectrometer (Thermo Electron, San Jose, CA) equipped with a nanoelectrospry ion source (New Objective, Inc.), an Agilent 1100 Series binary high-performance liquid chromatography pump (Agilent Technologies, Palo Alto, CA), and a Famos autosampler (LC Packings, San Francisco, CA). The digestion solution (6 μL) was injected at the 10 μL/min flow rate to a self-packed precolumn (150 μm I.D.×20 mm, 5 μm, 100 Å). The chromatographic separation was performed on a self-packed reversed phase C18 nano-column (75 μm I.D.×300 mm, 5 μm, 100 Å) using 0.1% formic acid in water as a mobile phase A and 0.1% formic acid in 80% acetonitrile as mobile phase B operated at 300 nL/min flow rate. Survey of full-scan MS conditions: mass range m/z 320-2000, resolution 100,000 at m/z 400. The ten most intense ions were sequentially isolated for MS2 by LTQ. Electrospray voltage was maintained at 1.8 kV and the capillary temperature was set at 200° C. (FIG. 3 and Table 3).

### Surface Plasmon Resonance (SPR) Analysis

All the SPR experiments were performed with the single cycle kinetic method by BIACORE T200 at 25° C. using HBS-EP (10 mM HEPES pH7.4, 0.15M NaCl, 3mMEDTA, 0.005% surfactant P20) as running buffer. FcγRIIIa was transfected into HEK-293 cells to express the complex-type glycosylated recombinant protein as analyte. For the analysis of Rituximabs binding to FcγRIIIa receptor, antihuman Fab antibodies in human Fab capture kit (GE Healthcare) were immobilized onto both the reference and active channels of CM5 sensor chip, and then Rituximabs were captured on the active channel for interacting with the serial dilutions of FcγRIIIa analyte (2.5, 5, 10, 20, 40 nM for 2,6-FluoSCT and 2,6-SCT; 8, 24, 72, 216, 648 nM for the commercial Rituximabs) at 30 μl/min for association of 240 seconds followed by dissociation time of 420 seconds. Rituximab data were processed with double referencing for background subtraction. Data of Rituximabs was fitted to 1:1 Langmuir binding model in BiaEvaluation software (GE Healthcare) to obtain the kinetic/affinity constants (Table 4). Analyzed antibodies were captured by the Human Fab capture kit and detected with the single cycle kinetic method.

TABLE 3
Calibration of N-glycan Relative Abundance³³

| Rit-F^{ax}SCT | Rit-SCT | |
|---|---|---|
| Concentrate | Concentrate | % |
| 1.0 | 0.0 | none |
| 1.7 | 1.8 | Rit-GlcNa |
| 0.0 | 98.2 | Rit-SCT |
| 97.3 | 0.0 | Rit-FluoSCT |

TABLE 4
Binding Avidity of Glycoengineered Rituximab IgG1 to FcγRIIIa Measured by SPR

| Sample | ka (1/Ms) | kd (1/s) | KD (M) | Rmax (RU) | Fold |
|---|---|---|---|---|---|
| Rituximab | 2.31E+05 | 0.07054 | 3.06E−07 | 32.33 | 1-fold |
| 2,6-F^{ax}SCT | 2.44E+05 | 0.001996 | 8.18E−09 | 71.28 | 37.4-fold |
| 2,6-SCT | 2.68E+05 | 0.002059 | 7.67E−09 | 60.64 | 39.9-fold |

### EXAMPLE 4

### Relative Reactivity Values (RRV) of Compounds 4 and 18

The RRVs were measured in triplicates by following the experimental procedure reported previously.³⁴ The RRV (2053) of disaccharide donor 4 was measured against a competition reference donor S34²³ (RRV=1791). The RRV (537) of disaccharide donor 18 was measured against a competition reference donor S²³ (RRV=286).

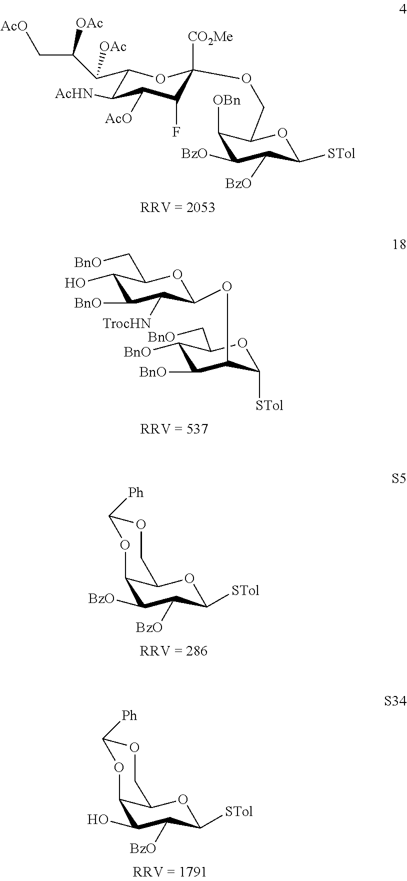

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

Further, from the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### REFERENCES

(1) Varki, A. Sialic acids in human health and disease. Trends Mol. Med. 2008, 14, 351.
(2) (a) Liu, Y.-C.; Yen, H.-Y.; Chen, C.-Y.; Chen, C.-H.; Cheng, P.-F.; Juan, Y.-H.; Chen, C.-H.; Khoo, K.-H.; Yu, C.-J.; Yang, P.-C.; Hsu, T.-L.; Wong, C.-H. Sialylation and fucosylation of epidermal growth factor receptor suppress its dimerization and activation in lung cancer cells. Proc. Natl. Acad. Sci. U.S.A. 2011, 108, 11332. (b) Yen, H.-Y.; Liu, Y.-C.; Chen, N.-Y.; Tsai, C.-F.; Wang, Y.-T.; Chen, Y.-J.; Hsu, T.-L.; Yang, P.-C.; Wong, C.-H. Effect of sialylation on EGFR phosphorylation and resistance to tyrosine kinase inhibition. Proc. Natl. Acad. Sci. U.S.A. 2015, 112, 6955.
(3) (a) Ashwell, G.; Harford, J. Carbohydrate-specific receptors of the liver. Annu. Rev. Biochem. 1982, 51, 531. (b) Weigel, P. H.; Yik, J. H. Glycans as endocytosis signals: the cases of the asialoglycoprotein and hyaluronan/chondroitin sulfate receptors. Biochim. Biophys. Acta 2002, 1572, 341.
(4) (a) Wang, Z.; Chinoy, Z. S.; Ambre, S. G.; Peng, W.; McBride, R.; de Vries, R. P.; Glushka, J.; Paulson, J. C.; Boons, G. J. A general strategy for the chemoenzymatic synthesis of asymmetrically branched N-glycans. Science 2013, 341, 379. (b) Shivatare, S. S.; Chang, S. H.; Tsai, T. I.; Tseng, S. Y.; Shivatare, V. S.; Lin, Y. S.; Cheng, Y. Y.; Ren, C. T.; Lee, C. C.; Pawar, S.; Tsai, C. S.; Shih, H. W.; Zeng, Y. F.; Liang, C. H.; Kwong, P. D.; Burton, D. R.; Wu, C. Y.; Wong, C. H. Modular synthesis of N-glycans and arrays for the hetero-ligand binding analysis of HIV antibodies. Nat. Chem. 2016, 8, 338. (c) Li, L.; Liu, Y.; Ma, C.; Qu, J.; Calderon, A. D.; Wu, B.; Wei, N.; Wang, X.; Guo, Y.; Xiao, Z.; Song, J.; Sugiarto, G.; Li, Y.; Yu, H.; Chen, X.; Wang, P. G. Efficient chemoenzymatic synthesis of an N-glycan isomer library. Chem. Sci. 2015, 6, 5652.
(5) Li, C.; Wang, L.-X. Chemoenzymatic Methods for the Synthesis of Glycoproteins. Chem. Rev. 2018, 118, 8359.
(6) (a) Lin, C.-W.; Tsai, M.-H.; Li, S.-T.; Tsai, T.-I.; Chu, K.-C.; Liu, Y.-C.; Lai, M.-Y.; Wu, C.-Y.; Tseng, Y.-C.; Shivatare, S. S.; Wang, C.-H.; Chao, P.; Wang, S.-Y.; Shih, H.-W.; Zeng, Y.-F.; You, T.-H.; Liao, J.-Y.; Tu, Y.-C.; Lin, Y.-S.; Chuang, H.-Y.; Chen, C.-L.; Tsai, C.-S.; Huang, C.-C.; Lin, N.-H.; Ma, C.; Wu, C.-Y.; Wong, C.-H. A common glycan structure on immunoglobulin G for enhancement of effector functions. Proc. Natl. Acad. Sci. U.S.A. 2015, 112, 10611. (b) Tsai, T.-I.; Li, S.-T.; Liu, C.-P.; Chen, K. Y.; Shivatare, S. S.; Lin, C.-W.; Liao, S.-F.; Lin, C.-W.; Hsu, T.-L.; Wu, Y.-T.; Tsai, M.-H.; Lai, M.-Y.; Lin, N.-H.; Wu, C.-Y.; Wong, C.-H. An Effective Bacterial Fucosidase for Glycoprotein Remodeling. ACS Chem. Biol. 2017, 12, 63. (c) Liu, C.-P.; Tsai, T.-I.; Cheng, T.; Shivatare, V. S.; Wu, C.-Y.; Wu, C.-Y.; Wong, C.-H. Glycoengineering of antibody (Herceptin) through yeast expression and in vitro enzymatic glycosylation. Proc. Natl. Acad. Sci. U.S.A. 2018, 115, 720.
(7) (a) Gantt, R.; Millner, S.; Binkley, S. B. Inhibition of N-Acetylneuraminic Acid Aldolase by 3-Fluorosialic Acid. Biochemistry 1964, 3, 1952. (b) Hagiwara, T.; Kijima-Suda, I.; Ido, T.; Ohrui, H.; Tomita, K. Inhibition of bacterial and viral sialidases by 3-fluoro-N-acetylneuraminic acid. Carbohydr. Res. 1994, 263, 167. (c) Burkart, M. D.; Zhang, Z.; Hung, S.-C.; Wong, C.-H. A New Method for the Synthesis of Fluoro-Carbohydrates and Glycosides Using Selectfluor. J. Am. Chem. Soc. 1997, 119, 11743. (d) D. Burkart, M.; P. Vincent, S.; Wong, C.-H. An efficient synthesis of CMP-3-fluoroneuraminic acid. Chem. Commun. 1999, 1525. (e) Burkart, M. D.; Vincent, S. P.; Duffels, A.; Murray, B. W.; Ley, S. V.; Wong, C.-H. Chemo-enzymatic synthesis of fluorinated sugar nucleotide: useful mechanistic probes for glycosyltransferases. Bioorg. Med. Chem. 2000, 8, 1937. (f) Sun, X.-L.; Kanie, Y.; Guo, C.-T.; Kanie, O.; Suzuki, Y.; Wong, C.-H. Syntheses of C-3-Modified Sialylglycosides as Selective Inhibitors of Influenza Hemagglutinin and Neuraminidase. Eur. J. Org. Chem. 2000, 2643. (g) Rillahan, C. D.; Antonopoulos, A.; Lefort, C. T.; Sonon, R.; Azadi, P.; Ley, K.; Dell, A.; Haslam, S. M.; Paulson, J. C. Global metabolic inhibitors of sialyl- and fucosyltransferases remodel the glycome. Nat. Chem. Biol. 2012, 8, 661.
(8) Ishiwata, K.; Ido, T.; Nakajima, T.; Ohrui, H.; Kijima-Suda, I.; Itoh, M. Tumor uptake study of 18F-labeled N-acetylneuraminic acids. International journal of radiation applications and instrumentation. Int. J. Rad. Appl. Instrum. B 1990, 17, 363.
(9) (a) Watts, A. G.; Damager, I.; Amaya, M. L.; Buschiazzo, A.; Alzari, P.; Frasch, A. C.; Withers, S. G. Trypanosoma cruzi Trans-sialidase Operates through a Covalent Sialyl—Enzyme Intermediate: Tyrosine Is the Catalytic Nucleophile. J. Am. Chem. Soc. 2003, 125, 7532. (b) Buchini, S.; Gallat, F. X.; Greig, I. R.; Kim, J. H.; Wakatsuki, S.; Chavas, L. M.; Withers, S. G. Tuning Mechanism-Based Inactivators of Neuraminidases: Mechanistic and Structural Insights. Angew. Chem. Int. Ed. Engl. 2014, 53, 3382. (c) Kim, J. H.; Resende, R.; Wennekes, T.; Chen, H. M.; Bance, N.; Buchini, S.; Watts, A. G.; Pilling, P.; Streltsov, V. A.; Petric, M.; Liggins, R.; Barrett, S.; McKimm-Breschkin, J. L.; Niikura, M.; Withers, S. G. Mechanism-Based Covalent Neuraminidase Inhibitors with Broad-Spectrum Influenza Antiviral Activity. Science 2013, 340, 71.
(10) Tsai, C. S.; Yen, H. Y.; Lin, M. I.; Tsai, T. I.; Wang, S. Y.; Huang, W. I.; Hsu, T. L.; Cheng, Y. S.; Fang, J. M.; Wong, C.-H. Cell-permeable probe for identification and imaging of sialidases. Proc. Natl. Acad. Sci. U.S.A. 2013, 110, 2466.
(11) (a) Chokhawala, H. A.; Cao, H.; Yu, H.; Chen, X. Enzymatic Synthesis of Fluorinated Mechanistic Probes for Sialidases and Sialyltransferases. J. Am. Chem. Soc. 2007, 129, 10630. (b) McArthur, J. B.; Yu, H.; Zeng, J.; Chen, X. Converting Pasteurella multocida α2-3-sialyltransferase 1 (PmST1) to a regioselective α2,6-sialyltransferase by saturation mutagenesis and regioselective screening. Org. Biomol. Chem., 2017, 15, 1700.
(12) Petrie, C. R.; Sharma, M.; Simmons, O. D.; Korytnyk, W. Synthesis of analogs of N-acetylneuraminic acid and their effect on CMP-sialate synthase. Carbohydr. Res. 1989, 186, 326.
(13) Nakajima, T.; Hori, H.; Ohrui, H.; Meguro, H.; Ido, T. Synthesis of N-Acetyl-3-fluoro-neuraminic Acids Agric. Biol. Chem. 1988, 52, 1209.
(14) Watts, A. G.; Withers, S. G. The synthesis of some mechanistic probes for sialic acid processing enzymes and the labeling of a sialidase from Trypanosoma rangeli. Can. J. Chem 2004, 82, 1581.
(15) Hayashi, T.; Kehr, G.; Gilmour R. Stereospecific α-sialylation by site-selective fluorination. Angew. Chem. Int. Ed. Engl. 10.1002/anie.201812963.
(16) Okamoto, K.; Kondo, T.; Goto, T. An effective synthesis of α-glycosides of N-acetylneuraminic acid derivatives by use of 2-deoxy-2β-halo-3β-hydroxy-4,7,8,9-tetra-O-acetyl-N-acetylneuraminic acid methyl ester. Tetrahedron 1987, 43, 5919.
(17) Bennua-Skalmowski, B.; Vorbrüggen, H. A facile conversion of primary or secondary alcohols with n-perfluorobutane-sulfonyl fluoride/1,8-diazabicyclo[5.4.0]undec-7-ene into their corresponding fluorides. Tetrahedron Lett. 1995, 36, 2611.
(18) Cao, H.; Li, Y.; Lau, K.; Muthana, S.; Yu, H.; Cheng, J.; Chokhawala, H. A.; Sugiarto, G.; Zhang, L.; Chen, X. Sialidase substrate specificity studies using chemoenzymatically synthesized sialosides containing C5-modified sialic acids. Org. Biomol. Chem., 2009, 7, 5137.
(19) Orlova, A. V.; Shpirt, A. M.; Kulikova, N. Y.; Kononov, L. O. N,N-Diacetylsialyl chloride—a novel readily accessible sialyl donor in reactions with neutral and charged nucleophiles in the absence of a promoter. Carbohydr. Res. 2010, 345, 721.
(20) Okamoto, K.; Kondo, T.; Goto. T. Functionalization of 2-Deoxy-2,3-dehydro-N-acetylneuraminic Acid Methyl Ester. Bull. Chem. Soc. Jpn. 1987, 60, 631.
(21) Pascolutti, M.; Madge, P. D.; Thomson, R. J.; von Itzstein, M. Access to 3-O-Functionalized N-Acetylneuraminic Acid Scaffolds. J. Org. Chem. 2015, 80, 7746.
(22) Wang, C.-C.; Lee, J.-C.; Luo, S.-Y.; Kulkarni, S. S.; Huang, Y.-W.; Lee, C.-C.; Chang, K.-L.; Hung, S.-C. Regioselective one-pot protection of carbohydrates. Nature 2007, 446, 896.
(23) Zhang, Z.; Ollmann, I. R.; Ye, X.-S.; Wischnat, R.; Baasov, T.; Wong, C.-H. Programmable One-Pot Oligosaccharide Synthesis. J. Am. Chem. Soc. 1999, 121, 734.
(24) Huang, L.; Huang, X. Highly Efficient Syntheses of Hyaluronic Acid Oligosaccharides. Chem. Eur. J. 2007, 13, 529.
(25) Shivatare, S. S.; Chang, S.-H.; Tsai, T.-I; Ren, C.-T.; Chuang, H.-Y.; Hsu, L.; Lin, C.-W.; Li, S.-T.; Wu, C.-Yi; Wong, C.-H. Efficient Convergent Synthesis of Bi-, Tri-, and Tetra-antennary
(26) Complex Type N-Glycans and Their HIV-1 Antigenicity. J. Am. Chem. Soc. 2013, 135, 15382.
(27) Shivatare, S. S.; Chang, S.-H.; Tsai, T.-I; Tseng, S. Y.; Shivatare, V. S.; Lin, Y.-S.; Cheng, Y.-Y.; Ren, C.-T.; Lee, C.-C. D.; Pawar, S.; Tsai, C.-S.; Shih, H.-W.; Zeng, Y.-F.; Liang, C.-H.; Kwong, P. D.; Burton, D. R.; Wu, C.-Y.; Wong, C.-H. Modular synthesis of N-glycans and arrays for the hetero-ligand binding analysis of HIV antibodies. Nat. Chem. 2016, 8, 338.
(28) Mong, T. K.-K.; Huang, C.-Y.; Wong, C.-H. A New Reactivity-Based One-Pot Synthesis of N-Acetyllactosamine Oligomers. J. Org. Chem. 2003, 68, 2135.
(29) Dinkelaar, J.; Duivenvoorden, B. A.; Wennekes, T.; Overkleeft, H. S.; Boot, R. G.; Aerts, J. M. F. G.; Codée, J. D. C.; van der Marel, G. A. A Preparative Synthesis of Human Chitinase Fluorogenic Substrate (4′-Deoxychitobiosyl)-4-methylumbelliferone. Eur. J. Org. Chem. 2010, 2565.
(30) Chayajarus, K.; Chambers, D. J.; Chughtai, M. J.; Fairbanks, A. J. Stereospecific Synthesis of 1,2-cis Glycosides by Vinyl-Mediated IAD. Org. lett. 2004, 6, 3797-3800.
(31) Cao, H.; Li, Y.; Lau, K.; Muthana, S.; Yu, H.; Cheng, J.; Chokhawala, H. A.; Sugiarto, G.; Zhang, L.; Chen, X. Sialidase substrate specificity studies using chemoenzymatically synthesized sialosides containing C5-modified sialic acids. Org. Biomol. Chem., 2009, 7, 5137.
(32) Lin, C.-W.; Tsai, M.-H.; Li, S.-T.; Tsai, T.-I.; Chu, K.-C.; Liu, Y.-C.; Lai, M.-Y.; Wu, C.-Y.; Tseng, Y.-C.; Shivatare, S. S.; Wang, C.-H.; Chao, P.; Wang, S.-Y.; Shih, H.-W.; Zeng, Y.-F.; You, T.-H.; Liao, J.-Y.; Tu, Y.-C.; Lin, Y.-S.; Chuang, H.-Y.; Chen, C.-L.; Tsai, C.-S.; Huang, C.-C.; Lin, N.-H.; Ma, C.; Wu, C.-Y.; Wong, C.-H. A common glycan structure on immunoglobulin G for enhancement of effector functions. Proc. Natl. Acad. Sci. U.S.A. 2015, 112, 10611.
(33) Stavenhagen, K.; Hinneburg, H.; Thaysen-Andersen, M.; Hartmann, L.; Silva, D. V.; Fuchser, J.; Kaspar, S.; Rapp, E.; Seebergera, P. H.; Kolaricha D. Quantitative mapping of glycoprotein micro-heterogeneity and macro-heterogeneity: an evaluation of mass spectrometry signal strengths using synthetic peptides and glycopeptides. J. Mass Spectrom. 2013, 48, 627.
(34) Lee, J.-C.; Greenberg, W. A; Wong, C.-H. Programmable reactivity-based one-pot oligosaccharide synthesis. Nat. Protoc. 2006, 1, 3143.

What is claimed is:

1. A monoclonal antibody comprising a saccharide containing a 3-fluoro-sialic acid, wherein the saccharide containing a 3-fluoro-sialic acid is prepared by:
conducting a glycosylation reaction by reacting a 3-hydroxy-sialic acid with a
saccharide to form an α2,6-linked 3-hydroxy-sialoside, wherein:
the 3-hydroxy-sialic acid is of formula (I): 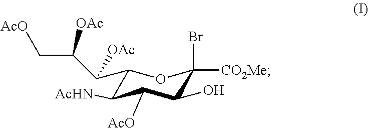
the saccharide is a compound of formula (II): 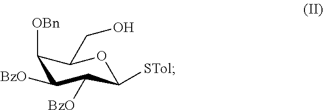
and
the α2,6-linked 3-hydroxy-sialoside is a compound of formula (III): 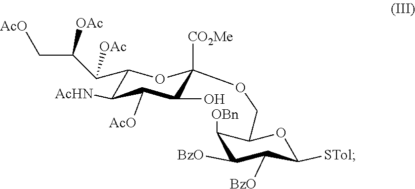
and
conducting a fluorination reaction by reacting the α2,6-linked 3-hydroxy-sialoside with a fluorinating agent to form a saccharide containing a 3-fluoro-sialic acid,
wherein the saccharide containing a 3-fluoro-sialic acid is a compound of formula (IV):

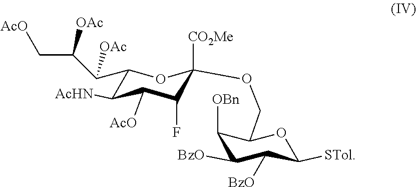

2. The monoclonal antibody of claim 1, wherein the glycosylation reaction is conducted in the presence of AgOTf and Na₂HPO₄,

3. The monoclonal antibody of claim 1, wherein the fluorinating agent is NfF,

4. The monoclonal antibody of claim 1, wherein the fluorination reaction is conducted in the presence of a catalyst.

5. The monoclonal antibody of claim 4, wherein the catalyst is DBU.

6. The monoclonal antibody of claim 1, wherein each of the glycosylation and fluorination reactions is conducted in toluene, and wherein the fluorination reaction is conducted further in the presence of TASF.

7. A method of treating a cancer, which comprises administering to a subject in need thereof an effective amount of a monoclonal antibody of claim 1.

8. A monoclonal antibody comprising a saccharide containing a 3-fluoro-sialic acid, wherein the saccharide containing a 3-fluoro-sialic acid is a compound of formula (V): 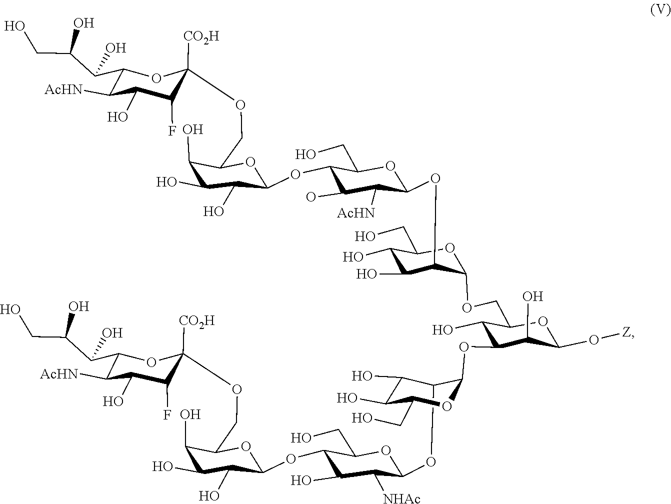
wherein Z is

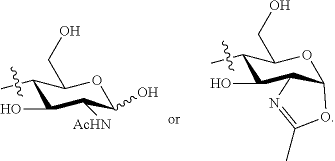

9. A method of treating a cancer, which comprises administering to a subject in need thereof an effective amount of a monoclonal antibody of claim 8.

10. A compound of formula (VI): 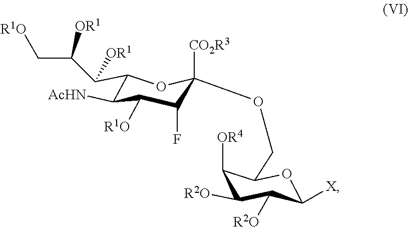
wherein R¹ is Ac or H, R² is Bz or H, R³ is methyl or H, R⁴ is Bn or H, and X is OH, a leaving group, or a saccharide.

11. The compound of claim 10, wherein the compound is of formula (IV):

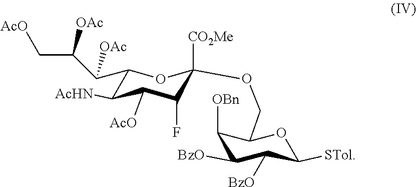

12. The compound of claim 10, wherein X is an N-glycan.

13. The compound of claim 12, wherein the compound is of formula (V): 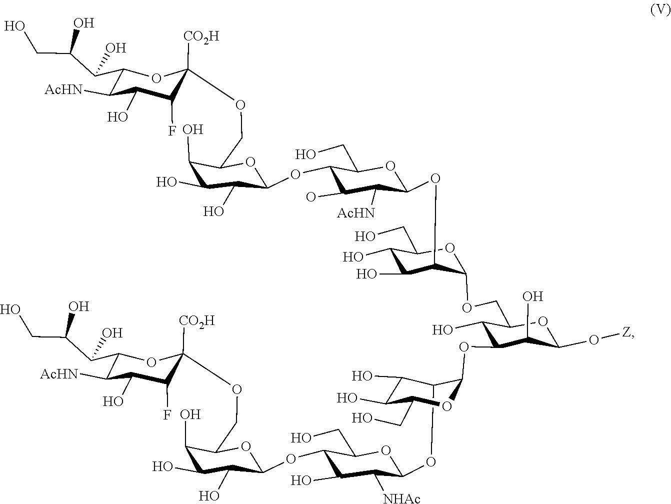
wherein Z is [chemical structure]
